(19) United States
(12) Patent Application Publication
(10) Pub. No.: US 2011/0028566 A1
(43) Pub. Date: Feb. 3, 2011

(54) COMPOSITIONS AND PRODUCTS CONTAINING CYCLOALIPHATIC DIOL ANTIMICROBIAL AGENTS AND METHODS OF USING THE COMPOSITIONS AND PRODUCTS

(75) Inventors: James Allen McCaulley, Ringoes, NJ (US); Terry Ann Oldfield, Kingsport, TN (US); Andrew Joseph Matosky, Kingsport, TN (US); Suzanne Winegar Dobbs, Kingsport, TN (US); Vicky Lynn Christian, Mount Carmel, TN (US); William Mark Barbour, Kingsport, TN (US)

Correspondence Address: POLLY C. OWEN, P.O. BOX 511, KINGSPORT, TN, 37662-5075, US

(73) Assignee: EASTMAN CHEMICAL COMPANY, Kingsport, TN (US)

(21) Appl. No.: 12/779,164

(22) Filed: May 13, 2010

Related U.S. Application Data
(60) Provisional application No. 61/178,713, filed on May 15, 2009.

(51) Int. Cl. A61K 31/047; A01N 31/04; A01N 31/06; A01P 1/00; A61K 8/34; A61P 31/00; A61P 31/04; A61P 31/10; A61Q 15/00; A61Q 5/02; A61Q 19/10; C10L 1/182; C10L 1/188; C10M 169/00; C09D 5/16; C01D 5/00

(52) U.S. Cl. 514/729; 510/119; 510/131; 44/411; 44/451; 508/532; 508/583; 523/122; 423/517

(57) ABSTRACT

Compositions comprising at least one cycloaliphatic diol antimicrobial agent and at least one other antimicrobial agent and methods of making and using these compositions are provided. The cycloaliphatic diol antimicrobial agents comprise 1,1-cyclohexanedimethanol, 1,2-cyclohexanedimethanol, 1,4-cyclohexanedimethanol, 2,2,4,4-tetramethyl-1,3-cyclobutanediol, or mixtures thereof.

### FIELD OF THE INVENTION

The invention generally pertains to antimicrobial agents, compositions and products incorporating the agents, and methods of using the compositions and products. The antimicrobial agents are 1,1-cyclohexanedimethanol, 1,2-cyclohexanedimethanol, 1,4-cyclohexanedimethanol, 2,2,4,4-tetramethyl-1,3-cyclobutanediol, and mixtures thereof.

### BACKGROUND OF THE INVENTION

Many compositions and products, including personal care, medicinal, animal care, household care, fuel, and oil, often contain water or can accumulate water from the environment. Water makes the compositions and products susceptible to microbial growth.

Antimicrobial agents are typically added to these products to limit the growth of any bacteria, yeast, or mold. Many different types of antimicrobial agents are available for this purpose. The type of antimicrobial agent and their concentration are selected based on a number of factors including the type of product being preserved, the efficacy of the antimicrobial agent, and the types of organisms that are likely to contaminate the product. If the product is likely to come into contact with humans or animals, the antimicrobial agent has to be considered for potential for causing irritation, dryness, allergy, and toxicity. Due to these and other considerations, government institutions sometimes regulate the use of antimicrobial agents.

Many glycols have been identified as having antimicrobial agent effect such that traditional antimicrobial agents can be eliminated from the products or their concentration can be reduced. Such glycols include propylene glycol, butylene glycol, pentylene glycol, 1,2-hexanediol, 1,2-octanediol, 1,5-pentanediol, methyl propanediol, and 1,3-alkanediols having 5 to 15 carbon atoms. The 1,2-hexanediol and 1,2-octanediol have been found to be particularly effective as antibacterial agents, and it has been recognized that the antibacterial activity of 1,2-alkanediols increases as the alkyl chain length increases. The hydrophobic interaction of the longer hydrocarbon chain with microorganisms is thought to contribute to their antibacterial activity. However, as the alkyl chain length increases, the water solubility of these compounds decreases. For certain products containing an immiscible organic phase (such as personal care emulsions), compounds having low water solubility are likely to migrate into the oil phase where they are less effective.

Regulations have been imposed to reduce the usage of antimicrobial agents deemed to be a threat to the environment or health safety. Therefore, it is advantageous to enhance the effectiveness of such antimicrobial agents so that less can be used in the application while maintaining the desired inhibitory effect. Cycloaliphatic diol antimicrobial agents have been found to enhance the effectiveness of antimicrobial agents and other antimicrobial agents used in various applications, including cosmetics, personal and household care, and coatings.

Thus, there is a continuing need in the art for antimicrobial agents that are effective, preferably at lower concentrations; that are safe; that cause minimal allergic reaction, irritation, and dryness at the effective concentrations; and that have a high degree of solubility in water at ambient or near ambient conditions.

### SUMMARY OF THE INVENTION

It has been surprisingly found that cycloaliphatic diol antimicrobial agents have been found to enhance the effectiveness of antimicrobial agents used in various applications, including, but not limited to cosmetics, personal care, household care, and other coatings. The use of cyloaliphatic diol antimicrobial agents alone is described in U.S. patent application Ser. No. 12/341,462, entitled Antimicrobial Agents, Compositions and Products Containing the Same, and Methods of Using The Compositions and Products, herein incorporated by reference to the extent it does not contradict the disclosure herein.

In a first aspect, the invention provides a method for enhancing the effectiveness of a least one antimicrobial agent in reducing or inhibiting microbial growing in an aqueous composition. The method comprises adding a cycloaliphatic antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol to the composition and at least one other antimicrobial agent to the aqueous composition.

In a second aspect, the invention provides a composition comprising (a) a fuel or oil selected from diesel, biodiesel, a mixture of diesel and biodiesel, aviation fuel, hydraulic oil, lubrication oil, vegetable oil, crude oil, transmission fluid, heating oil, or kerosene; and (b) at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and c) at least one other antimicrobial agent.

In a third aspect, the invention provides a personal care product comprising at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent.

In a fourth aspect, the invention provides a medicated product comprising a medicinal substance; at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and at least one other antimicrobial agent.

In a fifth aspect, the invention provides an animal care product comprising at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent.

In a sixth aspect, the invention provides a household care product comprising at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent.

In a seventh aspect, the invention provides a method for providing residual antimicrobial activity to a surface. The method comprises topically applying the personal care, medicated, animal care, or household care product mentioned above to the surface, and optionally removing any excess amounts of the product from the surface.

In an eighth aspect, the invention provides a method for preventing or reducing odor from the presence of bacteria or fungi on a mammalian surface. The method comprises topically applying the personal care, medicated, or animal care product mentioned above to the mammalian surface, and optionally removing any excess amounts of the product from the mammalian surface.

In a ninth aspect, the invention provides a method for providing antimicrobial activity to a film, fiber, molded or extruded article, or composite material made of fibers, polymers, adhesives, and/or gypsum. The method comprises incorporating an antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent into the film, fiber, molded or extruded article, or composite material during its manufacturing process.

### DETAILED DESCRIPTION OF THE INVENTION

According to a first aspect, the invention provides a method for enhancing the effectiveness of at least one antimicrobial agent in reducing or inhibiting microbial growth in an aqueous composition. The method comprises adding at least one cycloaliphatic diol antimicrobial agent selected from the group consisting of 1,1-cyclohexanedimethanol (1,1-CHDM), 1,2-cyclohexanedimethanol (1,2-CHDM), 1,4-cyclohexanedimethanol (1,4-CHDM), and 2,2,4,4-tetramethyl-1,3-cyclobutanediol (TMCBD) and at least one second antimicrobial agent to the aqueous composition.

The aqueous composition can be any composition that contains water and that is susceptible to microbial growth. Examples of such compositions include fuel or oil compositions, personal care products, medicated products, animal care products, and household care products. Thus, in addition to water, the aqueous composition can contain, for example, an organic compound such as hydrocarbons, triglycerides, fatty acids, fatty acid alkyl esters, fatty alcohols, polyglycol ethers, alkyl glycol ethers, alkyl glycol esters, alkyl glycol ether esters, alkyl amines, alkyl amides, and mixtures thereof. Other examples of the organic compound include diesel, biodiesel, a mixture of diesel and biodiesel, aviation fuel, hydraulic oil, lubrication oil, vegetable oil, crude oil, transmission fluid, heating oil, or kerosene.

In one embodiment, the organic compound and the water in the aqueous composition are miscible. In another embodiment, the organic compound and the water in the aqueous composition are in separate liquid phases. In this latter case, the antimicrobial agent preferably reduces or inhibits microbial growth at the interface between the organic phase and the aqueous phase in the aqueous composition.

The amount of the cycloaliphatic diol antimicrobial agents and the other antimicrobial agent present in the aqueous composition can vary depending on various factors including the application of the aqueous composition and the degree of microbial protection desired. Typically, the amount of the cycloaliphatic diol antimicrobial agent present in the coating composition will be in the range of about 0.1 to about 5 weight percent, based on the weight of the aqueous composition. Preferably, the cycloaliphatic diol antimicrobial agent is present in the range of about 0.3 to about 4 weight percent, based on the weight of the aqueous composition. Other ranges are from about 0.1 to about 3, about 0.5 to about 4, and about 1 to about 3.5, based on the weight of the aqueous composition.

Any second antimicrobial agent known in the art can be utilized in this invention. Table 1 gives examples of specific antimicrobial agents used in applications such as cosmetics/personal care and coatings, and the class of antimicrobials each represents.

TABLE 1
Selected Antimicrobial Agents Representing Various Classes of
Antimicrobials

| | Antimicrobial Agent | Represents Class . . . | Others in Same Class |
|---|---|---|---|
| 1 | Phenoxyethanol (PE) | Phenolics (excl | benzyl alcohol, phenethyl alcohol |
| | | parabens) | |
| 2 | Caprylyl Glycol (CG) | 1,2-Alkanediols | 1,2-pentanediol, 1,2-hexanediol, |
| | (1,2-octanediol) | | 1,2-decanediol, 3-[(2- |
| | | | ethylhexyl)oxy]-1,2-propanediol |
| | | | (ethylhexylglycerin) |
| 3 | Methylparaben (MP) | Parabens | Ethyl, propyl, butyl, isopropyl, |
| | | | isobutyl, & benzyl paraben and |
| | | | their sodium salts |
| 4 | Methylisothiazolinone | Isothiazolinones | Methylchloroisothiazolinone (MCIT) |
| | (MIT) | | |
| 5 | 9:1 wt ratio Benzyl | ECOCERT approved | Benzoic acid & its esters & salts; |
| | Alcohol (BA) and | antimicrobial agents | salicylic acid & its salts; sorbic acid; |
| | Dehydroacetic Acid | (and blends with | dehydroacetic acid & its salts |
| | (DHA) | organic acids) | |
| 6 | Chlorphenesin (CP) | Halogenated | chloroxylenol; triclosan; |
| | | aromatic compounds | dichlorobenzyl alcohol; climbazole; |
| | | | triclocarban |
| 7 | DMDM Hydantoin | Formaldehyde | Imidazolidinyl urea; diazolidinyl |
| | (DMDMH) | releasers | urea; quaternium-15; methenamine |
| 8 | Iodopropynyl | Halogenated non- | 2-bromo-2-nitropropane-1,3-diol; |
| | butylcarbamate | aromatic compounds | chloroacetamide; chlorobutanol; |
| | (IPBC) | | methyldibromo glutaronitrile |
| 9 | Benzisothiazolinone | Isothiazolinones | MIT, MCIT (see above) |
| | (BIT) | | |
| 10 | 1:1 MIT:BIT for | Isothiazolinones | (see above) |
| | coatings | | |
| 11 | Benzalkonium | Quaternia | benzethonium chloride, |
| | Chloride | | chlorhexidine, polyaminopropyl |
| | | | biguanide |

The amount of the second antimicrobial agent can vary depending on various factors including the application of the aqueous composition and the degree of microbial protection desired. In one embodiment of the invention, the amount of the second antimicrobial agent can vary as shown in Table 2 below.

TABLE 2

| Antimicrobial Agent | in class of | Concentration range, wt % | | |
|---|---|---|---|---|
| phenoxyethanol | Phenolics | 0.05-1.0 | 0.1-0.6 | 0.2-0.5 |
| benzyl alcohol | Phenolics | 0.05-1.0 | 0.1-0.6 | 0.2-0.5 |
| caprylyl glycol | 1,2-alkanediols | 0.03-0.8 | 0.05-0.6 | 0.1-0.4 |
| | (C3-C10) | | | |
| methyl paraben | parabens | 0.02-0.4 | 0.03-0.20 | 0.04-0.10 |
| methylisothiazolinone | Isothiazolinones | 0.0005-0.020 | 0.0010-0.010 | 0.0015-0.005 |
| benzisothiazolinone | Isothiazolinones | 0.0005-0.20 | 0.0010-0.10 | 0.0015-0.05 |
| dehydroacetic acid | organic acids | 0.005-0.5 | 0.01-0.4 | 0.02-0.20 |
| chlorphenesin | halogenated | 0.01-0.4 | 0.02-0.30 | 0.05-0.20 |
| | aromatic | | | |
| | compounds | | | |
| DMDM Hydantoin | formaldehyde | 0.02-0.6 | 0.05-0.4 | 0.10-0.30 |
| | releasers | | | |
| IPBC | halogenated | 0.0001-5.0 | 0.001-1.5 | 0.02-0.5 |
| | aliphatic | | | |
| | compounds | | | |

The manner in which the cycloaliphatic diol antimicrobial agent and the other antimicrobial agent is added to the aqueous composition is not particularly limiting. For example, the cycloaliphatic diol antimicrobial agent and other antimicrobial agent may be added to the aqueous composition by simply combining the agents with the aqueous composition and mixing the ingredients. Alternatively, the cycloaliphatic diol antimicrobial agent, due to its high solubilizing power, may be used as a solvent for one or more of the ingredients of the aqueous composition before it is mixed with the remainder of the composition ingredients.

In another embodiment, the cycloaliphatic diol antimicrobial agent may be added to the aqueous composition by first mixing the cycloaliphatic diol agent with a solvent that is immiscible with water and then combining the agent-solvent mixture with the aqueous composition.

The cycloaliphatic diol antimicrobial agent itself may be a soft solid at room temperature. Therefore, to facilitate mixing and/or handling, the cycloaliphatic diol agent may first be diluted with up to 10 wt % or more of water before it is combined with the aqueous composition or the ingredients thereof.

The method of the invention enhances the effectiveness of the antimicrobial agent to reduce or inhibit microbial growth of various kinds including biofilms.

According to a second aspect, the invention provides a composition comprising (a) a fuel or oil selected from diesel, biodiesel, a mixture of diesel and biodiesel, aviation fuel, hydraulic oil, lubrication oil, vegetable oil, crude oil, transmission fluid, heating oil, or kerosene; and (b) an antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and (c) at least one other antimicrobial agent.

The amount of the cycloaliphatic diol antimicrobial agent and the other antimicrobial agent present in the fuel or oil composition can vary depending on various factors including the degree of microbial protection desired. Generally, the cycloaliphatic diol antimicrobial agent can be present in an amount of about 0.01 to 1 weight percent, based on the total weight of the fuel or oil composition. The cycloaliphatic diol antimicrobial agent can also be present in an amount of about 0.02 to 0.5 weight percent, based on the total weight of the fuel or oil composition or even in an amount of about 0.05 to 0.2 weight percent based on the total weight of the fuel or oil composition. The concentration range for the cycloaliphatic diol antimicrobial agent in the fuel or oil can also be determined by those skilled in the art by determining the partition coefficient of the cycloaliphatic diol antimicrobial agent for the fuel or oil and water mixture, and then calculating the amount to add to the fuel or oil to achieve 1 to 5% by weight of the antimicrobial agent in the water that may contaminate the oil or fuel.

The fuel or oil composition may contain typical additives such as detergents, octane boosters, oxygenates, corrosion inhibitors, lubricants, metal deactivators, antioxidants, antiknock agents, dyes, combustion catalysts, burn rate modifiers, deposit control additives, friction modifiers, viscosity modifiers, antiwear additives, pour point depressants, anti-foam agents, seal conditioners, extreme pressure agents, dispersants, and wax crystal modifiers.

According to a third aspect, the invention provides a personal care product comprising at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent. The cycloaliphatic diol antimicrobial agent can also be present in an amount of about 1 to 3 weight percent, based on the total weight of the personal care product.

In one embodiment, the personal care product contains water and the weight percentage of the antimicrobial agent is based on the amount of water in the product.

In another embodiment, the personal care product is anhydrous and the weight percentage of the antimicrobial agent is based on the total weight of the product.

Examples of personal care products according to the invention include hand soaps, hand sanitizers, body washes, shower gels, shampoos, conditioners, face creams, body lotions, underarm deodorants, mouthwash, toothpaste, cosmetics, contact lens solutions, hairstyling products, acne treatment products, fragrances, and foot, sock, or shoe deodorizing compositions.

According to a fourth aspect, the invention provides a medicated product comprising a medicinal substance, at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol, and at least one other antibacterial agent. The cycloaliphatic diol antibacterial agent can also be present in an amount of about 1 to about 3 weight percent, based on the total weight of the medicated product.

In one embodiment, the medicated product contains water and the weight percentage of the antimicrobial agent is based on the amount of water in the product.

In another embodiment, the medicated product is anhydrous and the weight percentage of the antimicrobial agent is based on the total weight of the product.

Examples of medicated products according to the invention include acne treatment products, wound care products, and transdermal patches.

Examples of medicinal substances that can be included in the medicated product of the invention include skin rejuvenating products such as salicylic acid, glycolic acid, Vitamin A, Vitamin E, hyaluronic acid, caffeine, aloe vera, Co-enzyme Q10, collagen, and derivatives thereof; anesthetics such as benzocaine or lidocaine; antifungal products such as ketoconazole or fluconozole and the like; anti-inflammatory or anti-itch substances such as hydrocortisone, benadryl and the like, pain medications such as morphine sulfate; and the like, antibiotics, such as amoxicillin, penicillin, trimethoprim, bactrim, sulfamethizole, erythromycin, polymyxin B Sulfate and the like; hormones such as estradiol, progestin, progesterone, testosterone and the like; anti-anxiety medications; anti-depressants or anti-Parkinson's medication, such as selegeline and the like; anti-spasmotic medications such as oxybutynin; anti-convulsive medications such as carbamazepine, anti-motion sickness medication such as scopoloamine; anti-smoking medications such as nicotine; anti-cancer medications such tamoxiphen or 5-fluorouracil, anti-dandruff medications, anti-perspirant medications and actives, and anti-viral medications such as vaccine ingredients.

According to a fifth aspect, the invention provides an animal care product comprising a cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent. The cycloaliphatic diol antimicrobial agent can also be present in an amount of about 1 to 3 weight percent, based on the total weight of the animal care product.

In one embodiment, the personal care product contains water and the weight percentage of the antimicrobial agent is based on the amount of water in the product.

In another embodiment, the animal care product is anhydrous and the weight percentage of the antimicrobial agent is based on the total weight of the product.

Examples of animal care products according to the invention include shampoos, conditioners, and fragrances.

According to a sixth aspect, the invention provides a household care product comprising at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent. The cycloaliphatic diol antimicrobial agent can also be present in an amount of about 1 to 3 weight percent, based on the total weight of the household care product.

In one embodiment, the household care product contains water and the weight percentage of the antimicrobial agent is based on the amount of water in the product.

In another embodiment, the household care product is anhydrous and the weight percentage of the antimicrobial agent is based on the total weight of the product.

Examples of household care products according to the invention include surface cleaners, air or surface deodorizers, laundry care products, dishwashing detergents, and rinse aids.

According to a seventh aspect, the invention provides a method for providing residual antimicrobial activity to a surface. The method comprises topically applying the personal care, medicated, animal care, or household care product of the invention to the surface, and optionally removing any excess amounts of the product from the surface.

The treated surface may be the skin or hair of a human or animal, or inanimate objects such as door handles, floors, counter tops, desktops, and furniture.

These steps may be repeated as often as desired, such as 2 to 6 times daily.

In one embodiment, the surface has a biofilm on it before the product is applied.

According to an eighth aspect, the invention provides a method for preventing or reducing odor from the presence of bacteria or fungi on a mammalian surface. The method comprises topically applying the personal care, medicated, or animal care product of the invention to the mammalian surface, and optionally removing any excess amounts of the product from the mammalian surface.

The mammalian surface can be anywhere on the exposed surface of a mammal including hands, feet, underarm, groin, and teeth.

These steps may be repeated as often as desired, such as 2 to 6 times daily.

According to a ninth aspect, the invention provides a method for providing antimicrobial activity to a film, fiber, molded or extruded article, or composite material made of fibers, polymers, adhesives, and/or gypsum. The method comprises incorporating an cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent into the film, fiber, molded or extruded article, or composite material during its manufacturing process.

The cycloaliphatic diol antimicrobial agent and/or the other antimicrobial agent could be dissolved in a plasticizer, such as diethylphthalate (DEP) and mixed directly into the powdered plastic material to be extruded or thermoformed during application. Alternatively, the cycloaliphatic diol antimicrobial agent and/or the other antimicrobial agent could be dissolved in a common solvent or co-solvent along with the polymer, such as cellulose acetate and cast as a thin film to dry. The powder can then be cryogenically ground to form particles of the correct dimensions.

The amount of the cycloaliphatic diol antimicrobial agent and the other antimicrobial agent present in the film, fiber, molded or extruded article, or composite material can vary depending on various factors including the degree of microbial protection desired. Generally, the cycloaliphatic diol antimicrobial agent can be present in an amount of about 1 to about 5 weight percent, based on the total weight of the composition. The cycloaliphatic diol antimicrobial agent can also be present in an amount of about 1 to about 3 weight percent, based on the total weight of the composition.

In another embodiment, the method of the invention is effective to prevent a biofilm from forming on a surface of the film, fiber, molded or extruded article, or composite material.

This invention can be further illustrated by the following examples of preferred embodiments thereof, although it will be understood that these examples are included merely for purposes of illustration and are not intended to limit the scope of the invention. In the following examples, all percentages are by weight unless otherwise indicated. Additionally, CHDM-D denotes anhydrous 1,4-cyclohexanedimethanol, and CHDM-D90 denotes a mixture of 90 wt % 1,4-CHDM and 10 wt % water.

The invention has been described in detail with particular reference to preferred embodiments thereof, but it will be understood that variations and modifications can be effected within the spirit and scope of the invention.

### EXAMPLES

Below is a summary of the results of the examples. In these experiments, 1,4-CHDM was tested alone and in combination with the metal chelator EDTA (ethylenediaminetetraacetic acid disodium salt) and with commonly-used biocides, PE and CG. Also, 1,4-CHDM and its structural isomers, TMCD (2,2,4,4-tetramethyl-1,3-cyclobutanediol) and 1,3-CHDM were tested alone and each in combination with BIT. 1,1-CHDM was also tested and showed improved efficacy over 1,4-CHDM.

Experiments with 1,4-CHDM in combination with EDTA, PE, and CG showed synergistic antimicrobial effects (see Table 5). The specific findings were as follows:
0.2 wt % EDTA in combination with 1,4-CHDM provided a synergistic effect against most organisms, with the effect being most apparent at 1.25 wt % 1,4-CHDM.
Complete kills were achieved against P. aeruginosa for 1,4-CHDM with both PE and CG.
Synergistic effects are seen against E. coli for 1,4-CHDM at 1.25 wt % and 2.5 wt % with PE and at 1.25 wt % with CG.
1.25 wt % 1,4-CHDM in combination with phenoxyethanol (PE) and with caprylyl glycol (CG) showed a synergistic effect against Staphylococcus aureus and Staphylococcus epidermidis at 3 days incubation; thus providing a quicker response compared to PE and CG alone. This quick response was also seen against Streptococcus mutans and Burkholderia cepacia with PE and against B. subtilis with CG.
2.5 wt % 1,4-CHDM in combination with PE and with CG showed a synergistic effect against Aeromonas sp.
1.25 wt % and 2.5 wt % 1,4-CHDM with 0.25% PE showed a synergistic response against Microsporum canis.

Experiments with 1,4-CHDM, TMCD, and 1,3-CHDM, each in combination with BIT also showed synergistic antimicrobial effects (see Table 6). The specific findings are as follows:
In general, the combinations of each cycloaliphatic diol antimicrobial agent with BIT showed synergies against most organisms.
1,4-CHDM at 0.5 wt % to 2.5 wt % showed synergism with 0.05 wt % and 0.2 wt % BIT against fungi and Gram negative bacteria.
Surprisingly, while 1,4-CHDM with BIT showed synergism against C. albicans, TMCD and 1,3-CHDM did not.
TMCD at 0.5% to 2.5% with 0.05% BIT showed synergism against S. aureus and S. epidermidis, whereas 1,4- and 1,3-CHDM did not.
Synergism against S. mutans was not very apparent because BIT alone was highly effective.
1,4-CHDM and TMCD with BIT showed synergism against B. subtilis, whereas 1,3-CHDM did not.

Given the results for EDTA, the concentration range for disodium EDTA that can show synergism with 1,4-CHDM can be about 0.1 to 0.3% based on the total formulation.

Broth culture synergy experiments with 1,4-CHDM in combination with nine common antimicrobial agents (1-9 in Table 1) showed unexpected synergistic activity for a range of microorganisms. The preferred concentration range for 1,4-CHDM is 0.1 to 5 weight percent, more preferably 0.3 to 3.3 weight percent.

The unexpected results are summarized as follows:
Broad synergism, for bacteria, yeast and mold, was shown for phenoxyethanol in combination with 1,4-CHDM. The preferred composition comprises phenoxyethanol and 1,4-CHDM at a weight ratio from 1:1 to 1:100, more preferably from 1:3 to 1:33.
Broad synergism, for bacteria, yeast and mold, was shown for benzyl alcohol:dehydroacetic acid in combination with 1,4-CHDM. The preferred composition comprises benzyl alcohol:dehydroacetic acid and 1,4-CHDM at a weight ratio from 1:1 to 1:500, more preferably from 1:4 to 1:50.
IPBC in combination with 1,4-CHDM showed strong synergism for P. aeruginosa. The preferred composition comprises IPBC and 1,4-CHDM at a weight ratio from 1:2 to 1:1000, more preferably from 1:7 to 1:220.
MIT in combination with 1,4-CHDM showed strong synergism for P. aeruginosa. The preferred composition comprises MIT and 1,4-CHDM at a weight ratio from 1:100 to 1:10,000, more preferably from 1:1587 to 1:3333.
BIT in combination with 1,4-CHDM showed strong synergism for P. aeruginosa. The preferred composition comprises BIT and 1,4-CHDM at a weight ratio from 1:5 to 1:1000, more preferably from 1:27 to 1:250.
A MIT:BIT mixture in combination with 1,4-CHDM showed strong synergism for P. aeruginosa. The preferred composition comprises BIT:MIT and 1,4-CHDM at a weight ratio from 1:100 to 1:10,000, more preferably from 1:613 to 1:1961.
Caprylyl glycol in combination with 1,4-CHDM was synergistic for both fungi tested, C. albicans and A. niger. The preferred composition comprises caprylyl glycol and 1,4-CHDM at a weight ratio from 1:1 to 1:500, more preferably from 1:20 to 1:50.
Chlorphenesin in combination with 1,4-CHDM was synergistic for C. albicans. The preferred composition comprises chlorphenesin and 1,4-CHDM at a weight ratio from 1:1 to 1:100, more preferably from 1:10 to 1:16.
DMDM hydantoin in combination with 1,4-CHDM was synergistic for C. albicans. The preferred composition comprises DMDM hydantoin and 1,4-CHDM at a weight ratio from 1:1 to 1:500, more preferably from 1:7 to 1:50.

### Example 1

### Antimicrobial Activity of CHDM in Combination with Other Antimicrobial Agents

### Procedure: Microbial Challenge Testing in BPW

The microorganisms used in challenge tests are given in Table 3, designated as either ATCC (American Type Culture Collection) or wild type. These wild type organisms were problematic organisms previously isolated from chemical products. In the description for each organism, the bacteria are indicated as either GN (Gram negative) or GP (Gram positive).

TABLE 3
Challenge Organisms for Tests in BPW

| | | ATCC or |
|---|---|---|
| | | Wild |
| Organism | Description | Type? |
| Aspergillus niger | Black mold; common on fruits, | Wild type |
| | vegetables, and external surfaces; | |
| | allergenic; opportunistic pulmonary | |
| | infections | |
| Candida albicans | Yeast; opportunistic oral and genital | Wild type |
| | infections | |
| Pseudomonas | GN; opportunistic human pathogen | Wild type |
| aeruginosa | (especially cystic fibrosis and burn | |
| | patients) | |
| Escherichia coli | GN; found in intestines; can cause | ATCC |
| | gastroenteritis | #25922 |
| Staphylococcus | GP; common cause of skin infections; | ATCC |
| aureus | food poisoning | #25923 |
| Staphylococcus | GP; usually non-pathogenic; cause of skin | ATCC |
| epidermidis | odor | #12228 |
| Streptococcus | GP; found in mouth; contributes to tooth | ATCC |
| mutans | decay | #35668 |
| Bacillus subtilis | GP; non-pathogenic; spores; causes | Wild type |
| | spoilage | |
| Burkholderia | GN; can cause pneumonia in immuno- | Wild type |
| cepacia | compromised individuals | |
| Proteus vulgaris | GN; opportunistic pathogen, known to | Wild type |
| | cause urinary tract infections | |
| Aeromonas sp. | GN; some species are pathogenic, | Wild type |
| | causing wound infections | |

All of the microorganisms were grown in Tryptose Soy Broth (TSB), DIFCO™ available from Becton, Dickinson and Company, containing 1% dextrose. Aspergillus niger and Candida albicans were incubated at 22° C.±2° C. for at least 96 hours. All bacteria were incubated at 35° C.±2° C. in a humidified incubator for at least 96 hours.

Aspergillus niger and Candida albicans were also grown on Sabouraud dextrose agar (SDA) at 22° C.±2° C. for 7 to 14 days or until full sporulation was achieved.

### Determining the Amount of Challenge Inoculum

The following procedure was followed to determine the amount of each challenge material (inoculum broth) required to produce a 10⁸ cfu/mL challenge, which is equivalent to a final test-sample microbial concentration of 10⁵ to 10⁶ cfu (colony-forming units)/mL.

Using a sterile serological pipette, 1 mL of the growth from each TSB culture was transferred into tubes containing 9 mL phosphate buffer (pH 7.2) and mixed thoroughly. This process was repeated to make serial 1:10 dilutions. Then 0.1 mL of each sample and dilution was inoculated onto agar plates to produce the equivalent of a further 1:10 dilution. C. albicans and A. niger were inoculated onto SDA and bacteria were inoculated onto Plate Count Agar (PCA), DIFCO™ available from Becton, Dickinson and Company. The 0.1 mL aliquots were evenly distributed on the plates using the spread-plate technique. The spread-plate technique is performed by spreading the aliquot over the entire plate surface using a sterile spreading rod while rotating the plate with a rotary auto-plater. After the inoculum was absorbed completely, each plate was inverted and incubated (fungi at 22° C.±2° C. and bacteria at 35° C.±2° C.).

After incubation for at least 48 hours, colonies that had developed on the agar plates were counted and recorded with the corresponding dilution. If counting had to be delayed temporarily, plates were refrigerated, preferably no more than 24 hours, until they could be counted. The number of cfu/mL was determined by multiplying the count by the dilution factor for that particular plate.

Turbidity in Nepholemetric Turbidity Units (NTU) was measured for each serial dilution using the HF-Micro 100 Model Turbidimeter. For each microorganism, the plate counts were compared to the turbidity readings. For Candida albicans and all bacteria, the 1:10 dilution having a turbidity reading of 34 to 38 NTU equated to a final test-sample concentration of 10⁵ to 10⁶ cfu/mL. For Aspergillus niger, the 1:10 dilution having a turbidity reading of 25 to 29 NTU achieved the final test-sample concentration of 10⁵ to 10⁶ cfu/mL.

Harvesting Aspergillus niger Cultures and Dislodging Spores

Aspergillus niger cultures were harvested and spores dislodged from the SDA on which they were grown by rubbing the growth gently with a sterile inoculating loop. The spores were then mixed into the broth culture that had been incubated with a sterile magnetic stir bar to reduce pellicle formation. The spore-culture mixture was filtered repeatedly through sterile, non-absorbent cotton and harvested repeatedly, adjusting vegetative cells and spores to a level of 1.0×10⁸. A hemocytometer was used to verify the final challenge concentration.

Harvesting Candida albicans Cultures

On the day of challenge, the Candida albicans inoculum broth was poured through non-absorbent sterile gauze and centrifuged. The pellicle was then diluted with phosphate buffer (pH 7.2) until the desired turbidity was reached. Using a hemocytometer, a determination was made whether the challenge contained the desired concentration. Dilutions were made through 1.0×10⁸ and three SDA spread plates were inoculated with 0.1 mL of each dilution. The plates were incubated for at least 48 hours and challenge counts were confirmed (i.e., 10⁵ to 10⁶ cfu/mL).

### Preparation of Test Substrates

Control substrates (“broth alone”) were prepared for each microorganism separately in triplicate by adding 13.5 mL of BPW (pH 7.0) containing 1% (w/v) dextrose to each 20-mL glass tube; then adding 1.5 mL challenge material to produce a final concentration at time zero of 10⁵ to 10⁶ cfu/mL and a total volume of 15 mL.

Test sample substrates were prepared containing each test material (1,4-CHDM, etc) or combination of test materials at the concentrations shown in Tables 5 and 6. Sample substrates were prepared in triplicate, except those substrates containing PE or CG which were prepared in duplicate. Substrates were prepared by adding BPW containing 1% (w/v) dextrose to each 20-mL glass tube, then adding the test material in the amount appropriate to achieve the desired weight/volume percent (g/100 mL) and to obtain a total volume of BPW with dextrose and test material of 13.5 mL. Then 1.5 mL challenge material was added to produce a final concentration at time zero of 10⁵ to 10⁶ cfu/mL of the respective organism and a total test sample substrate volume of 15 mL.

### Incubation and Subcultures

After mixing, all challenged substrates were incubated at 35° C.±2° C. for 14 days and then at ambient room temperature.

Subcultures were performed at 3, 13 or 14, and 30 days as follows:

A 0.1-mL aliquot was removed from each challenged substrate. The turbidity of the sample was determined and, if needed, the sample was diluted with phosphate buffer (pH 7.2) to produce readable plate counts (see “Plate Counts” below). Aspergillus niger and Candida albicans were subcultured onto SDA and grown at 22° C.±2° C. The bacteria were subcultured onto PCA and incubated at 35° C.±2° C. in a humidified incubator. Negative results were not reported before 96-hours incubation and counts were performed after a minimum of 48-hours incubation.

The identity of the microorganisms was confirmed by Gram stain (for bacteria) or lactophenol cotton blue stain (for fungi) whenever contamination was suspected. INT dye (i.e., 2-(4-iodophenyl)-3-(4-nitrophenyl)-5-phenyl tetrazolium chloride) reduction, Gram staining, and the ATP (Adenosine Triphosphate) analyses were used whenever negative results were questionable (e.g., cloudiness in the tube).

### Plate Counts

For diluted samples, plates producing 22 to 220 colonies per plate were counted and the count was multiplied by the dilution factor. Based on the plate count and dilution factor a grade code was assigned. Because colonies of Aspergillus niger clump together, accurate counts could not be achieved easily by dilution.

### Grade Code Assignment

For A. niger (based on 0.1 mL aliquot plated volume):

| Grade | Definition |
|---|---|
| 0 | No growth detected (<1 colony) |
| 1 | Countable (1 to 10 colonies) |
| 2 | Countable (11 to 100 colonies) |
| 3 | Individual colonies not countable; >75% of plate covered with |
| | growth |
| 4 | Plate not countable; continuous mat of growth |
| 5 | Obvious extreme growth (even macroscopically) in tube |

For C. albicans and all bacteria (based on 0.1 mL plated volume):

| Grade | Definition |
|---|---|
| 0 | No growth detected or <1 colony (thus <10 cfu/mL) |
| 1 | 1 to 51 colonies counted (thus 10 to 510 cfu/mL) |
| 2 | 52 to 100 colonies counted (thus 520 to 1000 cfu/mL) |
| 3 | 101 to 1000 colonies (thus 1000 to 10,000 cfu/mL) |
| 4 | 1001 to 10,000 colonies (thus 10,000 cfu/mL to 100,000 |
| | cfu/mL) |
| 5 | More than 10,000 colonies estimated (thus >100,000 cfu/mL) |

Procedure: Challenge Testing with Pathogenic Fungi in SDB

The pathogenic fungi used in challenge tests are given in Table 4. Both M. canis and Trichophyton rubrum were grown on Sabouraud dextrose broth (SDB) (pH 5.6), while Malassezia fufur was grown in SDB supplemented with 2% (v/v) of olive oil and 0.2% (v/v) of Tween™ 80; incubation was at 22° C.±2° C. under continuous agitation by stirring for 10 days. The organisms were grown to a cell concentration of between 10³ and 10⁴ cfu/mL. The actual inoculation cell concentration of these challenges was determined by diluting in sterile buffer water and (spread-plate method) plating for enumeration. The results of these counts for the challenge organisms are given in Table 4.

TABLE 4
Challenge Organisms and Inoculation Cell Concentration

| | | Inoculation Cell |
|---|---|---|
| Organism | Causes . . . | Concentration |
| Microsporum canis (ATTC | Ring-worm in cats, | 46,000 cfu/g |
| #9084) | dogs, and occasionally | |
| | in humans | |
| Trichophyton rubrum | Athlete's foot, jock itch | 1,300 cfu/g |
| (ATCC #10218) | | |
| Malassezia furfur (ATCC | Dandruff | ND* |
| #96809) | | |

*Note: The M. furfur culture was very turbid and viable, but plating onto SDA (with olive oil and Tween ™ 80) for enumeration did not give countable colonies.

Challenge organisms were used to inoculate tubes containing each test material (CHDM, etc) or combination of test materials, at concentrations given in Table 5, in SDB (or for M. furfur, in SDB supplemented with olive oil and Tween™ 80). The inoculations were in the amount of 1.5 mL aliquots of each culture with static incubation at 22±2° C. Subcultures were made after 3-, 14-, and 30-days incubation. All challenges were conducted in triplicate. In the case of M. canis, the growth response was assessed by the visual presence or absence of growth in the tubes; in the case of T. rubrum, a respiratory dye (0.2% w/v aqueous INT solution: 2-(4-iodophenyl)-3-(4-nitrophenyl)-5-phenyl tetrazolium chloride) was added to the tubes, turning red if the organism was viable; and, finally, in the case of M. furfur, the growth response was assessed based upon visible pellicle formation in the tubes at the meniscus. The fungal growth in each tube was assigned a grade code as follows:

### Grade Code Definitions

0: No visible growth
1: Some growth in tube
2: Moderate growth in tube
3: Good growth in tube
4: Extreme growth in tube

### Results

The experimental results for challenge testing of CHDM with EDTA, PE, and CG are given in Table 5. Results for 1,4-CHDM, TMCD, and 1,3-CHDM with BIT are given in Table 6. All test material concentrations are given as w/v %. The grade codes given are the average of the grade codes for duplicate or triplicate samples. In most cases, replicates had the same grade code value. Note that Table 5 gives results for microbial challenges in both BPW and SDB (pathogenic fungi); whereas Table 6 gives results for only BPW, because BIT was not tested with pathogenic fungi. Also note in Tables 5 and 6 that many data rows are repeated to make it easier to compare the results for combinations of components to the results for the individual components.

Table 5 compares the antimicrobial activity of 1,4-CHDM (AB) alone to 1,4-CHDM in combination with EDTA, phenoxyethanol (PE), and caprylyl glycol (CG). CHDM at 0.5% was tested with EDTA, and at 1.25 and 2.5% with EDTA, PE, and CG. (Note that the combination of CHDM with only EDTA was not tested against pathogenic fungi.) Results are also given for EDTA, PE, and CG alone, so the result for each mixture can be compared to the antimicrobial activity for each of its components individually. This comparison provides an indication of combinations that may provide a synergistic effect. Possible synergies were determined by treating the grade code as an estimate of the log of the microbial count (log of cfu/mL). Log reductions can then be estimated by subtracting each grade code from the grade code for broth alone (grade code 5, except for the pathogenic fungi). If the log reduction for a combination of components is greater than the log reductions for the individual components added together, then that combination showed a synergistic effect. The result is considered synergistic in light of the components alone for that organism and days incubation.

Table 6 compares the antimicrobial activity of the glycols: 1,4-CHDM, TMCD, and 1,3-CHDM alone and each in combination with 1,2-benzisothiazolin-3-one (BIT). The glycols were tested at 0.5, 1.25, and 2.5%, each with 0.05 and 0.2% BIT. Results are also given for BIT alone, so the result for each mixture can be compared to the antimicrobial activity for each of its components individually. This comparison provides an indication of combinations that may show synergy. Synergies were determined in the same way as described above for Table 5. Note that neither BIT alone nor combinations with BIT were tested against pathogenic fungi.

The results of this study indicate that the antimicrobial activity of 1,4-CHDM can be enhanced by using it in combination with small amounts (0.2%) of the metal chelator, EDTA (disodium salt). Conversely, 1,4-CHDM can be used with the common cosmetic biocides, phenoxyethanol and caprylyl glycol, to enhance their antimicrobial activity against the more common or problematic microorganisms.

1,2-Benzisothiazolin-3-one (BIT) can be moderately irritating to the skin and can be a skin sensitizer, and therefore, is used to a very limited extent in cosmetics. However, it is used in household cleaning and laundry products. The results of this study indicate that both 1,4-CHDM and TMCD can provide significant enhancement to the antimicrobial activity of BIT.

As mentioned previously, synergies (as indicated in Tables 5 and 6) were determined by treating the grade codes as the logarithm (log) of the organism counts (cfu/ml), then adding log reductions for the individual components and comparing to the log reduction for the mixture. This method for determining synergy has been used by others, such as disclosed in U.S. Pat. Nos. 5,019,096; 5,043,176; and 6,846,846; herein incorporated by reference to the extent they do not contradict the statements herein. However, the method described by F. C. Kull et al. in Applied Microbiology, Vol. 9, pages 538-541 (1961), herein incorporated by reference to the extent it does not contradict the statements herein, is more widely accepted and is considered to be more accurate. The Kull method is referenced in U.S. Pat. Nos. 6,432,433; 7,115,641; 7,342,044; and 7,468,384, and are herein incorporated by reference to the extent they do not contradict the statements herein. Therefore, further work was done to determine the synergistic effects of 1,4-CHDM with biocides by determining and comparing their minimum inhibitory concentrations (MIC) alone and in mixtures as described by Kull (see Example 2).

TABLE 5
Comparison of 1,4-CHDM (AB) Alone to 1,4-CHDM with Other Components (Grade codes
are given for each organism after incubation for the specified number of days.)

| | A. niger | | | C. albicans | | | P. aeruginosa − | | | E. coli − | | |
|---|---|---|---|---|---|---|---|---|---|---|---|---|
| | 3 | 13 | 30 | 3 | 14 | 30 | 3 | 13 | 30 | 3 | 14 | 30 |
| Test Material | Day | Day | Day | Day | Day | Days | Days | Days | Days | Day | Day | Day |
| Broth Alone | 5 | 5 | 5 | 5 | 5 | 5 | 5 | 5 | 5 | 5 | 5 | 5 |
| 0.5% AB | | | | | | | | | | | | |
| 0.5% AB | 4 | 4 | 4 | 3 | 4 | 4 | 4 | 4 | 4 | 5 | 4 | 4 |
| 0.5% EDTA | 4 | 4 | 4 | 4 | 5 | 5 | 4 | 4 | 5 | 5 | 5 | 5 |
| 0.5% AB + 0.2% EDTA | 4 | 2 | 2 | 4 | 4 | 4 | 4 | 4 | 4 | 4 | 4 | 4 |
| 1.25% AB | | | | | | | | | | | | |
| 1.25% AB | 4 | 4 | 4 | 1 | 3 | 4 | 4 | 3 | 4 | 4 | 4 | 4 |
| 1.25% AB + 0.2% EDTA | 2 | 0 | 0 | 4 | 2 | 2 | 2 | 2 | 2 | 2 | 0 | 0 |
| 0.25% PE | 4 | 4 | 4 | 4 | 4 | 4 | 1 | 1 | 1 | 4 | 4 | 4 |
| 1.25% AB + 0.25% PE | 4 | 4 | 4 | 4 | 4 | 4 | 0 | 0 | 0 | 4 | 4 | 4 |
| 0.5% PE | 4 | 4 | 4 | 3 | 0 | 0 | 0 | 0 | 0 | 4 | 4 | 4 |
| 1.25% AB + 0.5% PE | 4 | 4 | 4 | 3 | 2 | 2 | 0 | 0 | 0 | 4 | 2 | 2 |
| 0.25% CG | 4 | 4 | 4 | 3 | 0 | 0 | 2 | 2 | 2 | 3 | 2 | 2 |
| 1.25% AB + 0.25% CG | 4 | 4 | 4 | 4 | 3 | 2 | 0 | 0 | 0 | 2 | 0 | 0 |
| 0.5% CG | 4 | 2 | 1 | 3 | 0 | 0 | 0 | 0 | 0 | 1 | 0 | 0 |
| 1.25% AB + 0.5% CG | 4 | 3 | 2 | 2 | 0 | 0 | 0 | 0 | 0 | 1.5 | 0 | 0 |
| 2.5% AB | | | | | | | | | | | | |
| 2.5% AB | 2 | 1 | 2.3 | 0 | 2 | 1 | 3 | 2 | 0 | 3 | 2 | 0 |
| 2.5% AB + 0.2% EDTA | 0 | 0 | 0 | 3 | 1 | 1 | 1 | 0 | 0 | 0 | 0 | 0 |
| 0.25% PE | 4 | 4 | 4 | 4 | 4 | 4 | 1 | 1 | 1 | 4 | 4 | 4 |
| 2.5% AB + 0.25% PE | 3 | 2 | 1 | 3 | 2 | 2 | 0 | 0 | 0 | 0 | 0 | 0 |
| 0.5% PE | 4 | 4 | 4 | 3 | 0 | 0 | 0 | 0 | 0 | 4 | 4 | 4 |
| 2.5% AB + 0.5% PE | 3 | 2 | 0 | 2 | 0 | 0 | 0 | 0 | 0 | 1.5 | 0 | 0 |
| 0.25% CG | 4 | 4 | 4 | 3 | 0 | 0 | 2 | 2 | 2 | 3 | 2 | 2 |
| 2.5% AB + 0.25% CG | 3 | 1 | 1 | 2 | 0 | 0 | 0 | 0 | 0 | 1 | 0 | 0 |
| 0.5% CG | 4 | 2 | 1 | 3 | 0 | 0 | 0 | 0 | 0 | 1 | 0 | 0 |
| 2.5% AB + 0.5% CG | 2 | 0 | 0 | 2 | 0 | 0 | 0 | 0 | 0 | 0 | 0 | 0 |

| | S. aureus + | | | S. epidermidis + | | | S. mutans + | | |
|---|---|---|---|---|---|---|---|---|---|
| | 3 | 14 | 30 | 3 | 13 | 30 | 3 | 13 | 30 |
| Test Material | Day | Day | Day | Days | Days | Days | Day | Day | Days |
| Broth Alone | 5 | 5 | 5 | 5 | 5 | 5 | 5 | 5 | 5 |
| 0.5% AB | | | | | | | | | |
| 0.5% AB | 4 | 4 | 4 | 4 | 4 | 4 | 4 | 4 | 4 |
| 0.5% EDTA | 5 | 4 | 4 | 4 | 3 | 4 | 4 | 4 | 4 |
| 0.5% AB + 0.2% EDTA | 4 | 3.3 | 3.3 | 4 | 3.3 | 3.3 | 0 | 0 | 0 |
| 1.25% AB | | | | | | | | | |
| 1.25% AB | 3 | 2.7 | 4 | 2 | 0.7 | 2 | 4 | 2 | 2 |
| 1.25% AB + 0.2% EDTA | 2 | 0 | 0 | 2 | 0 | 0 | 0 | 0 | 0 |
| 0.25% PE | 4 | 4 | 4 | 4 | 4 | 4 | 4 | 4 | 4 |
| 1.25% AB + 0.25% PE | 4 | 3 | 3 | 4 | 3 | 4 | 4 | 4 | 4 |
| 0.5% PE | 4 | 2 | 2 | 4 | 1 | 1 | 4 | 4 | 4 |
| 1.25% AB + 0.5% PE | 0 | 0 | 0 | 0 | 0 | 0 | 0 | 0 | 0 |
| 0.25% CG | 4 | 2 | 2 | 4 | 1 | 1 | 0 | 0 | 0 |
| 1.25% AB + 0.25% CG | 0 | 0 | 0 | 0 | 0 | 0 | 0 | 0 | 0 |
| 0.5% CG | 3 | 0 | 0 | 2 | 0 | 0 | 0 | 0 | 0 |
| 1.25% AB + 0.5% CG | 0 | 0 | 0 | 0 | 0 | 0 | 0 | 0 | 0 |
| 2.5% AB | | | | | | | | | |
| 2.5% AB | 1.7 | 1 | 0 | 0.7 | 0 | 0 | 2 | 0 | 0 |
| 2.5% AB + 0.2% EDTA | 0 | 0 | 0 | 0 | 0 | 0 | 0 | 0 | 0 |
| 0.25% PE | 4 | 4 | 4 | 4 | 4 | 4 | 4 | 4 | 4 |
| 2.5% AB + 0.25% PE | 0 | 0 | 0 | 0 | 0 | 0 | 0 | 0 | 0 |
| 0.5% PE | 4 | 2 | 2 | 4 | 1 | 1 | 4 | 4 | 4 |
| 2.5% AB + 0.5% PE | 0 | 0 | 0 | 0 | 0 | 0 | 0 | 0 | 0 |
| 0.25% CG | 4 | 2 | 2 | 4 | 1 | 1 | 0 | 0 | 0 |
| 2.5% AB + 0.25% CG | 0 | 0 | 0 | 0 | 0 | 0 | 0 | 0 | 0 |
| 0.5% CG | 3 | 0 | 0 | 2 | 0 | 0 | 0 | 0 | 0 |
| 2.5% AB + 0.5% CG | 0 | 0 | 0 | 0 | 0 | 0 | 0 | 0 | 0 |

| | B. subtilis + | | | B. cepacia − | | | P. vulgaris − | | | Aeromonas sp − | | |
|---|---|---|---|---|---|---|---|---|---|---|---|---|
| | 3 | 14 | 30 | 3 | 14 | 30 | 3 | 13 | 30 | 3 | 14 | 30 |
| Test Material | Day | Day | Days | Days | Days | Days | Days | Days | Day | Days | Days | Days |
| Broth Alone | 5 | 5 | 5 | 5 | 5 | 5 | 5 | 5 | 5 | 5 | 5 | 5 |
| 0.5% AB | | | | | | | | | | | | |
| 0.5% AB | 4 | 4 | 4 | 4 | 4 | 4 | 4 | 4 | 4 | 5 | 4 | 4 |
| 0.5% EDTA | 4 | 4 | 4 | 4 | 4 | 4 | 5 | 4 | 4 | 5 | 4 | 4 |
| 0.5% AB + 0.2% EDTA | 4 | 4 | 4 | 4 | 2 | 3 | 4 | 3.7 | 3.7 | 3.3 | 2 | 2 |
| 1.25% AB | | | | | | | | | | | | |
| 1.25% AB | 4 | 4 | 4 | 4 | 2 | 4 | 3 | 2 | 0 | 4 | 3 | 2 |
| 1.25% AB + 0.2% EDTA | 3 | 2 | 2 | 2 | 0 | 0 | 2 | 0 | 0 | 2 | 1 | 1 |
| 0.25% PE | 3 | 2 | 2 | 2 | 0 | 0 | 4 | 3 | 3 | 4 | 4 | 4 |
| 1.25% AB + 0.25% PE | 2 | 4 | 4 | 0 | 0 | 0 | 4 | 2 | 2 | 4 | 2 | 2 |
| 0.5% PE | 2 | 0 | 0 | 2.5 | 0.5 | 0 | 3 | 1 | 1 | 4 | 3 | 1 |
| 1.25% AB + 0.5% PE | 2 | 0 | 0 | 0 | 0 | 0 | 3 | 2.5 | 3 | 3 | 2 | 1 |
| 0.25% CG | 4 | 4 | 4 | 2 | 0.5 | 0.5 | 4 | 2 | 2 | 4 | 4 | 4 |
| 1.25% AB + 0.25% CG | 3 | 2 | 2 | 1 | 0 | 0 | 4 | 3 | 3 | 4 | 4 | 4 |
| 0.5% CG | 3 | 1 | 1 | 2 | 0 | 0 | 3 | 2 | 2 | 4 | 4 | 4 |
| 1.25% AB + 0.5% CG | 0 | 0 | 0 | 1 | 0 | 0 | 3 | 2 | 1 | 4 | 3.5 | 2 |
| 2.5% AB | | | | | | | | | | | | |
| 2.5% AB | 3 | 2 | 1 | 0 | 0 | 0 | 1 | 0 | 0 | 3 | 1 | 0 |
| 2.5% AB + 0.2% EDTA | 1 | 0 | 0 | 1 | 0 | 0 | 0 | 0 | 0 | 2 | 0 | 0 |
| 0.25% PE | 3 | 2 | 2 | 2 | 0 | 0 | 4 | 3 | 3 | 4 | 4 | 4 |
| 2.5% AB + 0.25% PE | 4 | 4 | 4 | 0 | 0 | 0 | 2 | 1 | 1 | 2 | 0 | 0 |
| 0.5% PE | 2 | 0 | 0 | 2.5 | 0.5 | 0 | 3 | 1 | 1 | 4 | 3 | 1 |
| 2.5% AB + 0.5% PE | 4 | 4 | 3 | 0 | 0 | 0 | 4 | 2 | 1 | 1 | 0 | 0 |
| 0.25% CG | 4 | 4 | 4 | 2 | 0.5 | 0.5 | 4 | 2 | 2 | 4 | 4 | 4 |
| 2.5% AB + 0.25% CG | 2 | 1 | 1 | 0 | 0 | 0 | 2 | 1 | 1 | 3 | 2 | 2 |
| 0.5% CG | 3 | 1 | 1 | 2 | 0 | 0 | 3 | 2 | 2 | 4 | 4 | 4 |
| 2.5% AB + 0.5% CG | 0 | 0 | 0 | 0 | 0 | 0 | 2 | 1 | 0 | 1 | 0 | 0 |

| | Pathogenic Fungi | | | | | | | | |
|---|---|---|---|---|---|---|---|---|---|
| | M. canis | | | T. ruburm | | | M. furfur | | |
| | 3 | 14 | 30 | 3 | 14 | 30 | 3 | 14 | 30 |
| Test Material | Days | Days | Days | Days | Days | Days | Days | Days | Days |
| Broth Alone | 2 | 1 | 1 | 1 | 0 | 3 | 0 | 3 | 3 |
| 0.5% AB | | | | | | | | | |
| 0.5% AB | 2 | 1 | 1 | 1 | 1 | 1 | 0 | 1 | 2 |
| 0.5% EDTA | 2 | 1 | 0 | 1 | 0 | 0 | 0 | 0 | 0 |
| 0.5% AB + 0.2% EDTA | NT | NT | NT | NT | NT | NT | NT | NT | NT |
| 1.25% AB | | | | | | | | | |
| 1.25% AB | 2 | 1 | 1 | 0 | 0 | 0 | 0 | 0 | 0 |
| 1.25% AB + 0.2% EDTA | NT | NT | NT | NT | NT | NT | NT | NT | NT |
| 0.25% PE | 2 | 1 | 0 | 0 | 0 | 0 | 0 | 0 | 0 |
| 1.25% AB + 0.25% PE | 0 | 0 | 0 | 0 | 0 | 0 | 0 | 0 | 0 |
| 0.5% PE | 0 | 0 | 0 | 0 | 0 | 0 | 0 | 0 | 0 |
| 1.25% AB + 0.5% PE | 0 | 0 | 0 | 0 | 0 | 0 | 0 | 0 | 0 |
| 0.25% CG | 0 | 0 | 0 | 0 | 0 | 0 | 0 | 0 | 0 |
| 1.25% AB + 0.25% CG | 0 | 1 | 0 | 1 | 0 | 0 | 0 | 0 | 0 |
| 0.5% CG | 0 | 0 | 0 | 0 | 0 | 0 | 0 | 0 | 0 |
| 1.25% AB + 0.5% CG | 0 | 0 | 0 | 0 | 0 | 0 | 0 | 0 | 0 |
| 2.5% AB | | | | | | | | | |
| 2.5% AB | 2 | 0 | 0 | 0 | 0 | 0 | 0 | 0 | 0 |
| 2.5% AB + 0.2% EDTA | NT | NT | NT | NT | NT | NT | NT | NT | NT |
| 0.25% PE | 2 | 1 | 0 | 0 | 0 | 0 | 0 | 0 | 0 |
| 2.5% AB + 0.25% PE | 0 | 0 | 0 | 0 | 0 | 0 | 0 | 0 | 0 |
| 0.5% PE | 0 | 0 | 0 | 0 | 0 | 0 | 0 | 0 | 0 |
| 2.5% AB + 0.5% PE | 0 | 0 | 0 | 0 | 0 | 0 | 0 | 0 | 0 |
| 0.25% CG | 0 | 0 | 0 | 0 | 0 | 0 | 0 | 0 | 0 |
| 2.5% AB + 0.25% CG | 0 | 0 | 0 | 0 | 0 | 0 | 0 | 0 | 0 |
| 0.5% CG | 0 | 0 | 0 | 0 | 0 | 0 | 0 | 0 | 0 |
| 2.5% AB + 0.5% CG | 0 | 0 | 0 | 1 | 0 | 0 | 0 | 0 | 0 |

AB: Verityl ™ AB-1000 active (1,4-CHDM)
EDTA: disodium EDTA dihydrate
PE: phenoxyethanol
CG: caprylyl glycol (1,2-octanediol)

TABLE 6
Comparison of 1,4-CHDM, TMCD, and 1,3-CHDM with BIT (Grade codes are given
for each organism after incubation for the specified number of days.)

| | A. niger | | | C. albicans | | | P. aeruginosa − | | |
|---|---|---|---|---|---|---|---|---|---|
| | 3 | 13 | 30 | 3 | 14 | 30 | 3 | 13 | 30 |
| Test Material | Day | Days | Days | Days | Day | Day | Days | Days | Days |
| 0.5% 1,4-CHDM | 4 | 4 | 4 | 3 | 4 | 4 | 4 | 4 | 4 |
| 0.05% BIT | 4 | 4 | 4 | 4 | 4 | 4 | 4 | 4 | 4 |
| 0.5% 1,4-CHDM + 0.05% BIT | 4 | 4 | 4 | 3 | 0 | 0 | 4 | 2 | 2 |
| 0.2% BIT | 4 | 4 | 4 | 4 | 3 | 3 | 3 | 4 | 4 |
| 0.5% 1,4-CHDM + 0.2% BIT | 2.7 | 1 | 3 | 3 | 2 | 2 | 0 | 0 | 0 |
| 1.25% 1,4-CHDM | 4 | 4 | 4 | 1 | 3 | 4 | 4 | 3 | 4 |
| 0.05% BIT | 4 | 4 | 4 | 4 | 4 | 4 | 4 | 4 | 4 |
| 1.25% 1,4-CHDM + 0.05% BIT | 4 | 4 | 4 | 2.3 | 0 | 0 | 3 | 2 | 2 |
| 0.2% BIT | 4 | 4 | 4 | 4 | 3 | 3 | 3 | 4 | 4 |
| 1.25% 1,4-CHDM + 0.2% BIT | 1 | 0 | 0 | 3 | 0 | 0 | 0 | 0 | 0 |
| 2.5% 1,4-CHDM | 2 | 1 | 2.3 | 0 | 2 | 1 | 3 | 2 | 0 |
| 0.05% BIT | 4 | 4 | 4 | 4 | 4 | 4 | 4 | 4 | 4 |
| 1,4-CHDM 2.5% + 0.05% BIT | 1 | 0 | 0 | 2 | 0 | 0 | 2 | 0 | 0 |
| 0.2% BIT | 4 | 4 | 4 | 4 | 3 | 3 | 3 | 4 | 4 |
| 1,4-CHDM 2.5% + 0.2% BIT | 0 | 0 | 0 | 2 | 0 | 0 | 0 | 0 | 0 |
| 0.5% TMCD | 5 | 5 | 5 | 4 | 4 | 4 | 4 | 4 | 4 |
| 0.05% BIT | 4 | 4 | 4 | 4 | 4 | 4 | 4 | 4 | 4 |
| TMCD 0.5% + 0.05% BIT | 4 | 4 | 4 | 4 | 4 | 4 | 4 | 4 | 4 |
| 0.2% BIT | 4 | 4 | 4 | 4 | 3 | 3 | 3 | 4 | 4 |
| TMCD 0.5% + 0.2% BIT | 2 | 1 | 1 | 4 | 3 | 3 | 4 | 4 | 4 |
| 1.25% TMCD | 4 | 4 | 4 | 3 | 4 | 4 | 4 | 4 | 4 |
| 0.05% BIT | 4 | 4 | 4 | 4 | 4 | 4 | 4 | 4 | 4 |
| TMCD 1.25% + 0.05% BIT | 4 | 4 | 4 | 4 | 4 | 4 | 4 | 2 | 2 |
| 0.2% BIT | 4 | 4 | 4 | 4 | 3 | 3 | 3 | 4 | 4 |
| TMCD 1.25% + 0.2% BIT | 0 | 0 | 0 | 4 | 2 | 2 | 2 | 2 | 2 |
| 2.5% TMCD | 3 | 2 | 3 | 3 | 2 | 2 | 4 | 3 | 4 |
| 0.05% BIT | 4 | 4 | 4 | 4 | 4 | 4 | 4 | 4 | 4 |
| TMCD 2.5% + 0.05% BIT | 2 | 0 | 0 | 4 | 2 | 2 | 1 | 0 | 0 |
| 0.2% BIT | 4 | 4 | 4 | 4 | 3 | 3 | 3 | 4 | 4 |
| TMCD 2.5% + 0.2% BIT | 0 | 0 | 0 | 3 | 0 | 0 | 2 | 1 | 1 |
| 0.5% 1,3-CHDM | 5 | 5 | 5 | 4 | 4 | 4 | 5 | 5 | 5 |
| 0.05% BIT | 4 | 4 | 4 | 4 | 4 | 4 | 4 | 4 | 4 |
| 1,3-CHDM 0.5% + 0.05% BIT | 4 | 4 | 4 | 4 | 4 | 4 | 4 | 4 | 4 |
| 0.2% BIT | 4 | 4 | 4 | 4 | 3 | 3 | 3 | 4 | 4 |
| 1,3-CHDM 0.5% + 0.2% BIT | 4 | 2 | 2 | 4 | 4 | 4 | 3 | 0 | 0 |
| 1.25% 1,3-CHDM | 5 | 4 | 4 | 4 | 4 | 4 | 4 | 4 | 4 |
| 0.05% BIT | 4 | 4 | 4 | 4 | 4 | 4 | 4 | 4 | 4 |
| 1,3-CHDM 1.25% + 0.05% BIT | 4 | 4 | 4 | 4 | 4 | 4 | 4 | 4 | 4 |
| 0.2% BIT | 4 | 4 | 4 | 4 | 3 | 3 | 3 | 4 | 4 |
| 1,3-CHDM 1.25% + 0.2% BIT | 2.3 | 2 | 2 | 4 | 4 | 4 | 2 | 0 | 0 |
| 2.5% 1,3-CHDM | 4 | 4 | 4 | 4 | 4 | 4 | 4 | 4 | 4 |
| 0.05% BIT | 4 | 4 | 4 | 4 | 4 | 4 | 4 | 4 | 4 |
| 1,3-CHDM 2.5% + 0.05% BIT | 4 | 4 | 4 | 4 | 4 | 4 | 4 | 4 | 4 |
| 0.2% BIT | 4 | 4 | 4 | 4 | 3 | 3 | 3 | 4 | 4 |
| 1,3-CHDM 2.5% + 0.2% BIT | 2 | 1 | 1 | 4 | 3 | 3 | 1 | 0 | 0 |
| | E. coli − | | | S. aureus + | | | S. epidermidis + | | |
| | 3 | 14 | 30 | 3 | 14 | 30 | 3 | 13 | 30 |
| Test Material | Day | Day | Day | Day | Day | Day | Days | Days | Days |
| 0.5% 1,4-CHDM | 5 | 4 | 4 | 4 | 4 | 4 | 4 | 4 | 4 |
| 0.05% BIT | 4 | 4 | 4 | 4 | 4 | 4 | 4 | 4 | 4 |
| 0.5% 1,4-CHDM + 0.05% BIT | 0 | 0 | 0 | 4 | 4 | 4 | 4 | 4 | 4 |
| 0.2% BIT | 1 | 0 | 0 | 4 | 3 | 3 | 4 | 2 | 2 |
| 0.5% 1,4-CHDM + 0.2% BIT | 2 | 0 | 0 | 4 | 4 | 4 | 4 | 4 | 4 |
| 1.25% 1,4-CHDM | 4 | 4 | 4 | 3 | 2.7 | 4 | 2 | 0.7 | 2 |
| 0.05% BIT | 4 | 4 | 4 | 4 | 4 | 4 | 4 | 4 | 4 |
| 1.25% 1,4-CHDM + 0.05% BIT | 0 | 0 | 0 | 4 | 4 | 4 | 4 | 2 | 2 |
| 0.2% BIT | 1 | 0 | 0 | 4 | 3 | 3 | 4 | 2 | 2 |
| 1.25% 1,4-CHDM + 0.2% BIT | 1 | 0 | 0 | 4 | 4 | 4 | 4 | 3 | 3 |
| 2.5% 1,4-CHDM | 3 | 2 | 0 | 1.7 | 1 | 0 | 0.7 | 0 | 0 |
| 0.05% BIT | 4 | 4 | 4 | 4 | 4 | 4 | 4 | 4 | 4 |
| 1,4-CHDM 2.5% + 0.05% BIT | 0 | 0 | 0 | 4 | 2 | 2 | 3 | 1.3 | 1.3 |
| 0.2% BIT | 1 | 0 | 0 | 4 | 3 | 3 | 4 | 2 | 2 |
| 1,4-CHDM 2.5% + 0.2% BIT | 0 | 0 | 0 | 1.3 | 0 | 0 | 0 | 0 | 0 |
| 0.5% TMCD | 5 | 4 | 4 | 4 | 4 | 4 | 4 | 4 | 4 |
| 0.05% BIT | 4 | 4 | 4 | 4 | 4 | 4 | 4 | 4 | 4 |
| TMCD 0.5% + 0.05% BIT | 2 | 1 | 1 | 3 | 1.7 | 1.7 | 2 | 1 | 1 |
| 0.2% BIT | 1 | 0 | 0 | 4 | 3 | 3 | 4 | 2 | 2 |
| TMCD 0.5% + 0.2% BIT | 1 | 0 | 0 | 4 | 4 | 4 | 4 | 4 | 4 |
| 1.25% TMCD | 4 | 4 | 4 | 4 | 4 | 4 | 2 | 3 | 4 |
| 0.05% BIT | 4 | 4 | 4 | 4 | 4 | 4 | 4 | 4 | 4 |
| TMCD 1.25% + 0.05% BIT | 1 | 0 | 0 | 1 | 0 | 0 | 1 | 0 | 0 |
| 0.2% BIT | 1 | 0 | 0 | 4 | 3 | 3 | 4 | 2 | 2 |
| TMCD 1.25% + 0.2% BIT | 1 | 0 | 0 | 4 | 4 | 4 | 4 | 4 | 4 |
| 2.5% TMCD | 4 | 4 | 4 | 2 | 1 | 1 | 2 | 2 | 2 |
| 0.05% BIT | 4 | 4 | 4 | 4 | 4 | 4 | 4 | 4 | 4 |
| TMCD 2.5% + 0.05% BIT | 0 | 0 | 0 | 0 | 0 | 0 | 0 | 0 | 0 |
| 0.2% BIT | 1 | 0 | 0 | 4 | 3 | 3 | 4 | 2 | 2 |
| TMCD 2.5% + 0.2% BIT | 0 | 0 | 0 | 0 | 0 | 0 | 0 | 0 | 0 |
| 0.5% 1,3-CHDM | 5 | 5 | 5 | 4 | 4 | 4 | 4 | 4 | 4 |
| 0.05% BIT | 4 | 4 | 4 | 4 | 4 | 4 | 4 | 4 | 4 |
| 1,3-CHDM 0.5% + 0.05% BIT | 2 | 2 | 2 | 4 | 4 | 4 | 4 | 4 | 4 |
| 0.2% BIT | 1 | 0 | 0 | 4 | 3 | 3 | 4 | 2 | 2 |
| 1,3-CHDM 0.5% + 0.2% BIT | 3.7 | 0 | 0 | 4 | 4 | 4 | 4 | 4 | 4 |
| 1.25% 1,3-CHDM | 5 | 4 | 4 | 4 | 4 | 4 | 4 | 3.7 | 4 |
| 0.05% BIT | 4 | 4 | 4 | 4 | 4 | 4 | 4 | 4 | 4 |
| 1,3-CHDM 1.25% + 0.05% BIT | 2 | 2 | 2 | 4 | 4 | 4 | 4 | 4 | 4 |
| 0.2% BIT | 1 | 0 | 0 | 4 | 3 | 3 | 4 | 2 | 2 |
| 1,3-CHDM 1.25% + 0.2% BIT | 3 | 0 | 0 | 4 | 4 | 4 | 4 | 4 | 4 |
| 2.5% 1,3-CHDM | 5 | 4 | 4 | 4 | 4 | 4 | 4 | 3 | 4 |
| 0.05% BIT | 4 | 4 | 4 | 4 | 4 | 4 | 4 | 4 | 4 |
| 1,3-CHDM 2.5% + 0.05% BIT | 2 | 2 | 2 | 4 | 3 | 3 | 4 | 3 | 3 |
| 0.2% BIT | 1 | 0 | 0 | 4 | 3 | 3 | 4 | 2 | 2 |
| 1,3-CHDM 2.5% + 0.2% BIT | 3 | 0 | 0 | 4 | 4 | 4 | 4 | 4 | 4 |
| | S. mutans + | | | B. subtilis + | | | B. cepacia − | | |
| | 3 | 13 | 30 | 3 | 14 | 30 | 3 | 14 | 30 |
| Test Material | Day | Day | Day | Day | Day | Day | Day | Day | Day |
| 0.5% 1,4-CHDM | 4 | 4 | 4 | 4 | 4 | 4 | 4 | 4 | 4 |
| 0.05% BIT | 1.7 | 0 | 0 | 4 | 4 | 4 | 4 | 4 | 4 |
| 0.5% 1,4-CHDM + 0.05% BIT | 2 | 0 | 0 | 4 | 2 | 2 | 4 | 4 | 4 |
| 0.2% BIT | 0 | 0 | 0 | 4 | 4 | 4 | 3 | 1 | 2 |
| 0.5% 1,4-CHDM + 0.2% BIT | 0 | 0 | 0 | 2.7 | 2 | 2 | 0 | 0 | 0 |
| 1.25% 1,4-CHDM | 4 | 2 | 2 | 4 | 4 | 4 | 4 | 2 | 4 |
| 0.05% BIT | 1.7 | 0 | 0 | 4 | 4 | 4 | 4 | 4 | 4 |
| 1.25% 1,4-CHDM + 0.05% BIT | 0 | 0 | 0 | 2 | 1 | 1 | 2 | 2 | 1 |
| 0.2% BIT | 0 | 0 | 0 | 4 | 4 | 4 | 3 | 1 | 2 |
| 1.25% 1,4-CHDM + 0.2% BIT | 0 | 0 | 0 | 3 | 1 | 1 | 0 | 0 | 0 |
| 2.5% 1,4-CHDM | 2 | 0 | 0 | 3 | 2 | 1 | 0 | 0 | 0 |
| 0.05% BIT | 1.7 | 0 | 0 | 4 | 4 | 4 | 4 | 4 | 4 |
| 1,4-CHDM 2.5% + 0.05% BIT | 0 | 0 | 0 | 2 | 0 | 0 | 2 | 1 | 0 |
| 0.2% BIT | 0 | 0 | 0 | 4 | 4 | 4 | 3 | 1 | 2 |
| 1,4-CHDM 2.5% + 0.2% BIT | 0 | 0 | 0 | 2 | 0 | 0 | 0 | 0 | 0 |
| 0.5% TMCD | 4 | 4 | 4 | 4 | 4 | 4 | 4 | 4 | 4 |
| 0.05% BIT | 1.7 | 0 | 0 | 4 | 4 | 4 | 4 | 4 | 4 |
| TMCD 0.5% + 0.05% BIT | 0 | 0 | 0 | 4 | 4 | 4 | 4 | 4 | 4 |
| 0.2% BIT | 0 | 0 | 0 | 4 | 4 | 4 | 3 | 1 | 2 |
| TMCD 0.5% + 0.2% BIT | 0 | 0 | 0 | 4 | 2 | 2 | 2 | 1 | 3 |
| 1.25% TMCD | 4 | 4 | 4 | 4 | 4 | 4 | 3 | 2 | 4 |
| 0.05% BIT | 1.7 | 0 | 0 | 4 | 4 | 4 | 4 | 4 | 4 |
| TMCD 1.25% + 0.05% BIT | 0 | 0 | 0 | 4 | 4 | 4 | 4 | 3 | 3 |
| 0.2% BIT | 0 | 0 | 0 | 4 | 4 | 4 | 3 | 1 | 2 |
| TMCD 1.25% + 0.2% BIT | 0 | 0 | 0 | 2 | 1 | 1 | 1 | 0 | 0 |
| 2.5% TMCD | 3 | 1 | 0 | 4 | 4 | 4 | 1 | 0 | 0 |
| 0.05% BIT | 1.7 | 0 | 0 | 4 | 4 | 4 | 4 | 4 | 4 |
| TMCD 2.5% + 0.05% BIT | 0 | 0 | 0 | 3 | 2 | 2 | 1 | 1 | 1.7 |
| 0.2% BIT | 0 | 0 | 0 | 4 | 4 | 4 | 3 | 1 | 2 |
| TMCD 2.5% + 0.2% BIT | 0 | 0 | 0 | 2 | 0 | 0 | 1 | 0 | 0 |
| 0.5% 1,3-CHDM | 5 | 4 | 4 | 4 | 4 | 4 | 5 | 5 | 5 |
| 0.05% BIT | 1.7 | 0 | 0 | 4 | 4 | 4 | 4 | 4 | 4 |
| 1,3-CHDM 0.5% + 0.05% BIT | 0 | 0 | 0 | 4 | 4 | 4 | 4 | 4 | 4 |
| 0.2% BIT | 0 | 0 | 0 | 4 | 4 | 4 | 3 | 1 | 2 |
| 1,3-CHDM 0.5% + 0.2% BIT | 0 | 0 | 0 | 4 | 4 | 4 | 3 | 1 | 2.7 |
| 1.25% 1,3-CHDM | 4 | 4 | 4 | 4 | 4 | 4 | 4.7 | 4.7 | 4.7 |
| 0.05% BIT | 1.7 | 0 | 0 | 4 | 4 | 4 | 4 | 4 | 4 |
| 1,3-CHDM 1.25% + 0.05% BIT | 0 | 0 | 0 | 4 | 4 | 4 | 4 | 4 | 4 |
| 0.2% BIT | 0 | 0 | 0 | 4 | 4 | 4 | 3 | 1 | 2 |
| 1,3-CHDM 1.25% + 0.2% BIT | 2 | 0 | 0 | 4 | 4 | 4 | 2 | 1 | 3 |
| 2.5% 1,3-CHDM | 4 | 4 | 4 | 4 | 4 | 4 | 4 | 4 | 4 |
| 0.05% BIT | 1.7 | 0 | 0 | 4 | 4 | 4 | 4 | 4 | 4 |
| 1,3-CHDM 2.5% + 0.05% BIT | 0 | 0 | 0 | 4 | 4 | 4 | 4 | 3 | 4 |
| 0.2% BIT | 0 | 0 | 0 | 4 | 4 | 4 | 3 | 1 | 2 |
| 1,3-CHDM 2.5% + 0.2% BIT | 0 | 0 | 0 | 4 | 3 | 3 | 1 | 0 | 0 |

| | P. vulqaris − | | | Aeromonas sp − | | |
|---|---|---|---|---|---|---|
| | 3 | 13 | 30 | 3 | 14 | 30 |
| Test Material | Day | Day | Day | Days | Days | Days |
| 0.5% 1,4-CHDM | 4 | 4 | 4 | 5 | 4 | 4 |
| 0.05% BIT | 4 | 4 | 4 | 5 | 4 | 4 |
| 0.5% 1,4-CHDM + 0.05% BIT | 3 | 2 | 2 | 4 | 4 | 4 |
| 0.2% BIT | 4 | 2 | 0 | 4 | 2 | 2 |
| 0.5% 1,4-CHDM + 0.2% BIT | 1 | 0 | 0 | 3 | 1 | 1 |
| 1.25% 1,4-CHDM | 3 | 2 | 0 | 4 | 3 | 2 |
| 0.05% BIT | 4 | 4 | 4 | 5 | 4 | 4 |
| 1.25% 1,4-CHDM + 0.05% BIT | 3 | 2 | 2 | 4 | 3.3 | 3.3 |
| 0.2% BIT | 4 | 2 | 0 | 4 | 2 | 2 |
| 1.25% 1,4-CHDM + 0.2% BIT | 0 | 0 | 0 | 1 | 0 | 0 |
| 2.5% 1,4-CHDM | 1 | 0 | 0 | 3 | 1 | 0 |
| 0.05% BIT | 4 | 4 | 4 | 5 | 4 | 4 |
| 1,4-CHDM 2.5% + 0.05% BIT | 1 | 0 | 0 | 3 | 1.3 | 1 |
| 0.2% BIT | 4 | 2 | 0 | 4 | 2 | 2 |
| 1,4-CHDM 2.5% + 0.2% BIT | 0 | 0 | 0 | 1 | 0 | 0 |
| 0.5% TMCD | 4 | 4 | 4 | 5 | 4 | 4 |
| 0.05% BIT | 4 | 4 | 4 | 5 | 4 | 4 |
| TMCD 0.5% + 0.05% BIT | 4 | 2 | 2 | 4 | 4 | 4 |
| 0.2% BIT | 4 | 2 | 0 | 4 | 2 | 2 |
| TMCD 0.5% + 0.2% BIT | 2 | 0 | 0 | 3 | 1 | 1 |
| 1.25% TMCD | 3 | 2 | 0 | 4 | 4 | 4 |
| 0.05% BIT | 4 | 4 | 4 | 5 | 4 | 4 |
| TMCD 1.25% + 0.05% BIT | 2.7 | 2 | 2 | 4 | 4 | 4 |
| 0.2% BIT | 4 | 2 | 0 | 4 | 2 | 2 |
| TMCD 1.25% + 0.2% BIT | 1 | 0 | 0 | 3 | 1 | 1 |
| 2.5% TMCD | 4 | 1 | 0 | 4 | 2 | 3 |
| 0.05% BIT | 4 | 4 | 4 | 5 | 4 | 4 |
| TMCD 2.5% + 0.05% BIT | 1 | 0 | 0 | 4 | 2 | 2 |
| 0.2% BIT | 4 | 2 | 0 | 4 | 2 | 2 |
| TMCD 2.5% + 0.2% BIT | 0 | 0 | 0 | 2 | 0 | 0 |
| 0.5% 1,3-CHDM | 5 | 5 | 5 | 5 | 4 | 4 |
| 0.05% BIT | 4 | 4 | 4 | 5 | 4 | 4 |
| 1,3-CHDM 0.5% + 0.05% BIT | 4 | 4 | 4 | 5 | 4 | 4 |
| 0.2% BIT | 4 | 2 | 0 | 4 | 2 | 2 |
| 1,3-CHDM 0.5% + 0.2% BIT | 3 | 0 | 0 | 3 | 1 | 1 |
| 1.25% 1,3-CHDM | 5 | 4 | 4 | 4 | 4 | 4 |
| 0.05% BIT | 4 | 4 | 4 | 5 | 4 | 4 |
| 1,3-CHDM 1.25% + 0.05% BIT | 4 | 4 | 4 | 4 | 4 | 4 |
| 0.2% BIT | 4 | 2 | 0 | 4 | 2 | 2 |
| 1,3-CHDM 1.25% + 0.2% BIT | 2 | 0 | 0 | 3 | 1 | 1 |
| 2.5% 1,3-CHDM | 4 | 4 | 4 | 4 | 4 | 4 |
| 0.05% BIT | 4 | 4 | 4 | 5 | 4 | 4 |
| 1,3-CHDM 2.5% + 0.05% BIT | 4 | 2 | 2 | 4 | 4 | 4 |
| 0.2% BIT | 4 | 2 | 0 | 4 | 2 | 2 |
| 1,3-CHDM 2.5% + 0.2% BIT | 2 | 0 | 0 | 3 | 1 | 1 |

BIT: Benzisothiazolinone

### Example 2

### Synergistic Activity Assessment

### Preparation of Inoculum

The test cultures are listed in Table 7 along with the incubation temperatures used for growth and minimum inhibitory concentration (MIC) testing. E. coli, S. aureus, and P. aeruginosa were cultured in Trypticase-soy broth (TSB) for 20-28 hours for preparation of inocula. C. albicans was cultured in Sabouraud dextrose broth (SDB) for approximately 44-52 hours for preparation of inoculum. A. niger was cultured on Sabouraud dextrose agar (SDA) for 3-4 days until there was confluent growth and visible spore formation. Spores were harvested from the SDA plates by flooding the surface of the plate with 5-10 mL of phosphate-buffered saline (PBS) and gently spreading the liquid across the surface of the plate with a sterile T-shaped plastic spreader (Copan Diagnostics) until there was a well-mixed suspension of spores. The resulting spore suspension was collected using a serological pipette and stored at 2-8° C. until use.

Inoculum concentration was determined by dilution plating of the cultures or spore suspension. A serial dilution in PBS was made to 10⁻⁸ for the bacterial cultures or 10⁻⁵ for the fungal cultures. Fifty or 100 μL of the final dilution was spread on two Trypticase-soy agar (TSA) plates for bacteria or two SDA plates for fungi. Plates were incubated at the temperatures listed for the respective organism listed in Table 7. After 24 to 48 hours plates were counted and the concentrations used in each experiment were calculated.

The A. niger spore suspensions were concentrated to a level of 1-2×10⁸ spores/mL by centrifugation and resuspension in a portion of the resulting supernatant.

TABLE 7
Test organisms used in synergy study

| | Incubation |
|---|---|
| Organism (Genus and species and | temperature |
| strain) source | (° C. ±2° C.) |
| Escherichia coli ATCC 25922 | 35 |
| Candida albicans ATCC 10231 | 25 |
| Aspergillus niger ATCC 16404 | 25 |
| Staphylococcus aureus ATCC 25923 | 35 |
| Pseudomonas aeruginosa ATCC 27853 | 30 |

### Preparation of CHDM and Antimicrobial Agents

The antimicrobial agents tested for synergy along with the respective diluents and working stock solution concentrations are shown below. For determination of MIC ranges, individual antimicrobial agent stock solutions were added to sterile medium to yield the highest level test concentrations, and then serially diluted in the medium to prepare the range of test concentrations. For synergy testing individual test concentrations were prepared in sterile medium, and then blended to form the desired combinations.

TABLE 8
Antimicrobial agents tested in combination with 1,4-CHDM for synergy

| | | Stock conc. |
|---|---|---|
| Antimicrobial Agent | Diluent | (wt %) |
| 1,4-cyclohexane dimethanol (CHDM) | Water | 20, 60 |
| Phenoxyethanol (PE) | None | 100 |
| Caprylyl Glycol (CG) (1,2-octanediol) | Ethanol:water | 14 |
| | (94:6) | |
| Methylparaben (MP) | Ethanol | 20 |
| Methylisothiazolinone (MIT) | Water | 9.6 |
| 9:1 wt ratio Benzyl Alcohol (BA) and | None | 100 |
| Dehydroacetic Acid (DHA) | | |
| Chlorphenesin (CP) | Ethanol | 20 |
| DMDM Hydantoin (DMDMH) | Water | 4, 20 |
| Iodopropynyl butylcarbamate (IPBC) | Ethanol | 10 |
| Benzisothiazolinone (BIT) | Water | 9 |
| MIT:BIT (1:1 mixture, w:w) | Water | 0.5 total |

### MIC Determination for Individual Antimicrobial Agents

MICs were determined using a high-throughput microplate method. Individual antimicrobial agents were added to TSB (pH 7.3) for bacterial testing or SDB (pH 5.6) for fungal testing at the highest concentration to be tested. A serial dilution series was prepared at a dilution ratio of 1:1.3333 such that a one log range was covered in nine dilutions. Two-hundred microliters of the diluted antimicrobial agents were dispensed into four wells each of a sterile, 96-well, flat-bottom microplate (Nalge Nunc International). Four additional wells of the highest concentration were dispensed to serve as uninoculated high-level controls. Eight additional wells containing only broth medium were also prepared, four to serve as negative controls and four as positive controls. Three wells of each antimicrobial agent dilution were inoculated with one of the test strains in Table 7. The final well of each antimicrobial agent dilution was left uninoculated to serve as a control (and means of compensating) for any absorbance/turbidity due to the test compounds.

Microplates were inoculated using the cultures or spore suspensions prepared as described above. The S. aureus cultures were used undiluted, while the E. coli and P. aeruginosa cultures were used either undiluted or after a 1:2 dilution in sterile TSB. The C. albicans cultures were used either undiluted or following a 2-fold concentration by centrifugation. One of two means of inoculation was used to deliver a final concentration of approximately 10⁵ colony-forming units (CFU)/mL of C. albicans, 10⁵ spores/mL of A. niger, or 10⁶ CFU/mL of bacteria. The primary method used a stainless steel pin replicator (Nalge Nunc International) mounted on a hand-operated bench-top press (Schmidt Technology Corporation) fitted with a custom-built microplate holder to dispense one microliter of each inoculum from a “master” plate containing 50-100 μL of culture into the wells of the test plate. The pin replicator was sterilized before and between inoculations by immersing in ethanol and flaming. The alternative method of inoculation was by directly pipetting 20 μL of a 1:20 dilution of each culture or spore suspension into the appropriate wells of the test plates. This method was found to be more consistent, especially when working with fungal cultures which can settle quickly in the master plate causing variability in the number of cells/spores collected on the pins.

Inoculated test plates were covered with a sterile plate lid (Nunc, Inc.) and incubated at the temperatures listed in Table 7. Growth of organisms in the plates were measured photometrically at 650 nm after 1-4 days of incubation (1 and 2 days for bacteria, 2 and 3 days for A. niger, and 2, 3, and 4 days for C. albicans) using a microplate spectrophotometer (Molecular Devices, Inc.).

The optical density of each test well was processed by first subtracting the average reading for each uninoculated well, then comparing to a positive threshold to determine “positive” or “negative” status. The fourth well containing each antimicrobial agent dilution which was left uninoculated was used in a few cases to subtract out any contribution of the antimicrobial agent to the optical density of the test wells. The positive threshold was calculated using one of two methods. The primary method was by multiplication of the standard deviation for the negative control wells in each plate by ten. The alternative method was by using 5% of the average positive control optical density for each plate. This alternative method approximated the sensitivity of a visual determination, while the primary method was typically more sensitive than visual determination.

### Synergy Testing of Antimicrobial Agent Combinations

The individual antimicrobial agents listed in Table 8 were tested in combination with CHDM. The MIC values determined in the individual antimicrobial agent testing described above were used to establish a target MIC. In each experiment, MIC values for the individual antimicrobial agents and CHDM were determined in order to eliminate any variability due to comparison of data from different dates. Testing was done over a range of four concentrations separated by a factor of 1.3333 as described above. The four concentrations were the target MIC plus one dilution level higher and two dilution levels lower than the target. Combinations of antimicrobial agents and CHDM were made at 50% of the target MIC values and the higher and lower individual levels. Additionally, 50% level series were also tested with the antimicrobial agent or CHDM at one dilution level lower than the target level. The method of Kull et. al. (F. C. Kull et al., Applied Microbiology, 9, p 538-541 (1961)) was used to determine whether there was synergistic activity between CHDM and each antimicrobial agent for inhibition of each of the five microbes. MICs for the individually-tested CHDM and antimicrobial agent, as well as the concentration of CHDM and antimicrobial agent in the combination MIC were used to calculate a synergy index (SI) according to the equation:

SI=Q_{A}/Qₐ+Q_{B}/Q_{b}

where Qₐ and Q_{b} are the minimum inhibitory concentrations for CHDM and a antimicrobial agent, respectively, when tested independently, and Q_{A} and Q_{B} are the concentrations of CHDM and a antimicrobial agent, respectively, in combination at an inhibitory concentration. Accordingly, synergy is defined as a SI less than one.

### Results

Tables 9 through 49 provide the results for testing of the CHDM/antimicrobial agent combinations for synergistic activity. A combination was deemed to be synergistic when at least two results produced a SI<1.

Reported in Tables 9 through 49 are the organism tested, the plate number for the specific source of the data, the number of days of incubation prior to analysis of the plate, the concentration in weight percent of CHDM in each analysis (Q_{Aa}), the concentration in weight percent of the antimicrobial agent in each analysis (Q_{Bb}), the synergy index (SI), the weight ratio of the antimicrobial agent and CHDM (B/A), the concentration of CHDM in the mixture as a percentage of the CHDM-alone MIC, and the concentration of antimicrobial agent in the mixture as a percentage of the antimicrobial agent-alone MIC.

In some cases the synergy was so strong that it was not possible, within the design of the experiment, to capture the minimum concentrations of the combinations that were sufficient to inhibit the target organism while still determining the MICs for the individual components in the mixture. To establish a maximum SI in such cases, the minimum concentration mixture tested was used as the source of Q_{A} and Q_{B}. Since the actual MIC would have been lower than the value used, the actual SI would also have been even lower. Similarly, there were cases where the mixture MIC was determined, but one or both of the individual component results were positive for growth (not inhibited) even at the highest individual concentration(s) tested. Thus the maximum concentration(s) tested for the individual component(s) were used as the MIC values in the SI calculation, and again, the actual SI would have been lower than that reported.

There were eight combinations of antimicrobial agents with specific organisms for which a SI was not determined. In those cases, individual MICs were determined but the concentrations tested for the combinations were all below that required to inhibit the test organism. In each case the SI if determined would have been above 1.0. The specific combinations and organism(s) are listed below:

| Antimicrobial agent | |
|---|---|
| combination | Organism(s) |
| 1,4-CHDM and MIT | E. coli |
| 1,4-CHDM and DMDMH | E. coli, S. aureus |
| 1,4-CHDM and IPBC | C. albicans, S. aureus |
| 1,4-CHDM and BIT | A. niger, C. albicans, S. aureus |
| 1,4-CHDM and BIT:MIT | A. niger |

The following antimicrobial agents showed synergism for the indicated organism(s). Each organism is followed by the respective table number.

| Antimicrobial agent | Organism(s) |
|---|---|
| Phenoxyethanol | A. niger (9), C. albicans |
| | (10), E. coli (11), S. aureus |
| | (13) |
| Caprylyl glycol | A. niger (14), C. albicans |
| | (15) |
| Methylisothiazolinone | P. aeruginosa (26) |
| Chlorphenesin | C. albicans (29) |
| Benzyl alcohol:Dehydroacetic acid | A. niger (33), C. albicans |
| | (34), S. aureus (37) |
| DMDM hydantoin | C. albicans (39) |
| Iodopropynyl butylcarbamate | P. aeruginosa (43) |
| Benzisothiazolinone | P. aeruginosa (45) |
| Benzisothiazolinone:Methylisothiazolinone | P. aeruginosa (48) |

TABLE 9
Synergy testing of combination of Phenoxyethanol
(PE) and CHDM with A. niger

| | | | | | | | % | |
|---|---|---|---|---|---|---|---|---|
| Organism | Plate # | Day | Q_{A/a} | Q_{B/b} | SI | B/A | CHDM | % PE |
| A. niger | 122C | 2 | 2.33 | 0.00 | 1.00 | — | 100.0 | 0.0 |
| A. niger | 122C | 2 | 1.56 | 0.16 | 1.34 | 0.10 | 67.0 | 65.9 |
| A. niger | 122C | 2 | 0.66 | 0.09 | 0.66 | 0.13 | 28.3 | 37.1 |
| A. niger | 122C | 2 | 1.56 | 0.12 | 1.17 | 0.07 | 67.0 | 49.4 |
| A. niger | 122C | 2 | 0.00 | 0.24 | 1.00 | — | 0.0 | 100.0 |
| A. niger | 122C | 3 | 2.33 | 0.00 | 1.00 | — | 100.0 | 0.0 |
| A. niger | 122C | 3 | 1.17 | 0.12 | 1.00 | 0.10 | 50.2 | 49.5 |
| A. niger | 122C | 3 | 0.87 | 0.12 | 0.87 | 0.13 | 37.3 | 49.5 |
| A. niger | 122C | 3 | 1.56 | 0.12 | 1.17 | 0.07 | 67.0 | 49.5 |
| A. niger | 122C | 3 | 0.00 | 0.24 | 1.00 | — | 0.0 | 100.0 |
| A. niger | 242C | 2 | 6.40 | 0.00 | 1.00 | — | 100.0 | 0.0 |
| A. niger | 242C | 2 | 2.40 | 0.20 | 1.04 | 0.08 | 37.5 | 66.7 |
| A. niger | 242C | 2 | 1.80 | 0.20 | 0.95 | 0.11 | 28.1 | 66.7 |
| A. niger | 242C | 2 | 3.20 | 0.20 | 1.17 | 0.06 | 50.0 | 66.7 |
| A. niger | 242C | 2 | 0.00 | 0.30 | 1.00 | — | 0.0 | 100.0 |
| A. niger | 242C | 3 | 6.40 | 0.00 | 1.00 | — | 100.0 | 0.0 |
| A. niger | 242C | 3 | 2.40 | 0.20 | 0.88 | 0.08 | 37.5 | 50.0 |
| A. niger | 242C | 3 | 1.80 | 0.20 | 0.78 | 0.11 | 28.1 | 50.0 |
| A. niger | 242C | 3 | 3.20 | 0.20 | 1.00 | 0.06 | 50.0 | 50.0 |
| A. niger | 242C | 3 | 0.00 | 0.40 | 1.00 | — | 0.0 | 100.0 |

TABLE 10
Synergy testing of combination of Phenoxyethanol
(PE) and CHDM with C. albicans

| Organism | Plate # | Day | Q_{A/a} | Q_{B/b} | SI | B/A | % CHDM | % PE |
|---|---|---|---|---|---|---|---|---|
| C. albicans | 116B | 2 | 3.56 | 0.00 | 1.00 | — | 100.0 | 0.0 |
| C. albicans | 116B | 2 | 1.34 | 0.06 | 0.66 | 0.05 | 37.6 | 28.1 |
| C. albicans | 116B | 2 | 1.78 | 0.11 | 1.00 | 0.06 | 50.0 | 50.0 |
| C. albicans | 116B | 2 | 1.34 | 0.05 | 0.59 | 0.03 | 37.6 | 21.1 |
| C. albicans | 116B | 2 | 0.00 | 0.22 | 1.00 | — | 0.0 | 100.0 |
| C. albicans | 116B | 3 | 4.75 | 0.00 | 1.00 | — | 100.0 | 0.0 |
| C. albicans | 116B | 3 | 1.34 | 0.06 | 0.56 | 0.05 | 28.2 | 28.2 |
| C. albicans | 116B | 3 | 1.78 | 0.11 | 0.87 | 0.06 | 37.5 | 50.0 |
| C. albicans | 116B | 3 | 1.78 | 0.06 | 0.66 | 0.03 | 37.5 | 28.2 |
| C. albicans | 116B | 3 | 0.00 | 0.22 | 1.00 | — | 0.0 | 100.0 |
| C. albicans | 240B | 2 | 5.85 | 0.00 | 1.00 | — | 100.0 | 0.0 |
| C. albicans | 240B | 2 | 3.90 | 0.18 | 1.33 | 0.05 | 66.7 | 66.7 |
| C. albicans | 240B | 2 | 2.93 | 0.18 | 1.17 | 0.06 | 50.1 | 66.7 |
| C. albicans | 240B | 2 | 3.90 | 0.14 | 1.17 | 0.03 | 66.7 | 50.0 |
| C. albicans | 240B | 2 | 0.00 | 0.27 | 1.00 | — | 0.0 | 100.0 |
| C. albicans | 240B | 3 | 5.85 | 0.00 | 1.00 | — | 100.0 | 0.0 |
| C. albicans | 240B | 3 | 3.90 | 0.18 | 1.33 | 0.05 | 66.7 | 66.7 |
| C. albicans | 240B | 3 | 3.90 | 0.24 | 1.56 | 0.06 | 66.7 | 88.9 |
| C. albicans | 240B | 3 | 5.20 | 0.18 | 1.56 | 0.03 | 88.9 | 66.7 |
| C. albicans | 240B | 3 | 0.00 | 0.27 | 1.00 | — | 0.0 | 100.0 |
| C. albicans | 252B | 2 | 6.67 | 0.00 | 1.00 | — | 100.0 | 0.0 |
| C. albicans | 252B | 2 | 3.33 | 0.20 | 1.39 | 0.06 | 49.9 | 88.9 |
| C. albicans | 252B | 2 | 2.50 | 0.20 | 1.26 | 0.08 | 37.5 | 88.9 |
| C. albicans | 252B | 2 | 3.33 | 0.15 | 1.17 | 0.05 | 49.9 | 66.7 |
| C. albicans | 252B | 2 | 0.00 | 0.23 | 1.00 | — | 0.0 | 100.0 |
| C. albicans | 252B | 3 | 6.67 | 0.00 | 1.00 | — | 100.0 | 0.0 |
| C. albicans | 252B | 3 | 3.33 | 0.20 | 1.17 | 0.06 | 49.9 | 66.7 |
| C. albicans | 252B | 3 | 0.00 | 0.30 | 1.00 | — | 0.0 | 100.0 |

TABLE 11
Synergy testing of combination of Phenoxyethanol (PE) and CHDM with E. coli

| Organism | Plate # | Day | Q_{A/a} | Q_{B/b} | SI | B/A | % CHDM | % PE |
|---|---|---|---|---|---|---|---|---|
| E. coli | 105A | 1 | 1.30 | 0.00 | 1.00 | — | 100.0 | 0.0 |
| E. coli | 105A | 1 | 0.65 | 0.18 | 0.88 | 0.28 | 50.0 | 37.5 |
| E. coli | 105A | 1 | 0.49 | 0.18 | 0.75 | 0.37 | 37.7 | 37.5 |
| E. coli | 105A | 1 | 0.87 | 0.18 | 1.04 | 0.21 | 66.9 | 37.5 |
| E. coli | 105A | 1 | 0.00 | 0.48 | 1.00 | — | 0.0 | 100.0 |
| E. coli | 105A | 2 | 1.30 | 0.00 | 1.00 | — | 100.0 | 0.0 |
| E. coli | 105A | 2 | 0.87 | 0.24 | 1.17 | 0.28 | 66.9 | 50.0 |
| E. coli | 105A | 2 | 0.49 | 0.18 | 0.75 | 0.37 | 37.7 | 37.5 |
| E. coli | 105A | 2 | 0.87 | 0.18 | 1.04 | 0.21 | 66.9 | 37.5 |
| E. coli | 105A | 2 | 0.00 | 0.48 | 1.00 | — | 0.0 | 100.0 |
| E. coli | 213A | 1 | 1.30 | 0.00 | 1.00 | — | 100.0 | 0.0 |
| E. coli | 213A | 1 | 0.65 | 0.18 | 1.00 | 0.28 | 50.0 | 50.0 |
| E. coli | 213A | 1 | 0.65 | 0.24 | 1.17 | 0.37 | 50.0 | 66.7 |
| E. coli | 213A | 1 | 0.87 | 0.18 | 1.17 | 0.21 | 66.9 | 50.0 |
| E. coli | 213A | 1 | 0.00 | 0.36 | 1.00 | — | 0.0 | 100.0 |
| E. coli | 213A | 2 | 1.30 | 0.00 | 1.00 | — | 100.0 | 0.0 |
| E. coli | 213A | 2 | 0.87 | 0.24 | 1.34 | 0.28 | 66.9 | 66.7 |
| E. coli | 213A | 2 | 0.65 | 0.24 | 1.17 | 0.37 | 50.0 | 66.7 |
| E. coli | 213A | 2 | 0.00 | 0.36 | 1.00 | — | 0.0 | 100.0 |
| E. coli | 223A | 1 | 1.47 | 0.00 | 1.00 | — | 100.0 | 0.0 |
| E. coli | 223A | 1 | 0.73 | 0.22 | 1.16 | 0.30 | 49.7 | 66.7 |
| E. coli | 223A | 1 | 0.55 | 0.22 | 1.04 | 0.39 | 37.4 | 66.7 |
| E. coli | 223A | 1 | 0.73 | 0.16 | 1.00 | 0.22 | 49.7 | 50.0 |
| E. coli | 223A | 1 | 0.00 | 0.33 | 1.00 | — | 0.0 | 100.0 |
| E. coli | 223A | 2 | 1.47 | 0.00 | 1.00 | — | 100.0 | 0.0 |
| E. coli | 223A | 2 | 0.73 | 0.22 | 1.00 | 0.30 | 49.7 | 50.0 |
| E. coli | 223A | 2 | 0.73 | 0.16 | 0.87 | 0.22 | 49.7 | 37.5 |
| E. coli | 223A | 2 | 0.00 | 0.43 | 1.00 | — | 0.0 | 100.0 |

TABLE 12
Synergy testing of combination of Phenoxyethanol
(PE) and CHDM with P. aeruginosa

| Organism | Plate # | Day | Q_{A/a} | Q_{B/b} | SI | B/A | % CHDM | % PE |
|---|---|---|---|---|---|---|---|---|
| P. aeruginosa | 126E | 2 | 1.75 | 0.00 | 1.00 | — | 100.0 | 0.0 |
| P. aeruginosa | 126E | 2 | 0.88 | 0.21 | 1.17 | 0.23 | 50.3 | 66.7 |
| P. aeruginosa | 126E | 2 | 0.66 | 0.21 | 1.04 | 0.31 | 37.7 | 66.7 |
| P. aeruginosa | 126E | 2 | 1.17 | 0.21 | 1.34 | 0.18 | 66.9 | 66.7 |
| P. aeruginosa | 126E | 2 | 0.00 | 0.31 | 1.00 | — | 0.0 | 100.0 |

TABLE 13
Synergy testing of combination of Phenoxyethanol
(PE) and CHDM with S. aureus

| Organism | Plate # | Day | Q_{A/a} | Q_{B/b} | SI | B/A | % CHDM | % PE |
|---|---|---|---|---|---|---|---|---|
| S. aureus | 124D | 1 | 3.11 | 0.00 | 1.00 | — | 100.0 | 0.0 |
| S. aureus | 124D | 1 | 1.56 | 0.28 | 0.88 | 0.18 | 50.2 | 37.6 |
| S. aureus | 124D | 1 | 1.56 | 0.37 | 1.00 | 0.24 | 50.2 | 49.9 |
| S. aureus | 124D | 1 | 2.07 | 0.28 | 1.04 | 0.13 | 66.6 | 37.5 |
| S. aureus | 124D | 1 | 0.00 | 0.73 | 1.00 | — | 0.0 | 100.0 |
| S. aureus | 124D | 2 | 3.11 | 0.00 | 1.00 | — | 100.0 | 0.0 |
| S. aureus | 124D | 2 | 2.07 | 0.37 | 1.17 | 0.18 | 66.6 | 50.4 |
| S. aureus | 124D | 2 | 1.56 | 0.37 | 1.00 | 0.24 | 50.2 | 50.4 |
| S. aureus | 124D | 2 | 2.07 | 0.28 | 1.04 | 0.13 | 66.6 | 37.6 |
| S. aureus | 124D | 2 | 0.00 | 0.73 | 1.00 | — | 0.0 | 100.0 |
| S. aureus | 219D | 1 | 3.30 | 0.00 | 1.00 | — | 100.0 | 0.0 |
| S. aureus | 219D | 1 | 1.24 | 0.21 | 0.88 | 0.17 | 37.6 | 49.9 |
| S. aureus | 219D | 1 | 2.20 | 0.28 | 1.33 | 0.13 | 66.7 | 66.7 |
| S. aureus | 219D | 1 | 0.00 | 0.41 | 1.00 | — | 0.0 | 100.0 |
| S. aureus | 219D | 2 | 3.30 | 0.00 | 1.00 | — | 100.0 | 0.0 |
| S. aureus | 219D | 2 | 2.20 | 0.37 | 1.33 | 0.17 | 66.7 | 67.3 |
| S. aureus | 219D | 2 | 2.20 | 0.28 | 1.17 | 0.13 | 66.7 | 50.9 |
| S. aureus | 219D | 2 | 0.00 | 0.55 | 1.00 | — | 0.0 | 100.0 |
| S. aureus | 234D | 1 | 3.75 | 0.00 | 1.00 | — | 100.0 | 0.0 |
| S. aureus | 234D | 1 | 2.50 | 0.50 | 1.17 | 0.20 | 66.7 | 50.0 |
| S. aureus | 234D | 1 | 1.88 | 0.50 | 1.00 | 0.27 | 50.0 | 50.0 |
| S. aureus | 234D | 1 | 2.50 | 0.38 | 1.04 | 0.15 | 66.7 | 37.5 |
| S. aureus | 234D | 1 | 0.00 | 1.00 | 1.00 | — | 0.0 | 100.0 |

TABLE 14
Synergy testing of combination of Caprylyl glycol (CG) and CHDM with A. niger

| Organism | Plate # | Day | Q_{A/a} | Q_{B/b} | SI | B/A | % CHDM | % CG |
|---|---|---|---|---|---|---|---|---|
| A. niger | 123C | 2 | 3.11 | 0.00 | 1.00 | — | 100.0 | 0.0 |
| A. niger | 123C | 2 | 0.87 | 0.03 | 0.56 | 0.03 | 28.0 | 28.1 |
| A. niger | 123C | 2 | 1.56 | 0.07 | 1.17 | 0.04 | 50.2 | 66.7 |
| A. niger | 123C | 2 | 1.56 | 0.04 | 0.88 | 0.02 | 50.2 | 37.5 |
| A. niger | 123C | 2 | 0.00 | 0.10 | 1.00 | — | 0.0 | 100.0 |
| A. niger | 123C | 3 | 3.11 | 0.00 | 1.00 | — | 100.0 | 0.0 |
| A. niger | 123C | 3 | 2.07 | 0.07 | 1.17 | 0.03 | 66.6 | 50.0 |
| A. niger | 123C | 3 | 1.56 | 0.07 | 1.00 | 0.04 | 50.2 | 50.0 |
| A. niger | 123C | 3 | 2.07 | 0.05 | 1.04 | 0.02 | 66.6 | 37.5 |
| A. niger | 123C | 3 | 0.00 | 0.13 | 1.00 | — | 0.0 | 100.0 |
| A. niger | 217C | 2 | 4.50 | 0.00 | 1.00 | — | 100.0 | 0.0 |
| A. niger | 217C | 2 | 3.00 | 0.09 | 1.17 | 0.03 | 66.7 | 50.0 |
| A. niger | 217C | 2 | 2.25 | 0.09 | 1.00 | 0.04 | 50.0 | 50.0 |
| A. niger | 217C | 2 | 3.00 | 0.07 | 1.04 | 0.02 | 66.7 | 37.5 |
| A. niger | 217C | 2 | 0.00 | 0.17 | 1.00 | — | 0.0 | 100.0 |

TABLE 15
Synergy testing of combination of Caprylyl
glycol (CG) and CHDM with C. albicans

| Organism | Plate # | Day | Q_{A/a} | Q_{B/b} | SI | B/A | % CHDM | % CG |
|---|---|---|---|---|---|---|---|---|
| C. albicans | 117B | 2 | 3.56 | 0.00 | 1.00 | — | 100.0 | 0.0 |
| C. albicans | 117B | 2 | 1.34 | 0.04 | 0.75 | 0.03 | 37.6 | 37.5 |
| C. albicans | 117B | 2 | 1.34 | 0.06 | 0.88 | 0.04 | 37.6 | 50.0 |
| C. albicans | 117B | 2 | 1.34 | 0.03 | 0.66 | 0.02 | 37.6 | 28.1 |
| C. albicans | 117B | 2 | 0.00 | 0.11 | 1.00 | — | 0.0 | 100.0 |
| C. albicans | 117B | 3 | 4.75 | 0.00 | 1.00 | — | 100.0 | 0.0 |
| C. albicans | 117B | 3 | 1.78 | 0.06 | 0.88 | 0.03 | 37.5 | 50.0 |
| C. albicans | 117B | 3 | 1.78 | 0.08 | 1.04 | 0.04 | 37.5 | 66.7 |
| C. albicans | 117B | 3 | 2.38 | 0.06 | 1.00 | 0.02 | 50.1 | 50.0 |
| C. albicans | 117B | 3 | 0.00 | 0.11 | 1.00 | — | 0.0 | 100.0 |
| C. albicans | 226B | 2 | 5.63 | 0 | 1.00 | — | 100.0 | 0.0 |
| C. albicans | 226B | 2 | 3.75 | 0.12 | 1.17 | 0.03 | 66.6 | 50.0 |
| C. albicans | 226B | 2 | 2.81 | 0.12 | 1.00 | 0.04 | 49.9 | 50.0 |
| C. albicans | 226B | 2 | 3.75 | 0.09 | 1.04 | 0.02 | 66.6 | 37.5 |
| C. albicans | 226B | 2 | 0 | 0.24 | 1.00 | — | 0.0 | 100.0 |
| C. albicans | 254B | 2 | 5.00 | 0.00 | 1.00 | — | 100.0 | 0.0 |
| C. albicans | 254B | 2 | 2.50 | 0.11 | 1.17 | 0.04 | 50.0 | 66.7 |
| C. albicans | 254B | 2 | 2.50 | 0.15 | 1.39 | 0.06 | 50.0 | 88.5 |
| C. albicans | 254B | 2 | 3.33 | 0.11 | 1.33 | 0.03 | 66.6 | 66.7 |
| C. albicans | 254B | 2 | 0.00 | 0.17 | 1.00 | — | 0.0 | 100.0 |
| C. albicans | 254B | 3 | 6.67 | 0.00 | 1.00 | — | 100.0 | 0.0 |
| C. albicans | 254B | 3 | 3.33 | 0.15 | 1.17 | 0.04 | 49.9 | 66.7 |
| C. albicans | 254B | 3 | 2.50 | 0.15 | 1.04 | 0.06 | 37.5 | 66.7 |
| C. albicans | 254B | 3 | 3.33 | 0.11 | 1.00 | 0.03 | 49.9 | 50.0 |
| C. albicans | 254B | 3 | 0.00 | 0.22 | 1.00 | — | 0.0 | 100.0 |

TABLE 16
Synergy testing of combination of Caprylyl glycol (CG) and CHDM with E. coli

| Organism | Plate # | Day | Q_{A/a} | Q_{B/b} | SI | B/A | % CHDM | % CG |
|---|---|---|---|---|---|---|---|---|
| E. coli | 107A | 1 | 1.73 | 0.00 | 1.00 | — | 100.0 | 0.0 |
| E. coli | 107A | 1 | 0.87 | 0.08 | 1.00 | 0.09 | 50.3 | 50.0 |
| E. coli | 107A | 1 | 0.00 | 0.16 | 1.00 | — | 0.0 | 100.0 |
| E. coli | 107A | 2 | 1.73 | 0.00 | 1.00 | — | 100.0 | 0.0 |
| E. coli | 107A | 2 | 0.87 | 0.08 | 1.00 | 0.09 | 50.3 | 50.0 |
| E. coli | 107A | 2 | 0.00 | 0.16 | 1.00 | — | 0.0 | 100.0 |

TABLE 17
Synergy testing of combination of Caprylyl
glycol (CG) and CHDM with P. aeruginosa

| Organism | Plate # | Day | Q_{A/a} | Q_{B/b} | SI | B/A | % CHDM | % CG |
|---|---|---|---|---|---|---|---|---|
| P. aeruginosa | 169E | 1 | 1.31 | 0.00 | 1.00 | — | 100.0 | 0.0 |
| P. aeruginosa | 169E | 1 | 0.66 | 0.11 | 1.00 | 0.17 | 50.0 | 50.0 |
| P. aeruginosa | 169E | 1 | 0.66 | 0.15 | 1.17 | 0.23 | 50.0 | 66.7 |
| P. aeruginosa | 169E | 1 | 0.88 | 0.11 | 1.17 | 0.13 | 66.6 | 50.0 |
| P. aeruginosa | 169E | 1 | 0.00 | 0.23 | 1.00 | — | 0.0 | 100.0 |
| P. aeruginosa | 169E | 2 | 1.75 | 0.00 | 1.00 | — | 100.0 | 0.0 |
| P. aeruginosa | 169E | 2 | 1.17 | 0.20 | 1.34 | 0.17 | 66.7 | 66.7 |
| P. aeruginosa | 169E | 2 | 1.17 | 0.15 | 1.17 | 0.13 | 66.7 | 50.0 |
| P. aeruginosa | 169E | 2 | 0.00 | 0.30 | 1.00 | — | 0.0 | 100.0 |
| P. aeruginosa | 169E | 3 | 1.75 | 0.00 | 1.00 | — | 100.0 | 0.0 |
| P. aeruginosa | 169E | 3 | 1.17 | 0.20 | 1.34 | 0.17 | 66.7 | 66.7 |
| P. aeruginosa | 169E | 3 | 0.00 | 0.30 | 1.00 | — | 0.0 | 100.0 |

TABLE 18
Synergy testing of combination of Caprylyl
glycol (CG) and CHDM with S. aureus

| Organism | Plate # | Day | Q_{A/a} | Q_{B/b} | SI | B/A | % CHDM | % CG |
|---|---|---|---|---|---|---|---|---|
| S. aureus | 125D | 1 | 3.11 | 0.00 | 1.00 | — | 100.0 | 0.0 |
| S. aureus | 125D | 1 | 1.56 | 0.12 | 1.00 | 0.08 | 50.2 | 50.0 |
| S. aureus | 125D | 1 | 1.56 | 0.16 | 1.17 | 0.10 | 50.2 | 66.7 |
| S. aureus | 125D | 1 | 2.07 | 0.12 | 1.17 | 0.06 | 66.6 | 50.0 |
| S. aureus | 125D | 1 | 0.00 | 0.24 | 1.00 | — | 0.0 | 100.0 |
| S. aureus | 125D | 2 | 3.11 | 0.00 | 1.00 | — | 100.0 | 0.0 |
| S. aureus | 125D | 2 | 2.07 | 0.16 | 1.17 | 0.08 | 66.6 | 50.0 |
| S. aureus | 125D | 2 | 2.07 | 0.12 | 1.04 | 0.06 | 66.6 | 37.5 |
| S. aureus | 125D | 2 | 0.00 | 0.32 | 1.00 | — | 0.0 | 100.0 |

TABLE 19
Synergy testing of combination of Methylparaben (MP) and CHDM with A. niger

| Organism | Plate # | Day | Q_{A/a} | Q_{B/b} | SI | B/A | % CHDM | % MP |
|---|---|---|---|---|---|---|---|---|
| A. niger | 154C | 3 | 2.03 | 0.000 | 1.00 | — | 100.0 | 0.0 |
| A. niger | 154C | 3 | 1.35 | 0.025 | 1.17 | 0.02 | 66.5 | 50.0 |
| A. niger | 154C | 3 | 1.35 | 0.033 | 1.33 | 0.02 | 66.5 | 66.6 |
| A. niger | 154C | 3 | 0.00 | 0.050 | 1.00 | — | 0.0 | 100.0 |

TABLE 20
Synergy testing of combination of Methylparaben
(MP) and CHDM with C. albicans

| Organism | Plate # | Day | Q_{A/a} | Q_{B/b} | SI | B/A | % CHDM | % MP |
|---|---|---|---|---|---|---|---|---|
| C. albicans | 128B | 2 | 3.00 | 0.000 | 1.00 | — | 100.0 | 0.0 |
| C. albicans | 128B | 2 | 2.00 | 0.030 | 1.17 | 0.02 | 66.7 | 50.0 |
| C. albicans | 128B | 2 | 1.12 | 0.023 | 0.75 | 0.02 | 37.3 | 37.5 |
| C. albicans | 128B | 2 | 2.00 | 0.023 | 1.04 | 0.01 | 66.7 | 37.5 |
| C. albicans | 128B | 2 | 0.00 | 0.060 | 1.00 | — | 0.0 | 100.0 |
| C. albicans | 128B | 3 | 4.00 | 0.000 | 1.00 | — | 100.0 | 0.0 |
| C. albicans | 128B | 3 | 2.67 | 0.040 | 1.17 | 0.01 | 66.8 | 50.0 |
| C. albicans | 128B | 3 | 2.00 | 0.040 | 1.00 | 0.02 | 50.0 | 50.0 |
| C. albicans | 128B | 3 | 2.67 | 0.030 | 1.04 | 0.01 | 66.8 | 37.5 |
| C. albicans | 128B | 3 | 0.00 | 0.080 | 1.00 | — | 0.0 | 100.0 |

TABLE 21
Synergy testing of combination of Methylparaben (MP) and CHDM with E. coli

| Organism | Plate # | Day | Q_{A/a} | Q_{B/b} | SI | B/A | % CHDM | % MP |
|---|---|---|---|---|---|---|---|---|
| E. coli | 108A | 1 | 1.73 | 0.000 | 1.00 | — | 100.0 | 0.0 |
| E. coli | 108A | 1 | 0.87 | 0.075 | 1.17 | 0.09 | 50.3 | 66.7 |
| E. coli | 108A | 1 | 0.87 | 0.056 | 1.00 | 0.06 | 50.3 | 50.0 |
| E. coli | 108A | 1 | 0.00 | 0.112 | 1.00 | — | 0.0 | 100.0 |
| E. coli | 108A | 2 | 1.73 | 0.00 | 1.00 | — | 100.0 | 0.0 |
| E. coli | 108A | 2 | 0.87 | 0.075 | 1.17 | 0.09 | 50.3 | 66.7 |
| E. coli | 108A | 2 | 0.00 | 0.112 | 1.00 | — | 0.0 | 100.0 |

TABLE 22
Synergy testing of combination of Methylparaben
(MP) and CHDM with P. aeruginosa

| Organism | Plate # | Day | Q_{A/a} | Q_{B/b} | SI | B/A | % CHDM | % MP |
|---|---|---|---|---|---|---|---|---|
| P. aeruginosa | 146E | 1 | 1.31 | 0.00 | 1.00 | — | 100.0 | 0.0 |
| P. aeruginosa | 146E | 1 | 1.17 | 0.09 | 1.56 | 0.08 | 89.3 | 66.6 |
| P. aeruginosa | 146E | 1 | 0.88 | 0.09 | 1.34 | 0.11 | 67.2 | 66.6 |
| P. aeruginosa | 146E | 1 | 1.17 | 0.07 | 1.39 | 0.06 | 89.3 | 50.0 |
| P. aeruginosa | 146E | 1 | 0.00 | 0.14 | 1.00 | — | 0.0 | 100.0 |

TABLE 23
Synergy testing of combination of Methylparaben
(MP) and CHDM with S. aureus

| Organism | Plate # | Day | Q_{A/a} | Q_{B/b} | SI | B/A | % CHDM | % MP |
|---|---|---|---|---|---|---|---|---|
| S. aureus | 166D | 1 | 3.30 | 0.00 | 1.00 | — | 100.0 | 0.0 |
| S. aureus | 166D | 1 | 1.65 | 0.15 | 1.17 | 0.09 | 50.0 | 66.7 |
| S. aureus | 166D | 1 | 1.24 | 0.15 | 1.04 | 0.09 | 37.6 | 66.7 |
| S. aureus | 166D | 1 | 2.20 | 0.15 | 1.33 | 0.07 | 66.7 | 66.7 |
| S. aureus | 166D | 1 | 0.00 | 0.23 | 1.00 | — | 0.0 | 100.0 |
| S. aureus | 166D | 2 | 3.30 | 0.00 | 1.00 | — | 100.0 | 0.0 |
| S. aureus | 166D | 2 | 1.65 | 0.15 | 1.17 | 0.09 | 50.0 | 66.7 |
| S. aureus | 166D | 2 | 1.65 | 0.20 | 1.39 | 0.12 | 50.0 | 88.9 |
| S. aureus | 166D | 2 | 2.20 | 0.15 | 1.33 | 0.07 | 66.7 | 66.7 |
| S. aureus | 166D | 2 | 0.00 | 0.23 | 1.00 | — | 0.0 | 100.0 |

TABLE 24
Synergy testing of combination of Methylisothiazolinone
(MIT) and CHDM with A. niger

| Organism | Plate # | Day | Q_{A/a} | Q_{B/b} | SI | B/A | % CHDM | % MIT |
|---|---|---|---|---|---|---|---|---|
| A. niger | 193C | 2 | 4.93 | 0.00000 | 1.00 | — | 100.0 | 0.0 |
| A. niger | 193C | 2 | 2.47 | 0.02670 | 1.17 | 0.01081 | 50.1 | 66.8 |
| A. niger | 193C | 2 | 1.39 | 0.02000 | 0.78 | 0.01439 | 28.2 | 50.0 |
| A. niger | 193C | 2 | 2.47 | 0.02000 | 1.00 | 0.00810 | 50.1 | 50.0 |
| A. niger | 193C | 2 | 0.00 | 0.04000 | 1.00 | — | 0.0 | 100.0 |
| A. niger | 193C | 3 | 2.78 | 0.00000 | 1.00 | — | 100.0 | 0.0 |
| A. niger | 193C | 3 | 1.39 | 0.01500 | 1.00 | 0.01079 | 50.0 | 50.0 |
| A. niger | 193C | 3 | 1.39 | 0.02000 | 1.17 | 0.01439 | 50.0 | 66.7 |
| A. niger | 193C | 3 | 1.85 | 0.01500 | 1.17 | 0.00811 | 66.5 | 50.0 |
| A. niger | 193C | 3 | 0.00 | 0.03000 | 1.00 | — | 0.0 | 100.0 |
| A. niger | 214C | 2 | 4.50 | 0.00000 | 1.00 | — | 100.0 | 0.0 |
| A. niger | 214C | 2 | 3.00 | 0.02000 | 1.33 | 0.00667 | 66.7 | 50.0 |
| A. niger | 214C | 2 | 2.25 | 0.02670 | 1.17 | 0.01187 | 50.0 | 66.8 |
| A. niger | 214C | 2 | 3.00 | 0.02670 | 1.17 | 0.00890 | 66.7 | 66.8 |
| A. niger | 214C | 2 | 0.00 | 0.04000 | 1.00 | — | 0.0 | 100.0 |
| A. niger | 214C | 3 | 6.00 | 0.00000 | 1.00 | — | 100.0 | 0.0 |
| A. niger | 214C | 3 | 3.00 | 0.02670 | 1.17 | 0.00890 | 50.0 | 66.8 |
| A. niger | 214C | 3 | 3.00 | 0.02000 | 1.00 | 0.00667 | 50.0 | 50.0 |
| A. niger | 214C | 3 | 0.00 | 0.04000 | 1.00 | — | 0.0 | 100.0 |

TABLE 25
Synergy testing of combination of Methylisothiazolinone
(MIT) and CHDM with C. albicans

| Organism | Plate # | Day | Q_{A/a} | Q_{B/b} | SI | B/A | % CHDM | % MIT |
|---|---|---|---|---|---|---|---|---|
| C. albicans | 162B | 3 | 5.33 | 0.00000 | 1.00 | — | 100.0 | 0.0 |
| C. albicans | 162B | 3 | 2.67 | 0.00800 | 1.17 | 0.00300 | 50.1 | 66.7 |
| C. albicans | 162B | 3 | 2.00 | 0.008 | 1.04 | 0.00400 | 37.5 | 66.7 |
| C. albicans | 162B | 3 | 0.00 | 0.012 | 1.00 | — | 0.0 | 100.0 |
| C. albicans | 200B | 2 | 6.23 | 0.000 | 1.00 | — | 100.0 | 0.0 |
| C. albicans | 200B | 2 | 4.15 | 0.011 | 1.17 | 0.00258 | 66.6 | 50.2 |
| C. albicans | 200B | 2 | 3.11 | 0.011 | 1.00 | 0.00344 | 49.9 | 50.2 |
| C. albicans | 200B | 2 | 4.15 | 0.008 | 1.04 | 0.00193 | 66.6 | 37.6 |
| C. albicans | 200B | 2 | 0.00 | 0.021 | 1.00 | — | 0.0 | 100.0 |
| C. albicans | 200B | 3 | 6.23 | 0.000 | 1.00 | — | 100.0 | 0.0 |
| C. albicans | 200B | 3 | 3.11 | 0.008 | 0.87 | 0.00257 | 49.9 | 37.6 |
| C. albicans | 200B | 3 | 3.11 | 0.011 | 1.00 | 0.00344 | 49.9 | 50.2 |
| C. albicans | 200B | 3 | 4.15 | 0.008 | 1.04 | 0.00193 | 66.6 | 37.6 |
| C. albicans | 200B | 3 | 0.00 | 0.021 | 1.00 | — | 0.0 | 100.0 |

TABLE 26
Synergy testing of combination of Methylisothiazolinone
(MIT) and CHDM with P. aeruginosa

| Organism | Plate # | Day | Q_{A/a} | Q_{B/b} | SI | B/A | % CHDM | % MIT |
|---|---|---|---|---|---|---|---|---|
| P. aeruginosa | 147E | 1 | 1.31 | 0.00000 | 1.00 | — | 100.0 | 0.0 |
| P. aeruginosa | 147E | 1 | 0.66 | 0.00026 | 0.99 | 0.00039 | 50.4 | 49.1 |
| P. aeruginosa | 147E | 1 | 0.49 | 0.00026 | 0.86 | 0.00053 | 37.4 | 49.1 |
| P. aeruginosa | 147E | 1 | 0.88 | 0.00026 | 1.16 | 0.00030 | 67.2 | 49.1 |
| P. aeruginosa | 147E | 1 | 0.00 | 0.00053 | 1.00 | — | 0.0 | 100.0 |
| P. aeruginosa | 147E | 2 | 2.33 | 0.00000 | 1.00 | — | 100.0 | 0.0 |
| P. aeruginosa | 147E | 2 | 0.88 | 0.00035 | 0.75 | 0.00040 | 37.8 | 37.6 |
| P. aeruginosa | 147E | 2 | 0.66 | 0.00035 | 0.66 | 0.00053 | 28.3 | 37.6 |
| P. aeruginosa | 147E | 2 | 1.17 | 0.00035 | 0.88 | 0.00030 | 50.2 | 37.6 |
| P. aeruginosa | 147E | 2 | 0.00 | 0.00093 | 1.00 | — | 0.0 | 100.0 |
| P. aeruginosa | 210E | 1 | 1.75 | 0.00000 | 1.00 | — | 100.0 | 0.0 |
| P. aeruginosa | 210E | 1 | 0.66 | 0.00030 | 0.88 | 0.00045 | 37.7 | 50.0 |
| P. aeruginosa | 210E | 1 | 0.49 | 0.00030 | 0.78 | 0.00061 | 28.0 | 50.0 |
| P. aeruginosa | 210E | 1 | 0.88 | 0.00030 | 1.00 | 0.00034 | 50.3 | 50.0 |
| P. aeruginosa | 210E | 1 | 0.00 | 0.00060 | 1.00 | — | 0.0 | 100.0 |
| P. aeruginosa | 210E | 2 | 1.75 | 0.00000 | 1.00 | — | 100.0 | 0.0 |
| P. aeruginosa | 210E | 2 | 0.88 | 0.00040 | 0.90 | 0.00045 | 50.3 | 40.0 |
| P. aeruginosa | 210E | 2 | 0.66 | 0.00040 | 0.78 | 0.00061 | 37.7 | 40.0 |
| P. aeruginosa | 210E | 2 | 1.17 | 0.00040 | 1.07 | 0.00034 | 66.9 | 40.0 |
| P. aeruginosa | 210E | 2 | 0.00 | 0.00100 | 1.00 | — | 0.0 | 100.0 |
| P. aeruginosa | 249E | 1 | 1.80 | 0.00000 | 1.00 | — | 100.0 | 0.0 |
| P. aeruginosa | 249E | 1 | 0.68 | 0.00032 | 1.04 | 0.00047 | 37.8 | 66.7 |
| P. aeruginosa | 249E | 1 | 0.51 | 0.00032 | 0.95 | 0.00063 | 28.3 | 66.7 |
| P. aeruginosa | 249E | 1 | 0.90 | 0.00032 | 1.17 | 0.00036 | 50.0 | 66.7 |
| P. aeruginosa | 249E | 1 | 0.00 | 0.00048 | 1.00 | — | 0.0 | 100.0 |
| P. aeruginosa | 249E | 2 | 1.80 | 0.00000 | 1.00 | — | 100.0 | 0.0 |
| P. aeruginosa | 249E | 2 | 0.90 | 0.00040 | 0.94 | 0.00044 | 50.0 | 44.4 |
| P. aeruginosa | 249E | 2 | 0.68 | 0.00040 | 0.82 | 0.00059 | 37.8 | 44.4 |
| P. aeruginosa | 249E | 2 | 0.90 | 0.00030 | 0.83 | 0.00033 | 50.0 | 33.3 |
| P. aeruginosa | 249E | 2 | 0.00 | 0.00090 | 1.00 | — | 0.0 | 100.0 |

TABLE 27
Synergy testing of combination of Methylisothiazolinone
(MIT) and CHDM with S. aureus

| Organism | Plate # | Day | Q_{A/a} | Q_{B/b} | SI | B/A | % CHDM | % MIT |
|---|---|---|---|---|---|---|---|---|
| S. aureus | 137D | 2 | 4.15 | 0.0000 | 1.00 | — | 100.0 | 0.0 |
| S. aureus | 137D | 2 | 2.07 | 0.0013 | 1.15 | 0.00063 | 49.9 | 65.0 |
| S. aureus | 137D | 2 | 1.56 | 0.0013 | 1.03 | 0.00083 | 37.6 | 65.0 |
| S. aureus | 137D | 2 | 2.07 | 0.0010 | 1.00 | 0.00048 | 49.9 | 50.0 |
| S. aureus | 137D | 2 | 0 | 0.002 | 1.00 | — | 0.0 | 100.0 |

TABLE 28
Synergy testing of combination of Chlorphenesin (CP) and CHDM with A. niger

| Organism | Plate # | Day | Q_{A/a} | Q_{B/b} | SI | B/A | % CHDM | % CP |
|---|---|---|---|---|---|---|---|---|
| A. niger | 194C | 2 | 3.70 | 0.00 | 1.00 | — | 100.0 | 0.0 |
| A. niger | 194C | 2 | 2.47 | 0.11 | 1.33 | 0.044 | 66.8 | 66.6 |
| A. niger | 194C | 2 | 1.85 | 0.11 | 1.17 | 0.059 | 50.0 | 66.6 |
| A. niger | 194C | 2 | 2.47 | 0.09 | 1.17 | 0.033 | 66.8 | 50.0 |
| A. niger | 194C | 2 | 0.00 | 0.17 | 1.00 | — | 0.0 | 100.0 |
| A. niger | 194C | 3 | 2.78 | 0.00 | 1.00 | — | 100.0 | 0.0 |
| A. niger | 194C | 3 | 1.85 | 0.09 | 1.17 | 0.046 | 66.5 | 50.0 |
| A. niger | 194C | 3 | 1.39 | 0.09 | 1.00 | 0.061 | 50.0 | 50.0 |
| A. niger | 194C | 3 | 1.85 | 0.06 | 1.04 | 0.034 | 66.5 | 37.5 |
| A. niger | 194C | 3 | 0.00 | 0.17 | 1.00 | — | 0.0 | 100.0 |

TABLE 29
Synergy testing of combination of Chlorphenesin
(CP) and CHDM with C. albicans

| Organism | Plate # | Day | Q_{A/a} | Q_{B/b} | SI | B/A | % CHDM | % CP |
|---|---|---|---|---|---|---|---|---|
| C. albicans | 130B | 2 | 3.00 | 0 | 1.00 | — | 100.0 | 0.0 |
| C. albicans | 130B | 2 | 1.12 | 0.0712 | 0.75 | 0.0636 | 37.3 | 37.5 |
| C. albicans | 130B | 2 | 1.12 | 0.0949 | 0.87 | 0.0847 | 37.3 | 50.0 |
| C. albicans | 130B | 2 | 2.00 | 0.0949 | 1.17 | 0.0475 | 66.7 | 50.0 |
| C. albicans | 130B | 2 | 0 | 0.1898 | 1.00 | — | 0.0 | 100.0 |
| C. albicans | 130B | 3 | 4.00 | 0 | 1.00 | — | 100.0 | 0.0 |
| C. albicans | 130B | 3 | 2.00 | 0.1265 | 1.00 | 0.063 | 50.0 | 50.0 |
| C. albicans | 130B | 3 | 1.50 | 0.1265 | 0.88 | 0.084 | 37.5 | 50.0 |
| C. albicans | 130B | 3 | 2.67 | 0.1265 | 1.17 | 0.047 | 66.8 | 50.0 |
| C. albicans | 130B | 3 | 0 | 0.253 | 1.00 | — | 0.0 | 100.0 |
| C. albicans | 227B | 2 | 5.63 | 0 | 1.00 | — | 100.0 | 0.0 |
| C. albicans | 227B | 2 | 3.75 | 0.165 | 1.17 | 0.04 | 66.6 | 50.0 |
| C. albicans | 227B | 2 | 2.81 | 0.165 | 1.00 | 0.06 | 49.9 | 50.0 |
| C. albicans | 227B | 2 | 3.75 | 0.124 | 1.04 | 0.03 | 66.6 | 37.5 |
| C. albicans | 227B | 2 | 0 | 0.33 | 1.00 | — | 0.0 | 100.0 |
| C. albicans | 227B | 3 | 5.63 | 0.00 | 1.00 | — | 100.0 | 0.0 |
| C. albicans | 227B | 3 | 3.75 | 0.17 | 1.17 | 0.04 | 66.6 | 50.0 |
| C. albicans | 227B | 3 | 3.75 | 0.22 | 1.33 | 0.06 | 66.6 | 66.7 |
| C. albicans | 227B | 3 | 5.00 | 0.17 | 1.39 | 0.03 | 88.8 | 50.0 |
| C. albicans | 227B | 3 | 0.00 | 0.33 | 1.00 | — | 0.0 | 100.0 |
| C. albicans | 256B | 2 | 6.67 | 0.00 | 1.00 | — | 100.0 | 0.0 |
| C. albicans | 256B | 2 | 3.33 | 0.20 | 1.17 | 0.06 | 49.9 | 66.7 |
| C. albicans | 256B | 2 | 2.50 | 0.20 | 1.04 | 0.08 | 37.5 | 66.7 |
| C. albicans | 256B | 2 | 3.33 | 0.15 | 1.00 | 0.05 | 49.9 | 50.0 |
| C. albicans | 256B | 2 | 0.00 | 0.30 | 1.00 | — | 0.0 | 100.0 |
| C. albicans | 256B | 3 | 6.67 | 0.00 | 1.00 | — | 100.0 | 0.0 |
| C. albicans | 256B | 3 | 3.33 | 0.20 | 1.17 | 0.06 | 49.9 | 66.7 |
| C. albicans | 256B | 3 | 0.0 | 0.30 | 1.00 | — | 0.0 | 100.0 |

TABLE 30
Synergy testing of combination of Chlorphenesin (CP) and CHDM with E. coli

| Organism | Plate # | Day | Q_{A/a} | Q_{B/b} | SI | B/A | % CHDM | % CP |
|---|---|---|---|---|---|---|---|---|
| E. coli | 110A | 1 | 1.73 | 0.00 | 1.00 | — | 100.0 | 0.0 |
| E. coli | 110A | 1 | 0.87 | 0.11 | 1.20 | 0.123 | 50.3 | 66.7 |
| E. coli | 110A | 1 | 0.87 | 0.08 | 1.00 | 0.092 | 50.3 | 50.0 |
| E. coli | 110A | 1 | 0.00 | 0.16 | 1.00 | — | 0.0 | 100.0 |
| E. coli | 110A | 2 | 1.73 | 0.00 | 1.00 | — | 100.0 | 0.0 |
| E. coli | 110A | 2 | 0.87 | 0.11 | 1.20 | 0.123 | 50.3 | 66.7 |
| E. coli | 110A | 2 | 0.87 | 0.08 | 1.00 | 0.092 | 50.3 | 50.0 |
| E. coli | 110A | 2 | 0.00 | 0.16 | 1.00 | — | 0.0 | 100.0 |

TABLE 31
Synergy testing of combination of Chlorphenesin
(CP) and CHDM with P. aeruginosa

| Organism | Plate # | Day | Q_{A/a} | Q_{B/b} | SI | B/A | % CHDM | % CP |
|---|---|---|---|---|---|---|---|---|
| P. aeruginosa | 148E | 1 | 1.31 | 0.00 | 1.00 | — | 100.0 | 0.0 |
| P. aeruginosa | 148E | 1 | 1.17 | 0.21 | 1.56 | 0.18 | 89.3 | 66.7 |
| P. aeruginosa | 148E | 1 | 0.88 | 0.21 | 1.34 | 0.24 | 67.2 | 66.7 |
| P. aeruginosa | 148E | 1 | 1.17 | 0.16 | 1.39 | 0.13 | 89.3 | 50.0 |
| P. aeruginosa | 148E | 1 | 0.00 | 0.31 | 1.00 | — | 0.0 | 100.0 |

TABLE 32
Synergy testing of combination of Chlorphenesin (CP) and CHDM with S. aureus

| Organism | Plate # | Day | Q_{A/a} | Q_{B/b} | SI | B/A | % CHDM | % CP |
|---|---|---|---|---|---|---|---|---|
| S. aureus | 138D | 1 | 3.11 | 0.00 | 1.00 | — | 100.0 | 0.0 |
| S. aureus | 138D | 1 | 2.07 | 0.20 | 1.17 | 0.10 | 66.6 | 50.0 |
| S. aureus | 138D | 1 | 1.56 | 0.20 | 1.00 | 0.13 | 50.2 | 50.0 |
| S. aureus | 138D | 1 | 2.07 | 0.15 | 1.04 | 0.07 | 66.6 | 37.5 |
| S. aureus | 138D | 1 | 0.00 | 0.40 | 1.00 | — | 0.0 | 100.0 |
| S. aureus | 138D | 2 | 4.15 | 0.00 | 1.00 | — | 100.0 | 0.0 |
| S. aureus | 138D | 2 | 2.07 | 0.20 | 1.00 | 0.097 | 49.9 | 50.0 |
| S. aureus | 138D | 2 | 0.00 | 0.40 | 1.00 | — | 0.0 | 100.0 |

TABLE 33
Synergy testing of combination of BA:DHA and CHDM with A. niger

| Organism | Plate # | Day | Q_{A/a} | Q_{B/b} | SI | B/A | % CHDM | % BA:DHA |
|---|---|---|---|---|---|---|---|---|
| A. niger | 195C | 2 | 4.93 | 0.000 | 1.00 | — | 100.0 | 0.0 |
| A. niger | 195C | 2 | 2.47 | 0.070 | 1.17 | 0.03 | 50.1 | 66.7 |
| A. niger | 195C | 2 | 1.85 | 0.070 | 1.04 | 0.04 | 37.5 | 66.7 |
| A. niger | 195C | 2 | 2.47 | 0.052 | 1.00 | 0.02 | 50.1 | 50.0 |
| A. niger | 195C | 2 | 0.00 | 0.104 | 1.00 | — | 0.0 | 100.0 |
| A. niger | 195C | 3 | 4.93 | 0.000 | 1.00 | — | 100.0 | 0.0 |
| A. niger | 195C | 3 | 2.47 | 0.070 | 1.00 | 0.03 | 50.1 | 50.2 |
| A. niger | 195C | 3 | 1.85 | 0.070 | 0.88 | 0.04 | 37.5 | 50.2 |
| A. niger | 195C | 3 | 2.47 | 0.052 | 0.88 | 0.02 | 50.1 | 37.6 |
| A. niger | 195C | 3 | 0.00 | 0..139 | 1.00 | — | 0.0 | 100.0 |
| A. niger | 215C | 2 | 4.50 | 0.000 | 1.00 | — | 100.0 | 0.0 |
| A. niger | 215C | 2 | 2.25 | 0.052 | 1.00 | 0.02 | 50.0 | 50.0 |
| A. niger | 215C | 2 | 2.25 | 0.070 | 1.17 | 0.03 | 50.0 | 66.7 |
| A. niger | 215C | 2 | 3.00 | 0.052 | 1.17 | 0.02 | 66.7 | 50.0 |
| A. niger | 215C | 2 | 0.00 | 0.104 | 1.00 | — | 0.0 | 100.0 |
| A. niger | 215C | 3 | 4.50 | 0.000 | 1.00 | — | 100.0 | 0.0 |
| A. niger | 215C | 3 | 2.25 | 0.052 | 0.88 | 0.02 | 50.0 | 37.6 |
| A. niger | 215C | 3 | 2.25 | 0.070 | 1.00 | 0.03 | 50.0 | 50.2 |
| A. niger | 215C | 3 | 3.00 | 0.052 | 1.04 | 0.02 | 66.7 | 37.6 |
| A. niger | 215C | 3 | 0.00 | 0.139 | 1.00 | — | 0.0 | 100.0 |

TABLE 34
Synergy testing of combination of BA:DHA and CHDM with C. albicans

| Organism | Plate # | Day | Q_{A/a} | Q_{B/b} | SI | B/A | % CHDM | % BA:DHA |
|---|---|---|---|---|---|---|---|---|
| C. albicans | 131B | 3 | 5.33 | 0.000 | 1.00 | — | 100.0 | 0.0 |
| C. albicans | 131B | 3 | 2.00 | 0.065 | 1.04 | 0.03 | 37.5 | 66.7 |
| C. albicans | 131B | 3 | 1.50 | 0.065 | 0.95 | 0.04 | 28.1 | 66.7 |
| C. albicans | 131B | 3 | 2.67 | 0.065 | 1.17 | 0.02 | 50.1 | 66.7 |
| C. albicans | 131B | 3 | 0 | 0.097 | 1.00 | — | 0.0 | 100.0 |
| C. albicans | 131B | 4 | 5.33 | 0.000 | 1.00 | — | 100.0 | 0.0 |
| C. albicans | 131B | 4 | 2.00 | 0.065 | 0.88 | 0.03 | 37.5 | 50.0 |
| C. albicans | 131B | 4 | 1.50 | 0.065 | 0.78 | 0.04 | 28.1 | 50.0 |
| C. albicans | 131B | 4 | 2.67 | 0.065 | 1.00 | 0.02 | 50.1 | 50.0 |
| C. albicans | 131B | 4 | 0 | 0.129 | 1.00 | — | 0.0 | 100.0 |
| C. albicans | 239B | 2 | 5.85 | 0.000 | 1.00 | — | 100.0 | 0.0 |
| C. albicans | 239B | 2 | 3.90 | 0.089 | 1.33 | 0.02 | 66.7 | 66.7 |
| C. albicans | 239B | 2 | 2.93 | 0.089 | 1.17 | 0.03 | 50.1 | 66.7 |
| C. albicans | 239B | 2 | 3.90 | 0.067 | 1.17 | 0.02 | 66.7 | 50.0 |
| C. albicans | 239B | 2 | 0 | 0.133 | 1.00 | — | 0.0 | 100.0 |
| C. albicans | 239B | 3 | 5.85 | 0.000 | 1.00 | — | 100.0 | 0.0 |
| C. albicans | 239B | 3 | 3.90 | 0.089 | 1.17 | 0.02 | 66.7 | 50.0 |
| C. albicans | 239B | 3 | 3.90 | 0.118 | 1.33 | 0.03 | 66.7 | 66.6 |
| C. albicans | 239B | 3 | 5.20 | 0.089 | 1.39 | 0.02 | 88.9 | 50.0 |
| C. albicans | 239B | 3 | 0 | 0.177 | 1.00 | — | 0.0 | 100.0 |
| C. albicans | 258B | 2 | 5.00 | 0.000 | 1.00 | — | 100.0 | 0.0 |
| C. albicans | 258B | 2 | 2.50 | 0.078 | 1.00 | 0.03 | 50.0 | 50.0 |
| C. albicans | 258B | 2 | 1.88 | 0.078 | 0.88 | 0.04 | 37.6 | 50.0 |
| C. albicans | 258B | 2 | 3.33 | 0.078 | 1.17 | 0.02 | 66.7 | 50.0 |
| C. albicans | 258B | 2 | 0.0 | 0.156 | 1.00 | — | 0.0 | 100.0 |
| C. albicans | 258B | 3 | 6.67 | 0.000 | 1.00 | — | 100.0 | 0.0 |
| C. albicans | 258B | 3 | 3.33 | 0.104 | 1.17 | 0.03 | 49.9 | 66.7 |
| C. albicans | 258B | 3 | 0.0 | 0.156 | 1.00 | — | 0.0 | 100.0 |

TABLE 35
Synergy testing of combination of BA:DHA and CHDM with E. coli

| Organism | Plate # | Day | Q_{A/a} | Q_{B/b} | SI | B/A | % CHDM | % BA:DHA |
|---|---|---|---|---|---|---|---|---|
| E. coli | 111A | 1 | 1.73 | 0.000 | 1.00 | — | 100.0 | 0.0 |
| E. coli | 111A | 1 | 0.87 | 0.273 | 1.00 | 0.301 | 50.3 | 50.0 |
| E. coli | 111A | 1 | 0.00 | 0.546 | 1.00 | — | 0.0 | 100.0 |

TABLE 36
Synergy testing of combination of BA:DHA and CHDM with P. aeruginosa

| Organism | Plate # | Day | Q_{A/a} | Q_{B/b} | SI | B/A | % CHDM | % BA:DHA |
|---|---|---|---|---|---|---|---|---|
| P. aeruginosa | 149E | 1 | 1.31 | 0.000 | 1.00 | — | 100.0 | 0.0 |
| P. aeruginosa | 149E | 1 | 0.88 | 0.115 | 1.17 | 0.13 | 67.2 | 50.0 |
| P. aeruginosa | 149E | 1 | 0.66 | 0.115 | 1.00 | 0.17 | 50.4 | 50.0 |
| P. aeruginosa | 149E | 1 | 1.17 | 0.115 | 1.39 | 0.10 | 89.3 | 50.0 |
| P. aeruginosa | 149E | 1 | 0.00 | 0.229 | 1.00 | — | 0.0 | 100.0 |
| P. aeruginosa | 149E | 2 | 1.75 | 0.000 | 1.00 | — | 100.0 | 0.0 |
| P. aeruginosa | 149E | 2 | 1.17 | 0.153 | 1.17 | 0.131 | 66.9 | 50.1 |
| P. aeruginosa | 149E | 2 | 0.00 | 0.306 | 1.00 | — | 0.0 | 100.0 |

TABLE 37
Synergy testing of combination of BA:DHA and CHDM with S. aureus

| Organism | Plate # | Day | Q_{A/a} | Q_{B/b} | SI | B/A | % CHDM | % BA:DHA |
|---|---|---|---|---|---|---|---|---|
| S. aureus | 139D | 1 | 3.11 | 0.000 | 1.00 | — | 100.0 | 0.0 |
| S. aureus | 139D | 1 | 1.17 | 0.196 | 0.88 | 0.16 | 37.6 | 50.0 |
| S. aureus | 139D | 1 | 0.87 | 0.196 | 0.78 | 0.22 | 28.0 | 50.0 |
| S. aureus | 139D | 1 | 1.56 | 0.196 | 1.00 | 0.12 | 50.2 | 50.0 |
| S. aureus | 139D | 1 | 0 | 0.391 | 1.00 | — | 0.0 | 100.0 |
| S. aureus | 139D | 2 | 4.15 | 0.000 | 1.00 | — | 100.0 | 0.0 |
| S. aureus | 139D | 2 | 2.07 | 0.348 | 1.17 | 0.16 | 49.9 | 66.7 |
| S. aureus | 139D | 2 | 1.56 | 0.348 | 1.04 | 0.21 | 37.6 | 66.7 |
| S. aureus | 139D | 2 | 2.07 | 0.261 | 1.00 | 0.12 | 49.9 | 50.0 |
| S. aureus | 139D | 2 | 0 | 0.521 | 1.00 | — | 0.0 | 100.0 |
| S. aureus | 220D | 1 | 3.30 | 0.000 | 1.00 | — | 100.0 | 0.0 |
| S. aureus | 220D | 1 | 1.24 | 0.184 | 0.88 | 0.14 | 37.6 | 49.9 |
| S. aureus | 220D | 1 | 0.93 | 0.184 | 0.78 | 0.19 | 28.2 | 50.0 |
| S. aureus | 220D | 1 | 1.65 | 0.184 | 1.00 | 0.11 | 50.0 | 50.0 |
| S. aureus | 220D | 1 | 0 | 0.368 | 1.00 | — | 0.0 | 100.0 |
| S. aureus | 220D | 2 | 3.30 | 0.000 | 1.00 | — | 100.0 | 0.0 |
| S. aureus | 220D | 2 | 2.20 | 0.327 | 1.33 | 0.14 | 66.7 | 66.7 |
| S. aureus | 220D | 2 | 1.65 | 0.327 | 1.17 | 0.19 | 50.0 | 66.7 |
| S. aureus | 220D | 2 | 2.20 | 0.245 | 1.17 | 0.11 | 66.7 | 50.0 |
| S. aureus | 220D | 2 | 0 | 0.490 | 1.00 | — | 0.0 | 100.0 |
| S. aureus | 235D | 1 | 5.00 | 0.000 | 1.00 | — | 100.0 | 0.0 |
| S. aureus | 235D | 1 | 2.50 | 0.591 | 1.17 | 0.24 | 50.0 | 66.7 |
| S. aureus | 235D | 1 | 1.88 | 0.591 | 1.04 | 0.31 | 37.6 | 66.7 |
| S. aureus | 235D | 1 | 2.50 | 0.443 | 1.00 | 0.18 | 50.0 | 50.0 |
| S. aureus | 235D | 1 | 0 | 0.886 | 1.00 | — | 0.0 | 100.0 |
| S. aureus | 235D | 2 | 5.00 | 0.000 | 1.00 | — | 100.0 | 0.0 |
| S. aureus | 235D | 2 | 2.50 | 0.591 | 1.17 | 0.24 | 50.0 | 66.7 |
| S. aureus | 235D | 2 | 1.88 | 0.591 | 1.04 | 0.31 | 37.6 | 66.7 |
| S. aureus | 235D | 2 | 2.50 | 0.443 | 1.00 | 0.18 | 50.0 | 50.0 |
| S. aureus | 235D | 2 | 0 | 0.886 | 1.00 | — | 0.0 | 100.0 |

TABLE 38
Synergy testing of combination of DMDM hydantoin and CHDM with A. niger

| Organism | Plate # | Day | Q_{A/a} | Q_{B/b} | SI | B/A | % CHDM | % DMDMH |
|---|---|---|---|---|---|---|---|---|
| A. niger | 196C | 3 | 2.78 | 0.00 | 1.00 | — | 100.0 | 0.0 |
| A. niger | 196C | 3 | 2.47 | 0.09 | 1.55 | 0.038 | 88.8 | 66.6 |
| A. niger | 196C | 3 | 2.47 | 0.07 | 1.39 | 0.028 | 88.8 | 50.0 |
| A. niger | 196C | 3 | 0.00 | 0.14 | 1.00 | — | 0.0 | 100.0 |

TABLE 39
Synergy testing of combination of DMDM
hydantoin and CHDM with C. albicans

| Organism | Plate # | Day | Q_{A/a} | Q_{B/b} | SI | B/A | % CHDM | % DMDMH |
|---|---|---|---|---|---|---|---|---|
| C. albicans | 241B | 2 | 5.85 | 0.00 | 1.00 | — | 100.0 | 0.0 |
| C. albicans | 241B | 2 | 2.93 | 0.06 | 0.78 | 0.02 | 50.1 | 28.1 |
| C. albicans | 241B | 2 | 2.19 | 0.06 | 0.66 | 0.03 | 37.4 | 28.1 |
| C. albicans | 241B | 2 | 2.93 | 0.05 | 0.71 | 0.02 | 50.1 | 21.1 |
| C. albicans | 241B | 2 | 0.00 | 0.22 | 1.00 | — | 0.0 | 100.0 |
| C. albicans | 241B | 3 | 5.85 | 0.00 | 1.00 | — | 100.0 | 0.0 |
| C. albicans | 241B | 3 | 2.93 | 0.062 | 0.78 | 0.02 | 50.0 | 28.1 |
| C. albicans | 241B | 3 | 2.93 | 0.083 | 0.88 | 0.03 | 50.0 | 37.5 |
| C. albicans | 241B | 3 | 2.93 | 0.046 | 0.71 | 0.02 | 50.0 | 21.1 |
| C. albicans | 241B | 3 | 0.00 | 0.22 | 1.00 | — | 0.0 | 100.0 |
| C. albicans | 244B | 2 | 6.00 | 0.00 | 1.00 | — | 100.0 | 0.0 |
| C. albicans | 244B | 2 | 3.00 | 0.14 | 0.88 | 0.05 | 50.0 | 37.5 |
| C. albicans | 244B | 2 | 2.25 | 0.14 | 0.75 | 0.06 | 37.5 | 37.5 |
| C. albicans | 244B | 2 | 3.00 | 0.10 | 0.78 | 0.03 | 50.0 | 28.1 |
| C. albicans | 244B | 2 | 0.00 | 0.36 | 1.00 | — | 0.0 | 100.0 |
| C. albicans | 244B | 3 | 8.00 | 0.00 | 1.00 | — | 100.0 | 0.0 |
| C. albicans | 244B | 3 | 3.00 | 0.14 | 0.75 | 0.05 | 37.5 | 37.5 |
| C. albicans | 244B | 3 | 3.00 | 0.18 | 0.88 | 0.06 | 37.5 | 50.0 |
| C. albicans | 244B | 3 | 4.00 | 0.14 | 0.88 | 0.03 | 50.0 | 37.5 |
| C. albicans | 244B | 3 | 0.00 | 0.36 | 1.00 | — | 0.0 | 100.0 |
| C. albicans | 263B | 2 | 8.00 | 0.00 | 1.00 | — | 100.0 | 0.0 |
| C. albicans | 263B | 2 | 4.00 | 0.40 | 1.39 | 0.10 | 50.0 | 88.9 |
| C. albicans | 263B | 2 | 1.69 | 0.23 | 0.71 | 0.13 | 21.1 | 50.0 |
| C. albicans | 263B | 2 | 2.25 | 0.17 | 0.66 | 0.07 | 28.1 | 37.5 |
| C. albicans | 263B | 2 | 0.00 | 0.45 | 1.00 | — | 0.0 | 100.0 |
| C. albicans | 263B | 3 | 8.00 | 0.00 | 1.00 | — | 100.0 | 0.0 |
| C. albicans | 263B | 3 | 4.00 | 0.40 | 1.00 | 0.10 | 50.0 | 50.0 |
| C. albicans | 263B | 3 | 2.25 | 0.30 | 0.66 | 0.13 | 28.1 | 37.5 |
| C. albicans | 263B | 3 | 3.00 | 0.23 | 0.66 | 0.08 | 37.5 | 28.1 |
| C. albicans | 263B | 3 | 0.00 | 0.80 | 1.00 | — | 0.0 | 100.0 |
| C. albicans | 264B | 2 | 6.67 | 0.00 | 1.00 | — | 100.0 | 0.0 |
| C. albicans | 264B | 2 | 1.88 | 0.17 | 0.78 | 0.09 | 28.2 | 50.0 |
| C. albicans | 264B | 2 | 1.41 | 0.17 | 0.71 | 0.12 | 21.1 | 50.0 |
| C. albicans | 264B | 2 | 1.88 | 0.13 | 0.66 | 0.07 | 28.2 | 37.5 |
| C. albicans | 264B | 2 | 0.00 | 0.34 | 1.00 | — | 0.0 | 100.0 |
| C. albicans | 264B | 3 | 6.67 | 0.00 | 1.00 | — | 100.0 | 0.0 |
| C. albicans | 264B | 3 | 2.50 | 0.23 | 0.75 | 0.09 | 37.5 | 37.5 |
| C. albicans | 264B | 3 | 2.50 | 0.30 | 0.87 | 0.12 | 37.5 | 50.0 |
| C. albicans | 264B | 3 | 2.50 | 0.17 | 0.66 | 0.07 | 37.5 | 28.0 |
| C. albicans | 264B | 3 | 0.00 | 0.60 | 1.00 | — | 0.0 | 100.0 |

TABLE 40
Synergy testing of combination of DMDM hydantoin
and CHDM with P. aeruginosa

| Organism | Plate # | Day | Q_{A/a} | Q_{B/b} | SI | B/A | % CHDM | % DMDMH |
|---|---|---|---|---|---|---|---|---|
| P. aeruginosa | 150E | 1 | 1.31 | 0.000 | 1.00 | — | 100.0 | 0.0 |
| P. aeruginosa | 150E | 1 | 1.17 | 0.025 | 1.39 | 0.021 | 89.3 | 50.1 |
| P. aeruginosa | 150E | 1 | 0.00 | 0.049 | 1.00 | — | 0.0 | 100.0 |

TABLE 41
Synergy testing of combination of Iodopropynyl
butylcarbamate (IPBC) and CHDM with A. niger

| Organism | Plate # | Day | Q_{A/a} | Q_{B/b} | SI | B/A | % CHDM | % IPBC |
|---|---|---|---|---|---|---|---|---|
| A. niger | 259C | 2 | 5.00 | 0.000000 | 1.00 | — | 100.0 | 0.0 |
| A. niger | 259C | 2 | 3.33 | 0.000020 | 1.17 | 0.00 | 66.6 | 50.0 |
| A. niger | 259C | 2 | 3.33 | 0.000015 | 1.04 | 0.00 | 66.6 | 37.5 |
| A. niger | 259C | 2 | 0.00 | 0.000040 | 1.00 | — | 0.0 | 100.0 |
| A. niger | 260C | 3 | 6.50 | 0.000000 | 1.00 | — | 100.0 | 0.0 |
| A. niger | 260C | 3 | 4.33 | 0.000040 | 1.56 | 0.00 | 66.6 | 88.9 |
| A. niger | 260C | 3 | 3.25 | 0.000040 | 1.39 | 0.00 | 50.0 | 88.9 |
| A. niger | 260C | 3 | 4.33 | 0.000030 | 1.33 | 0.00 | 66.6 | 66.7 |
| A. niger | 260C | 3 | 0.00 | 0.000045 | 1.00 | — | 0.0 | 100.0 |

TABLE 42
Synergy testing of combination of Iodopropynyl
butylcarbamate (IPBC) and CHDM with E. coli

| Organism | Plate # | Day | Q_{A/a} | Q_{B/b} | SI | B/A | % CHDM | % IPBC |
|---|---|---|---|---|---|---|---|---|
| E. coli | 119A | 1 | 1.73 | 0.000 | 1.00 | — | 100.0 | 0.0 |
| E. coli | 119A | 1 | 1.15 | 0.005 | 1.56 | 0.004 | 66.5 | 89.1 |
| E. coli | 119A | 1 | 1.15 | 0.004 | 1.34 | 0.003 | 66.5 | 67.3 |
| E. coli | 119A | 1 | 0.00 | 0.006 | 1.00 | — | 0.0 | 100.0 |
| E. coli | 119A | 2 | 1.73 | 0.000 | 1.00 | — | 100.0 | 0.0 |
| E. coli | 119A | 2 | 1.15 | 0.005 | 1.17 | 0.004 | 66.5 | 50.5 |
| E. coli | 119A | 2 | 1.15 | 0.004 | 1.05 | 0.003 | 66.5 | 38.1 |
| E. coli | 119A | 2 | 0.00 | 0.010 | 1.00 | — | 0.0 | 100.0 |

TABLE 43
Synergy testing of combination of Iodopropynyl butylcarbamate
(IPBC) and CHDM with P. aeruginosa

| Organism | Plate # | Day | Q_{A/a} | Q_{B/b} | SI | B/A | % CHDM | % IPBC |
|---|---|---|---|---|---|---|---|---|
| P. aeruginosa | 151E | 1 | 1.31 | 0.000 | 1.00 | — | 100.0 | 0.0 |
| P. aeruginosa | 151E | 1 | 0.66 | 0.024 | 0.79 | 0.04 | 50.4 | 28.1 |
| P. aeruginosa | 151E | 1 | 0.66 | 0.033 | 0.88 | 0.05 | 50.4 | 37.5 |
| P. aeruginosa | 151E | 1 | 0.66 | 0.018 | 0.71 | 0.03 | 50.4 | 21.1 |
| P. aeruginosa | 151E | 1 | 0.00 | 0.087 | 1.00 | — | 0.0 | 100.0 |
| P. aeruginosa | 151E | 2 | 1.75 | 0.000 | 1.00 | — | 100.0 | 0.0 |
| P. aeruginosa | 151E | 2 | 0.88 | 0.033 | 0.88 | 0.04 | 50.3 | 37.5 |
| P. aeruginosa | 151E | 2 | 0.88 | 0.043 | 1.00 | 0.05 | 50.3 | 49.9 |
| P. aeruginosa | 151E | 2 | 1.17 | 0.033 | 1.04 | 0.03 | 66.9 | 37.5 |
| P. aeruginosa | 151E | 2 | 0.00 | 0.087 | 1.00 | — | 0.0 | 100.0 |
| P. aeruginosa | 170E | 2 | 1.75 | 0.000 | 1.00 | — | 100.0 | 0.0 |
| P. aeruginosa | 170E | 2 | 0.88 | 0.055 | 0.88 | 0.06 | 50.3 | 37.5 |
| P. aeruginosa | 170E | 2 | 0.88 | 0.041 | 0.78 | 0.05 | 50.3 | 28.2 |
| P. aeruginosa | 170E | 2 | 0.00 | 0.147 | 1.00 | — | 0.0 | 100.0 |
| P. aeruginosa | 187E | 1 | 1.31 | 0.000 | 1.00 | — | 100.0 | 0.0 |
| P. aeruginosa | 187E | 1 | 0.49 | 0.025 | 0.66 | 0.05 | 37.4 | 28.1 |
| P. aeruginosa | 187E | 1 | 0.37 | 0.025 | 0.56 | 0.07 | 28.2 | 28.1 |
| P. aeruginosa | 187E | 1 | 0.49 | 0.025 | 0.66 | 0.05 | 37.4 | 28.1 |
| P. aeruginosa | 187E | 1 | 0.00 | 0.090 | 1.00 | — | 0.0 | 100.0 |
| P. aeruginosa | 187E | 2 | 1.75 | 0.000 | 1.00 | — | 100.0 | 0.0 |
| P. aeruginosa | 187E | 2 | 0.49 | 0.025 | 0.49 | 0.05 | 28.0 | 21.1 |
| P. aeruginosa | 187E | 2 | 0.66 | 0.045 | 0.75 | 0.07 | 37.7 | 37.5 |
| P. aeruginosa | 187E | 2 | 0.88 | 0.034 | 0.78 | 0.04 | 50.3 | 28.2 |
| P. aeruginosa | 187E | 2 | 0.00 | 0.120 | 1.00 | — | 0.0 | 100.0 |
| P. aeruginosa | 188E | 2 | 1.75 | 0.000 | 1.00 | — | 100.0 | 0.0 |
| P. aeruginosa | 188E | 2 | 0.66 | 0.064 | 0.66 | 0.10 | 37.7 | 28.1 |
| P. aeruginosa | 188E | 2 | 0.66 | 0.085 | 0.75 | 0.13 | 37.7 | 37.5 |
| P. aeruginosa | 188E | 2 | 0.66 | 0.048 | 0.59 | 0.07 | 37.7 | 21.1 |
| P. aeruginosa | 188E | 2 | 0.00 | 0.227 | 1.00 | — | 0.0 | 100.0 |
| P. aeruginosa | 204E | 1 | 1.35 | 0.000 | 1.00 | — | 100.0 | 0.0 |
| P. aeruginosa | 204E | 1 | 0.38 | 0.023 | 0.66 | 0.06 | 28.1 | 37.5 |
| P. aeruginosa | 204E | 1 | 0.38 | 0.030 | 0.78 | 0.08 | 28.1 | 50.0 |
| P. aeruginosa | 204E | 1 | 0.38 | 0.017 | 0.56 | 0.04 | 28.1 | 28.2 |
| P. aeruginosa | 204E | 1 | 0.00 | 0.060 | 1.00 | — | 0.0 | 100.0 |
| P. aeruginosa | 204E | 2 | 1.80 | 0.000 | 1.00 | — | 100.0 | 0.0 |
| P. aeruginosa | 204E | 2 | 0.38 | 0.023 | 0.42 | 0.06 | 21.1 | 21.1 |
| P. aeruginosa | 204E | 2 | 0.51 | 0.040 | 0.66 | 0.08 | 28.1 | 37.5 |
| P. aeruginosa | 204E | 2 | 0.68 | 0.030 | 0.66 | 0.04 | 37.5 | 28.1 |
| P. aeruginosa | 204E | 2 | 0.00 | 0.107 | 1.00 | — | 0.0 | 100.0 |
| P. aeruginosa | 246E | 1 | 1.35 | 0.000 | 1.00 | — | 100.0 | 0.0 |
| P. aeruginosa | 246E | 1 | 0.38 | 0.035 | 0.49 | 0.09 | 28.1 | 21.1 |
| P. aeruginosa | 246E | 1 | 0.28 | 0.035 | 0.42 | 0.12 | 20.7 | 21.1 |
| P. aeruginosa | 246E | 1 | 0.38 | 0.026 | 0.44 | 0.07 | 28.1 | 15.8 |
| P. aeruginosa | 246E | 1 | 0.00 | 0.165 | 1.00 | — | 0.0 | 100.0 |
| P. aeruginosa | 246E | 2 | 1.35 | 0.000 | 1.00 | — | 100.0 | 0.0 |
| P. aeruginosa | 246E | 2 | 0.68 | 0.062 | 0.75 | 0.09 | 50.4 | 37.5 |
| P. aeruginosa | 246E | 2 | 0.68 | 0.046 | 0.66 | 0.07 | 50.4 | 28.1 |
| P. aeruginosa | 246E | 2 | 0.00 | 0.165 | 1.00 | — | 0.0 | 100.0 |

TABLE 44
Synergy testing of combination of Benzisothiazolinone
(BIT) and CHDM with E. coli

| Organism | Plate # | Day | Q_{A/a} | Q_{B/b} | SI | B/A | % CHDM | % BIT |
|---|---|---|---|---|---|---|---|---|
| E. coli | 120A | 1 | 1.73 | 0.0000 | 1.00 | — | 100.0 | 0.0 |
| E. coli | 120A | 1 | 1.15 | 0.00019 | 1.18 | 0.00017 | 66.5 | 51.4 |
| E. coli | 120A | 1 | 1.15 | 0.00014 | 1.04 | 0.00012 | 66.5 | 37.8 |
| E. coli | 120A | 1 | 0.00 | 0.00037 | 1.00 | — | 0.0 | 100.0 |
| E. coli | 120A | 2 | 1.73 | 0.0000 | 1.00 | — | 100.0 | 0.0 |
| E. coli | 120A | 2 | 1.15 | 0.00019 | 1.18 | 0.00017 | 66.5 | 51.4 |
| E. coli | 120A | 2 | 0.00 | 0.00037 | 1.00 | — | 0.0 | 100.0 |

TABLE 45
Synergy testing of combination of Benzisothiazolinone
(BIT) and CHDM with P. aeruginosa

| Organism | Plate # | Day | Q_{A/a} | Q_{B/b} | SI | B/A | % CHDM | % BIT |
|---|---|---|---|---|---|---|---|---|
| P. aeruginosa | 152E | 1 | 1.31 | 0.0000 | 1.00 | — | 100.0 | 0.0 |
| P. aeruginosa | 152E | 1 | 0.66 | 0.0008 | 0.79 | 0.0013 | 50.4 | 28.6 |
| P. aeruginosa | 152E | 1 | 0.66 | 0.0011 | 0.88 | 0.0017 | 50.4 | 37.9 |
| P. aeruginosa | 152E | 1 | 0.66 | 0.0006 | 0.72 | 0.0009 | 50.4 | 21.4 |
| P. aeruginosa | 152E | 1 | 0.00 | 0.0029 | 1.00 | — | 0.0 | 100.0 |
| P. aeruginosa | 152E | 2 | 2.33 | 0.0000 | 1.00 | — | 100.0 | 0.0 |
| P. aeruginosa | 152E | 2 | 0.88 | 0.0011 | 0.76 | 0.0013 | 37.8 | 37.9 |
| P. aeruginosa | 152E | 2 | 0.88 | 0.0015 | 0.89 | 0.0017 | 37.8 | 51.7 |
| P. aeruginosa | 152E | 2 | 1.17 | 0.0011 | 0.88 | 0.0009 | 50.2 | 37.9 |
| P. aeruginosa | 152E | 2 | 0.00 | 0.0029 | 1.00 | — | 0.0 | 100.0 |
| P. aeruginosa | 171E | 2 | 1.75 | 0.0000 | 1.00 | — | 100.0 | 0.0 |
| P. aeruginosa | 171E | 2 | 0.66 | 0.0013 | 0.67 | 0.0020 | 37.7 | 28.9 |
| P. aeruginosa | 171E | 2 | 0.66 | 0.0017 | 0.75 | 0.0026 | 37.7 | 37.8 |
| P. aeruginosa | 171E | 2 | 0.88 | 0.0013 | 0.79 | 0.0015 | 50.3 | 28.9 |
| P. aeruginosa | 171E | 2 | 0.00 | 0.0045 | 1.00 | — | 0.0 | 100.0 |
| P. aeruginosa | 211E | 1 | 1.31 | 0.0000 | 1.00 | — | 100.0 | 0.0 |
| P. aeruginosa | 211E | 1 | 0.49 | 0.0008 | 0.72 | 0.0016 | 37.4 | 34.8 |
| P. aeruginosa | 211E | 1 | 0.37 | 0.0008 | 0.63 | 0.0022 | 28.2 | 34.8 |
| P. aeruginosa | 211E | 1 | 0.49 | 0.0006 | 0.63 | 0.0012 | 37.4 | 26.1 |
| P. aeruginosa | 211E | 1 | 0.00 | 0.0023 | 1.00 | — | 0.0 | 100.0 |
| P. aeruginosa | 211E | 2 | 1.75 | 0.0000 | 1.00 | — | 100.0 | 0.0 |
| P. aeruginosa | 211E | 2 | 0.66 | 0.0011 | 0.65 | 0.0017 | 37.7 | 27.5 |
| P. aeruginosa | 211E | 2 | 0.66 | 0.0015 | 0.75 | 0.0023 | 37.7 | 37.5 |
| P. aeruginosa | 211E | 2 | 0.49 | 0.0006 | 0.43 | 0.0012 | 28.0 | 15.0 |
| P. aeruginosa | 211E | 2 | 0.00 | 0.0040 | 1.00 | — | 0.0 | 100.0 |
| P. aeruginosa | 237E | 1 | 1.35 | 0.0000 | 1.00 | — | 100.0 | 0.0 |
| P. aeruginosa | 237E | 1 | 0.38 | 0.0006 | 0.48 | 0.0016 | 28.1 | 19.4 |
| P. aeruginosa | 237E | 1 | 0.51 | 0.0012 | 0.76 | 0.0024 | 37.8 | 38.7 |
| P. aeruginosa | 237E | 1 | 0.51 | 0.0006 | 0.57 | 0.0012 | 37.8 | 19.4 |
| P. aeruginosa | 237E | 1 | 0.00 | 0.0031 | 1.00 | — | 0.0 | 100.0 |
| P. aeruginosa | 237E | 2 | 1.80 | 0.0000 | 1.00 | — | 100.0 | 0.0 |
| P. aeruginosa | 237E | 2 | 0.68 | 0.0012 | 0.76 | 0.0018 | 37.8 | 38.7 |
| P. aeruginosa | 237E | 2 | 0.68 | 0.0015 | 0.86 | 0.0022 | 37.8 | 48.4 |
| P. aeruginosa | 237E | 2 | 0.68 | 0.0009 | 0.67 | 0.0013 | 37.8 | 29.0 |
| P. aeruginosa | 237E | 2 | 0.00 | 0.0031 | 1.00 | — | 0.0 | 100.0 |
| P. aeruginosa | 261E | 1 | 1.80 | 0.0000 | 1.00 | — | 100.0 | 0.0 |
| P. aeruginosa | 261E | 1 | 1.20 | 0.0007 | 0.83 | 0.0006 | 66.7 | 16.7 |
| P. aeruginosa | 261E | 1 | 0.90 | 0.0007 | 0.67 | 0.0008 | 50.0 | 16.7 |
| P. aeruginosa | 261E | 1 | 1.20 | 0.0005 | 0.79 | 0.0004 | 66.7 | 11.9 |
| P. aeruginosa | 262E | 1 | 0.00 | 0.0042 | 1.00 | — | 0.0 | 100.0 |
| P. aeruginosa | 261E | 2 | 1.80 | 0.0000 | 1.00 | — | 100.0 | 0.0 |
| P. aeruginosa | 261E | 2 | 1.20 | 0.0007 | 0.83 | 0.0006 | 66.7 | 16.7 |
| P. aeruginosa | 262E | 2 | 0.00 | 0.0042 | 1.00 | — | 0.0 | 100.0 |
| P. aeruginosa | 261E | 1 | 1.80 | 0.0000 | 1.00 | — | 100.0 | 0.0 |
| P. aeruginosa | 262E | 1 | 0.60 | 0.0016 | 0.71 | 0.0027 | 33.3 | 38.1 |
| P. aeruginosa | 262E | 1 | 0.45 | 0.0016 | 0.63 | 0.0036 | 25.0 | 38.1 |
| P. aeruginosa | 262E | 1 | 0.80 | 0.0016 | 0.83 | 0.0020 | 44.4 | 38.1 |
| P. aeruginosa | 262E | 1 | 0.00 | 0.0042 | 1.00 | — | 0.0 | 100.0 |
| P. aeruginosa | 247E | 1 | 1.80 | 0.0000 | 1.00 | 0.0000 | 100.0 | 0.0 |
| P. aeruginosa | 248E | 1 | 0.38 | 0.0005 | 0.42 | 0.0013 | 21.0 | 20.9 |
| P. aeruginosa | 248E | 1 | 0.45 | 0.0006 | 0.50 | 0.0022 | 25.0 | 24.8 |
| P. aeruginosa | 248E | 1 | 0.51 | 0.0004 | 0.44 | 0.0007 | 28.1 | 16.1 |
| P. aeruginosa | 248E | 1 | 0.40 | 0.0007 | 0.52 | 0.0017 | 22.0 | 29.6 |
| P. aeruginosa | 247E | 1 | 0.38 | 0.0005 | 0.43 | 0.0013 | 21.1 | 21.7 |
| P. aeruginosa | 247E | 1 | 0.51 | 0.0009 | 0.67 | 0.0018 | 28.3 | 39.1 |
| P. aeruginosa | 247E | 1 | 0.51 | 0.0005 | 0.50 | 0.0010 | 28.3 | 21.7 |
| P. aeruginosa | 247E | 1 | 0.00 | 0.0023 | 1.00 | — | 0.0 | 100.0 |
| P. aeruginosa | 247E | 2 | 1.80 | 0.0000 | 1.00 | — | 100.0 | 0.0 |
| P. aeruginosa | 248E | 2 | 0.50 | 0.0006 | 0.55 | 0.0013 | 28.0 | 27.4 |
| P. aeruginosa | 247E | 2 | 0.90 | 0.0011 | 0.98 | 0.0012 | 50.0 | 47.8 |
| P. aeruginosa | 247E | 2 | 0.68 | 0.0011 | 0.86 | 0.0016 | 37.8 | 47.8 |
| P. aeruginosa | 247E | 2 | 0.90 | 0.0009 | 0.89 | 0.0010 | 50.0 | 39.1 |
| P. aeruginosa | 247E | 2 | 0.00 | 0.0023 | 1.00 | — | 0.0 | 100.0 |

TABLE 46
Synergy testing of combination of BIT:MIT and CHDM with C. albicans

| Organism | Plate # | Day | Q_{A/a} | Q_{B/b} | SI | B/A | % CHDM | % BIT/MIT |
|---|---|---|---|---|---|---|---|---|
| C. albicans | 176B | 2 | 3.38 | 0.0000 | 1.00 | — | 100.0 | 0.0 |
| C. albicans | 176B | 2 | 3.00 | 0.0015 | 1.57 | 0.00050 | 88.8 | 68.2 |
| C. albicans | 176B | 2 | 2.25 | 0.0015 | 1.35 | 0.00067 | 66.6 | 68.2 |
| C. albicans | 176B | 2 | 0.00 | 0.0022 | 1.00 | — | 0.0 | 100.0 |
| C. albicans | 176B | 3 | 4.50 | 0.0000 | 1.00 | — | 100.0 | 0.0 |
| C. albicans | 176B | 3 | 0.00 | 0.0029 | 1.00 | — | 0.0 | 100.0 |
| C. albicans | 135B | 2 | 4.00 | 0.0000 | 1.00 | — | 100.0 | 0.0 |
| C. albicans | 135B | 2 | 2.67 | 0.0010 | 1.17 | 0.00037 | 66.7 | 50.0 |
| C. albicans | 135B | 2 | 2.67 | 0.0008 | 1.04 | 0.00028 | 66.7 | 37.5 |
| C. albicans | 135B | 2 | 0.00 | 0.0020 | 1.00 | — | 0.0 | 100.0 |

TABLE 47
Synergy testing of combination of BIT:MIT and CHDM with E. coli

| Organism | Plate # | Day | Q_{A/a} | Q_{B/b} | SI | B/A | % CHDM | % BIT/MIT |
|---|---|---|---|---|---|---|---|---|
| E. coli | 121A | 1 | 1.73 | 0.00000 | 1.00 | — | 100.0 | 0.0 |
| E. coli | 121A | 1 | 1.15 | 0.00022 | 1.54 | 0.00019 | 66.5 | 88.0 |
| E. coli | 121A | 1 | 1.15 | 0.00017 | 1.34 | 0.00015 | 66.5 | 68.0 |
| E. coli | 121A | 1 | 0.00 | 0.00025 | 1.00 | — | 0.0 | 100.0 |
| E. coli | 121A | 2 | 1.73 | 0.00000 | 1.00 | — | 100.0 | 0.0 |
| E. coli | 121A | 2 | 1.15 | 0.00022 | 1.16 | 0.00019 | 66.5 | 50.0 |
| E. coli | 121A | 2 | 1.15 | 0.00017 | 1.05 | 0.00015 | 66.5 | 37.5 |
| E. coli | 121A | 2 | 0.00 | 0.00044 | 1.00 | — | 0.0 | 100.0 |
| E. coli | 115A | 1 | 1.73 | 0.00000 | 1.00 | — | 100.0 | 0.0 |
| E. coli | 115A | 1 | 0.87 | 0.00033 | 1.39 | 0.00038 | 50.0 | 88.8 |
| E. coli | 115A | 1 | 0.65 | 0.00033 | 1.26 | 0.00051 | 37.5 | 88.8 |
| E. coli | 115A | 1 | 0.00 | 0.00038 | 1.00 | — | 0.0 | 100.0 |
| E. coli | 115A | 2 | 1.73 | 0.00000 | 1.00 | — | 100.0 | 0.0 |
| E. coli | 115A | 2 | 0.87 | 0.00033 | 1.17 | 0.00038 | 50.0 | 66.6 |
| E. coli | 115A | 2 | 0.00 | 0.00050 | 1.00 | — | 0.0 | 100.0 |

TABLE 48
Synergy testing of combination of BIT:MIT and CHDM with P. aeruginosa

| Organism | Plate # | Day | QA/a | QB/b | SI | B/A | % CHDM | % BIT/MIT |
|---|---|---|---|---|---|---|---|---|
| P. aeruginosa | 153E | 1 | 1.31 | 0.00000 | 1.00 | — | 100.0 | 0.0 |
| P. aeruginosa | 153E | 1 | 0.66 | 0.00045 | 1.00 | 0.00068 | 50.4 | 50.0 |
| P. aeruginosa | 153E | 1 | 0.66 | 0.00060 | 1.17 | 0.00091 | 50.4 | 66.7 |
| P. aeruginosa | 153E | 1 | 0.66 | 0.00034 | 0.88 | 0.00052 | 50.4 | 37.8 |
| P. aeruginosa | 153E | 1 | 0.00 | 0.00090 | 1.00 | — | 0.0 | 100.0 |
| P. aeruginosa | 153E | 2 | 2.33 | 0.00000 | 1.00 | — | 100.0 | 0.0 |
| P. aeruginosa | 153E | 2 | 1.17 | 0.00060 | 0.88 | 0.00051 | 50.2 | 37.5 |
| P. aeruginosa | 153E | 2 | 0.66 | 0.00060 | 0.66 | 0.00091 | 28.3 | 37.5 |
| P. aeruginosa | 153E | 2 | 1.17 | 0.00060 | 0.88 | 0.00051 | 50.2 | 37.5 |
| P. aeruginosa | 153E | 2 | 0.00 | 0.00160 | 1.00 | — | 0.0 | 100.0 |
| P. aeruginosa | 172E | 2 | 1.75 | 0.00000 | 1.00 | — | 100.0 | 0.0 |
| P. aeruginosa | 172E | 2 | 0.66 | 0.00080 | 0.85 | 0.00121 | 37.7 | 47.1 |
| P. aeruginosa | 172E | 2 | 0.49 | 0.00080 | 0.75 | 0.00163 | 28.0 | 47.1 |
| P. aeruginosa | 172E | 2 | 0.66 | 0.00060 | 0.73 | 0.00091 | 37.7 | 35.3 |
| P. aeruginosa | 172E | 2 | 0.00 | 0.00170 | 1.00 | — | 0.0 | 100.0 |
| P. aeruginosa | 212E | 1 | 1.75 | 0.00000 | 1.00 | — | 100.0 | 0.0 |
| P. aeruginosa | 212E | 1 | 0.49 | 0.00050 | 0.57 | 0.00102 | 28.0 | 29.4 |
| P. aeruginosa | 212E | 1 | 0.49 | 0.00060 | 0.63 | 0.00122 | 28.0 | 35.3 |
| P. aeruginosa | 212E | 1 | 0.66 | 0.00050 | 0.67 | 0.00076 | 37.7 | 29.4 |
| P. aeruginosa | 212E | 1 | 0.00 | 0.00170 | 1.00 | — | 0.0 | 100.0 |
| P. aeruginosa | 212E | 2 | 1.75 | 0.00000 | 1.00 | — | 100.0 | 0.0 |
| P. aeruginosa | 212E | 2 | 0.66 | 0.00060 | 0.73 | 0.00091 | 37.7 | 35.3 |
| P. aeruginosa | 212E | 2 | 0.66 | 0.00090 | 0.91 | 0.00136 | 37.7 | 52.9 |
| P. aeruginosa | 212E | 2 | 0.88 | 0.00060 | 0.86 | 0.00068 | 50.3 | 35.3 |
| P. aeruginosa | 212E | 2 | 0.00 | 0.00170 | 1.00 | — | 0.0 | 100.0 |

TABLE 49
Synergy testing of combination of BIT:MIT and CHDM with S. aureus

| Organism | Plate # | Day | Q_{A/a} | Q_{B/b} | SI | B/A | % CHDM | % BIT/MIT |
|---|---|---|---|---|---|---|---|---|
| S. aureus | 143D | 1 | 3.11 | 0.00000 | 1.00 | — | 100.0 | 0.0 |
| S. aureus | 143D | 1 | 2.07 | 0.00020 | 1.54 | 0.00010 | 66.6 | 87.0 |
| S. aureus | 143D | 1 | 1.56 | 0.00020 | 1.37 | 0.00013 | 50.2 | 87.0 |
| S. aureus | 143D | 1 | 0.00 | 0.00023 | 1.00 | — | 0.0 | 100.0 |
| S. aureus | 143D | 2 | 4.15 | 0.00000 | 1.00 | — | 100.0 | 0.0 |
| S. aureus | 143D | 2 | 2.07 | 0.00020 | 1.17 | 0.00010 | 49.9 | 66.7 |
| S. aureus | 143D | 2 | 0.00 | 0.00030 | 1.00 | — | 0.0 | 100.0 |

### Example 3

### Examples 3.1-3.12

### Testing for Adequate Preservation of Mixtures in Cream Formulations

A test for adequate preservation was carried out in accordance with the European Pharmacopea (6.0) and United States Pharmacopea (5.1). The testing consisted of the inoculation of a skin cream formulation serving as an emulsion substrate. The skin cream formulation having a pH of 6.75 was as follows:

| | Wt % |
|---|---|
| Part A: Water Phase | |
| Deionized water | 88.1 |
| Glycerin | 2.0 |
| Carbopol Ultrez 10 Carbomer | 0.2 |
| Part B: Oil Phase | |
| Promulgen D Cetearyl Alcohol (and) | 2.0 |
| Ceteareth-20 | |
| Lexemul GDL Glyceryl Dilaurate | 0.5 |
| Cetyl Alcohol | 1.5 |
| Dow Corning 200 Fluid 350 cSt. | 0.2 |
| Dimethicone | |
| NutriLayer Oryza Sative (Rice) Bran Oil | 5.0 |
| Extract | |
| Part C: Neutralizer | |
| Triethanolamine, 50% in water | 0.5 |

This skin cream was the emulsion substrate, which formed the base for all further experimentation. Samples were prepared by adding the CHDM, preservative, and/or 1,2-octanediol at the concentration indicated in Table 50.

TABLE 50
Emulsion Substrate Additives

| Example | Description |
|---|---|
| 3.1 | Emulsion substrate (no additives) |
| 3.2 | Emulsion substrate with 0.75% CHDM-D90 |
| 3.3 | Emulsion substrate with 1.5% CHDM-D90 |
| 3.4 | Emulsion substrate with 2.5% CHDM-D90 |
| 3.5 | Emulsion substrate with 0.3% phenoxyethanol |
| 3.6 | Emulsion substrate with 0.3% phenoxyethanol + 1.5% |
| | CHDM-D90 |
| 3.7 | Emulsion substrate with 0.3% phenoxyethanol + 0.2% 1,2- |
| | octanediol |
| 3.8 | Emulsion substrate with 0.05% methylparaben |
| 3.9 | Emulsion substrate with 0.05% methylparaben + 1.5% |
| | CHDM-D90 |
| 3.10 | Emulsion substrate with 0.005% IPBC + 1.5% CHDM-D90 |
| 3.11 | Emulsion substrate with 0.3% 1,2-octanediol |
| 3.12 | Emulsion substrate with 0.15% 1,2-octanediol + |
| | 0.15% CHDM-D90 |

For Examples 3.1 through 3.10, 390.0 g cream were weighed into a 600-ml beaker. The cream was stirred at room temperature while adding the ingredients specified in Table 51. Each sample was stirred for 2 hours, then placed in the refrigerator until inoculated.

TABLE 51
Emulsion Substrate Additives

| Example | Ingredients Added to Skin Cream |
|---|---|
| 3.1 | Water (10.0 g) was added |
| 3.2 | CHDM-D90 (3.00 g) and 7.00 g water were added |
| 3.3 | CHDM-D90 (6.00 g) and 4.00 g water were added |
| 3.4 | CHDM-D90 (10.0 g) was added |
| 3.5 | Phenoxyethanol (1.20 g) and 8.80 g water were added |
| 3.6 | A premix was prepared by dissolving 2.40 g |
| | phenoxyethanol in 12.00 g CHDM-D90. Then, 7.20 g of |
| | the premix and 2.80 g water were added to the cream. |
| 3.7 | A premix was prepared by mixing 2.40 g phenoxyethanol |
| | and 1.60 g 1,2-octanediol. Then, 2.00 g of the premix and |
| | 8.00 g water were added to the cream. |
| 3.8 | Methylparaben (0.200 g) and 9.80 water were added. |
| 3.9 | A premix was prepared by dissolving 2.00 g |
| | methylparaben in 60.00 g CHDM-D90. Then, 6.20 g of the |
| | premix and 3.80 g water were added to the cream. |
| 3.10 | A premix was prepared by dissolving 0.200 g IPBC in |
| | 60.00 g CHDM-D90. Then, 6.02 g of this premix and 3.98 g |
| | water were added. |
| 3.11 | 1,2-Octanediol (0.552 g) and 4.05 g water were added. |
| 3.12 | A premix was prepared by dissolving 3.00 g 1,2-octanediol |
| | in 3.00 g CHDM-D90. Then, 0.552 g of the premix and |
| | 4.05 g water were added to the cream. |

For Examples 3.11 and 3.12, 179.4 g cream were weighed into a 400-ml beaker. The cream was stirred at room temperature while adding the specified ingredients.

The samples of Examples 3.1 through 3.10 were challenged with specific organisms (see Table 51) to produce a contamination of between 1.0×10⁵ cfu/g and 1.0×10⁶ cfu/g. The actual inoculation counts resulting from these challenges were immediately determined by diluting in sterile buffered water and (spread plate method) plating for enumeration. The results of these counts for the challenge organisms are shown in Table 52.

TABLE 52

| Challenge Organisms | cfu/g |
|---|---|
| A = Pseudomonas aeruginosa ATCC | 182,000 |
| 9027 | |
| B = Staphylococcus aureus ATCC 6538 | 184,000 |
| C = Candida albicans ATCC 10231 | 202,000 |
| D = Escherichia coli ATCC 8739 | 187,000 |
| E = Burkholderia cepacia | 179,000 |
| F = Aspergillus niger ATCC 16404 | 174,000 |

Challenge organisms were prepared in Mueller-Hinton broth, allowed to grow for 72 hours at 35° C.+/−2° C., centrifuged at 2500 rpm for 5 minutes, and the supernatant broth was removed. The microbial pellet was then re-diluted with sterile buffered water to a turbidity that matched previous 1.0×10⁸ cfu/g concentrations of that organism's specific growth curve.

Samples of Examples 3.11 and 3.12 were not challenged with Burkholderia cepacia due to limited test material. Otherwise, they were treated exactly the same as the test samples of Examples 3.1 through 3.10.

The test emulsions were maintained within a specific temperature range optimal for the organisms; 35° C.+/2° C. for the bacteria and 22° C.+/−2° C. for the fungi, for the first three days. They were kept at ambient room temperature for the subsequent time periods.

Subculture samples of approximately 1 gram were taken for counts at 7, 14, and 30 days and incubated under optimal conditions and nutrition for no less than 5 days. Subcultures were diluted 1:2, 1:10, 1:100, . . . , 1:10,000 and plated using the spread plate method onto Plate Count Agar and onto SAB Dextrose Agar for the Candida and Aspergillus species; and incubated as follows: 35° C.+/−2° C. for the Plate Count Agar and 22° C.+/−2° C. for the SAB Dextrose plates of Candida albicans and Aspergillus niger. Negative results were not reported before 7 days incubation, and counts were performed after no less than 5 days incubation. Because of the high viscosity of the test emulsion, at least a 1:2 dilution was required to perform the spread plate subcultures. 0-30 counts represent a 1 to 2 dilution, numbers 1-200 a 1:10 dilution; and the rest represent dilutions of 1:100, 1:1000, or 1:10,000. Counts of Candida and Aspergillus species were made on the agar representing the highest count observed, usually the SAB Dextrose.

Counts were adjusted in accordance to the weight of the subculture sample. Results are shown in Table 53.

TABLE 53
Microorganism Counts, cfu/g

| | Example | | | | | | |
|---|---|---|---|---|---|---|---|
| | 3.1 | 3.2 | 3.3 | 3.4 | 3.5 | 3.6 | 3.7 |
| | Antimicrobial System | | | | | | |
| | None | — | — | — | 0.3% | 0.3% | 0.3% |
| | — | — | — | — | PE | PE | PE |
| | — | 0.75% | 1.50% | 2.50% | — | 1.5% | 0.2% |
| Days | — | CHDM-D90 | CHDM-D90 | CHDM-D90 | — | CHDM-D90 | octanediol |
| Pseudomonas aeruginosa | | | | | | | |
| 0 | 174000 | 174000 | 174000 | 174000 | 174000 | 174000 | 174000 |
| 7 | >100000 | 160 | 140 | 4 | >100000 | 0 | 140 |
| 14 | >100000 | 16 | 24 | 0 | 68000 | 0 | 26 |
| 30 | >100000 | 0 | 0 | 0 | 31000 | 0 | 0 |
| Staphylococcus aureus | | | | | | | |
| 0 | 184000 | 184000 | 184000 | 184000 | 184000 | 184000 | 184000 |
| 7 | >100000 | 410 | 0 | 2 | >100000 | 0 | 0 |
| 14 | >100000 | 30 | 0 | 0 | >100000 | 0 | 0 |
| 30 | >100000 | 6 | 0 | 0 | >100000 | 0 | 0 |
| Candida albicans | | | | | | | |
| 0 | 202000 | 202000 | 202000 | 202000 | 202000 | 202000 | 202000 |
| 7 | >100000 | 220 | 110 | 0 | >100000 | 170 | 4 |
| 14 | >100000 | 0 | 0 | 0 | >100000 | 2 | 0 |
| 30 | >100000 | 0 | 0 | 0 | >100000 | 0 | 0 |
| Escherichia coli | | | | | | | |
| 0 | 187000 | 187000 | 187000 | 187000 | 187000 | 187000 | 187000 |
| 7 | >100000 | 1060 | 0 | 28 | >100000 | 100 | 22 |
| 14 | >100000 | 160 | 0 | 0 | >100000 | 12 | 0 |
| 30 | >100000 | 12 | 0 | 0 | >100000 | 0 | 0 |
| Burkholderia cepacia | | | | | | | |
| 0 | 179000 | 179000 | 179000 | 179000 | 179000 | 179000 | 179000 |
| 7 | >100000 | 190 | 4 | 6 | >100000 | 30 | 50 |
| 14 | >100000 | 20 | 0 | 0 | 71000 | 0 | 0 |
| 30 | >100000 | 0 | 0 | 0 | 49000 | 0 | 0 |
| Aspergillus niger | | | | | | | |
| 0 | 174000 | 174000 | 174000 | 174000 | 174000 | 174000 | 174000 |
| 7 | >100000 | 1090 | 1440 | 1270 | >100000 | 120 | 2170 |
| 14 | >100000 | 150 | 190 | 110 | >100000 | 8 | 160 |
| 30 | >100000 | 30 | 18 | 6 | >100000 | 0 | 0 |

| | Example | | | | |
|---|---|---|---|---|---|
| | 3.8 | 3.9 | 3.10 | 3.11 | 3.12 |
| | Antimicrobial System | | | | |
| | 0.05% | 0.05% | 0.005% | 0.3% | 0.15% |
| | MP | MP | IPBC | octanediol | octanediol |
| | — | 1.5% | 1.5% | — | 0.15% |
| Days | — | CHDM-D90 | CHDM-D90 | — | CHDM-D90 |
| Pseudomonas aeruginosa | | | | | |
| 0 | 174000 | 174000 | 174000 | 174000 | 174000 |
| 7 | 130 | 2 | 0 | 60 | 10 |
| 14 | 0 | 0 | 0 | 4 | 0 |
| 30 | 0 | 0 | 0 | 0 | 0 |
| Staphylococcus aureus | | | | | |
| 0 | 184000 | 184000 | 184000 | 184000 | 184000 |
| 7 | 48000 | 14 | 8 | 80 | 2 |
| 14 | 90 | 2 | 0 | 40 | 6 |
| 30 | 50 | 0 | 0 | 0 | 0 |
| Candida albicans | | | | | |
| 0 | 202000 | 202000 | 202000 | 202000 | 202000 |
| 7 | 86000 | 0 | 70 | >100000 | 160 |
| 14 | 7200 | 0 | 6 | >100000 | 14 |
| 30 | 40 | 0 | 0 | >100000 | 0 |
| Escherichia coli | | | | | |
| 0 | 187000 | 187000 | 187000 | 187000 | 187000 |
| 7 | 6600 | 0 | 90 | 1500 | 40 |
| 14 | 510 | 0 | 6 | 90 | 6 |
| 30 | 30 | 0 | 0 | 40 | 0 |
| Burkholderia cepacia | | | | | |
| 0 | 179000 | 179000 | 179000 | NT | NT |
| 7 | 1400 | 10 | 6 | NT | NT |
| 14 | 200 | 0 | 0 | NT | NT |
| 30 | 26 | 0 | 0 | NT | NT |
| Aspergillus niger | | | | | |
| 0 | 174000 | 174000 | 174000 | 174000 | 174000 |
| 7 | >100000 | 490 | 1300 | >100000 | 4900 |
| 14 | >100000 | 120 | 140 | >100000 | 810 |
| 30 | >100000 | 14 | 12 | >100000 | 120 |

PE = phenoxyethanol
MP = methylparaben;
NT = Not tested

In these experiments, the following results were unexpected:
For the experiment with 1.5% CHDM-D90 in combination with 0.3% PE against Pseudomonas aeruginosa and Aspergillus niger, in light of the individual results for 0.3% PE and 1.5% CHDM-D90, PE at 0.3% provided very little antimicrobial activity; 1.5% CHDM alone provided significant activity; but the combination reduced the P. aeruginosa colony count to zero within seven days and the A. niger colony count to zero within 30 days.
For the experiment with 1.5% CHDM-D90 in combination with 0.05% methylparaben (MP) against fungi, Candida albicans and Aspergillus niger. MP is known to be effective against fungi, but not at this low concentration (0.05%), as can be seen from the results for 0.05% MP alone (especially against A. niger).

### Example 4

Antimicrobial efficacy data (Table 54 & Table 55) were obtained for 1,4-CHDM with and without biocide in protection of B-100 biodiesel from microbial growth derived via either biodiesel-acclimated bioslime or trivalent bacterial-fungal inocula after 15-day exposure at 22° C. This testing was via a visual turbidity methodology. Neither micro-liter plates nor automatic plate reader could be used in this experiment due to the inherent biphasic nature of the system. B-100 biodiesel:Bushnell-Haas broth was used, which is a minimal salts medium specially designed for evaluating growth of microorganisms on hydrocarbons. Samples were evaluated visually (i.e., the more the turbidity, the more the growth, the less the turbidity, the less the growth, and no turbidity means no growth). Of particular note was that 1,4-CHDM enhanced the preservative (inhibitory) action of the Killem biocide (obtained from FPPF Chemical, Buffalo, N.Y.) @200 ppm dose when the 1,4-CHDM was at 0.2-0.5 wt % concentration. No enhancement is seen at lower doses of the biocide (50 ppm or 100 ppm) nor at a lower concentration of 1,4-CHDM (0.1 wt %).

TABLE 54
15-Day Study: 1,4-CHDM-Assisted Biofouling
Control in B-100 Biodiesel @ 22°
C. (w/Agitation) (Bioslime Inoculum Set)

| | | Inoc- | Biodiesel | Biocide | | 1,4-CHDM | |
|---|---|---|---|---|---|---|---|
| Tube # | Growth¹ | ulum² | Dose³ | Dose⁴ | | Dose | |
| 1A, B, C | 0 | 0 uL | 10% (v/v) | 0 | mg/L | 0% | (v/v) |
| 2A, B, C | 3 | 50 uL | 10% (v/v) | 0 | mg/L | 0% | (v/v) |
| 3A, B, C | 3 | 50 uL | 10% (v/v) | 0 | mg/L | 0.1% | (v/v) |
| 4A, B, C | 3 | 50 uL | 10% (v/v) | 0 | mg/L | 0.2% | (v/v) |
| 5A, B, C | 3 | 50 uL | 10% (v/v) | 0 | mg/L | 0.3% | (v/v) |
| 6A, B, C | 3 | 50 uL | 10% (v/v) | 0 | mg/L | 0.4% | (v/v) |
| 7A, B, C | 3 | 50 uL | 10% (v/v) | 0 | mg/L | 0.5% | (v/v) |
| 8A, B, C | 3 | 50 uL | 10% (v/v) | 50 | mg/L | 0% | (v/v) |
| 9A, B, C | 2 | 50 uL | 10% (v/v) | 50 | mg/L | 0.1% | (v/v) |
| 10A, B, C | 2 | 50 uL | 10% (v/v) | 50 | mg/L | 0.2% | (v/v) |
| 11A, B, C | 2 | 50 uL | 10% (v/v) | 50 | mg/L | 0.3% | (v/v) |
| 12A, B, C | 2 | 50 uL | 10% (v/v) | 50 | mg/L | 0.4% | (v/v) |
| 13A, B, C | 2 | 50 uL | 10% (v/v) | 50 | mg/L | 0.5% | (v/v) |
| 14A, B, C | 2 | 50 uL | 10% (v/v) | 100 | mg/L | 0% | (v/v) |
| 15A, B, C | 2 | 50 uL | 10% (v/v) | 100 | mg/L | 0.1% | (v/v) |
| 16A, B, C | 2 | 50 uL | 10% (v/v) | 100 | mg/L | 0.2% | (v/v) |
| 17A, B, C | 2 | 50 uL | 10% (v/v) | 100 | mg/L | 0.3% | (v/v) |
| 18A, B, C | 2 | 50 uL | 10% (v/v) | 100 | mg/L | 0.4% | (v/v) |
| 19A, B, C | 2 | 50 uL | 10% (v/v) | 100 | mg/L | 0.5% | (v/v) |
| 20A, B, C | 2 | 50 uL | 10% (v/v) | 200 | mg/L | 0% | (v/v) |
| 21A, B, C | 2 | 50 uL | 10% (v/v) | 200 | mg/L | 0.1% | (v/v) |
| 22A, B, C | 1 | 50 uL | 10% (v/v) | 200 | mg/L | 0.2% | (v/v) |
| 23A, B, C | 0 | 50 uL | 10% (v/v) | 200 | mg/L | 0.3% | (v/v) |
| 24A, B, C | 0 | 50 uL | 10% (v/v) | 200 | mg/L | 0.4% | (v/v) |
| 25A, B, C | 0 | 50 uL | 10% (v/v) | 200 | mg/L | 0.5% | (v/v) |

¹GROWTH RATING
(Visual Turbidity)
0 = No Growth
1 = Slight Growth
2 = Moderate Growth
3 = Heavy Growth
²Bioslime (Biodiesel-acclimated) Inoculum
³B-100 Biodiesel in Aqueous Bushnell-Haas Medium
⁴Killem ™ Biodiesel-approved Biocide

TABLE 55
15-Day Study: 1,4-CHDM-Assisted Biofouling Control
in B-100 Biodiesel @ 22° C. (w/Agitation)
(Dual Bacteria-Yeast Inoculum Set)

| | | Inoc- | Biodiesel | Biocide | | 1,4-CHDM | |
|---|---|---|---|---|---|---|---|
| Tube # | Growth¹ | ulum² | Dose³ | Dose⁴ | | Dose | |
| 26A, B, C | 0 | 0 uL | 10% (v/v) | 0 | mg/L | 0% | (v/v) |
| 27A, B, C | 3 | 50 uL | 10% (v/v) | 0 | mg/L | 0% | (v/v) |
| 28A, B, C | 3 | 50 uL | 10% (v/v) | 0 | mg/L | 0.1% | (v/v) |
| 29A, B, C | 3 | 50 uL | 10% (v/v) | 0 | mg/L | 0.2% | (v/v) |
| 30A, B, C | 3 | 50 uL | 10% (v/v) | 0 | mg/L | 0.3% | (v/v) |
| 31A, B, C | 3 | 50 uL | 10% (v/v) | 0 | mg/L | 0.4% | (v/v) |
| 32A, B, C | 3 | 50 uL | 10% (v/v) | 0 | mg/L | 0.5% | (v/v) |
| 33A, B, C | 3 | 50 uL | 10% (v/v) | 50 | mg/L | 0% | (v/v) |
| 34A, B, C | 3 | 50 uL | 10% (v/v) | 50 | mg/L | 0.1% | (v/v) |
| 35A, B, C | 3 | 50 uL | 10% (v/v) | 50 | mg/L | 0.2% | (v/v) |
| 36A, B, C | 3 | 50 uL | 10% (v/v) | 50 | mg/L | 0.3% | (v/v) |
| 37A, B, C | 3 | 50 uL | 10% (v/v) | 50 | mg/L | 0.4% | (v/v) |
| 38A, B, C | 3 | 50 uL | 10% (v/v) | 50 | mg/L | 0.5% | (v/v) |
| 39A, B, C | 2 | 50 uL | 10% (v/v) | 100 | mg/L | 0% | (v/v) |
| 40A, B, C | 2 | 50 uL | 10% (v/v) | 100 | mg/L | 0.1% | (v/v) |
| 41A, B, C | 2 | 50 uL | 10% (v/v) | 100 | mg/L | 0.2% | (v/v) |
| 42A, B, C | 2 | 50 uL | 10% (v/v) | 100 | mg/L | 0.3% | (v/v) |
| 43A, B, C | 2 | 50 uL | 10% (v/v) | 100 | mg/L | 0.4% | (v/v) |
| 44A, B, C | 2 | 50 uL | 10% (v/v) | 100 | mg/L | 0.5% | (v/v) |
| 45A, B, C | 1 | 50 uL | 10% (v/v) | 200 | mg/L | 0% | (v/v) |
| 46A, B, C | 1 | 50 uL | 10% (v/v) | 200 | mg/L | 0.1% | (v/v) |
| 47A, B, C | 0 | 50 uL | 10% (v/v) | 200 | mg/L | 0.2% | (v/v) |
| 48A, B, C | 0 | 50 uL | 10% (v/v) | 200 | mg/L | 0.3% | (v/v) |
| 49A, B, C | 0 | 50 uL | 10% (v/v) | 200 | mg/L | 0.4% | (v/v) |
| 50A, B, C | 0 | 50 uL | 10% (v/v) | 200 | mg/L | 0.5% | (v/v) |

¹GROWTH RATING
(Visual Turbidity)
0 = No Growth
1 = Slight Growth
2 = Moderate Growth
3 = Heavy Growth
²Trivalent: 2 Bacteria & 1 Yeast (Biodiesel-acclimated) Inoculum
³B-100 Biodiesel in Aqueous Bushnell-Haas Medium
⁴Killem ™ Biodiesel-approved Biocide

### Example 5

Experiments were also conducted using Corynebacterium xerosis, which is a bacterium known to cause body odor. CHDM in combination with ethylhexyl glycerin (EHG) or in combination with triclosan (TRI) was utilized. The Corynebacterium xerosis bacterium used in this example was ATCC #373. The seed culture was grown in brain heart infusion medium. Assays were performed in brain heart infusion media in 96-well plates as described in Example 2. The brain heart infusion medium was at a pH of about 7.4. All growth was conducted at 37° C.

TABLE 56
Synergy testing of combination of Ethylhexyl
Glycerin (EHG) and CHDM with C. xerosis

| | | | Q_{A/a} | Q_{B/b} | | | | |
|---|---|---|---|---|---|---|---|---|
| Organism | Plate # | Day | (CHDM) | (EHG) | SI | B/A | % CHDM | % EHG |
| C. xerosis | EX194-195- | 2 | 2.110 | 0.000 | 1.00 | | 100.0 | 0.0 |
| | EHG-Syn1 | | | | | | | |
| C. xerosis | EX194-195- | 2 | 1.870 | 0.053 | 1.39 | 0.029 | 88.6 | 50.0 |
| | EHG-Syn1 | | | | | | | |
| C. xerosis | EX194-195- | 2 | 1.410 | 0.053 | 1.17 | 0.038 | 66.8 | 50.0 |
| | EHG-Syn1 | | | | | | | |
| C. xerosis | EX194-195- | 2 | 1.870 | 0.040 | 1.26 | 0.021 | 88.6 | 37.5 |
| | EHG-Syn1 | | | | | | | |
| C. xerosis | EX194-195- | 2 | 0.000 | 0.107 | 1.00 | | 0.0 | 100.0 |
| | EHG-Syn1 | | | | | | | |
| C. xerosis | EX194-195- | 3 | 2.110 | 0.000 | 1.00 | | 100.0 | 0.0 |
| | EHG-Syn1 | | | | | | | |
| C. xerosis | EX194-195- | 3 | 1.410 | 0.040 | 1.17 | 0.028 | 66.8 | 50.0 |
| | EHG-Syn1 | | | | | | | |
| C. xerosis | EX194-195- | 3 | 1.410 | 0.053 | 1.33 | 0.038 | 66.8 | 66.3 |
| | EHG-Syn1 | | | | | | | |
| C. xerosis | EX194-195- | 3 | 1.410 | 0.030 | 1.04 | 0.021 | 66.8 | 37.5 |
| | EHG-Syn1 | | | | | | | |
| C. xerosis | EX194-195- | 3 | 0.000 | 0.080 | 1.00 | | 0.0 | 100.0 |
| | EHG-Syn1 | | | | | | | |

TABLE 57
Synergy testing of combination of Triclosan (TRI) and CHDM with C. xerosis

| | | | Q_{A/a} | Q_{B/b} | | | | |
|---|---|---|---|---|---|---|---|---|
| Organism | Plate # | Day | (CHDM) | (TRI) | SI | B/A | % CHDM | % TRI |
| C. xerosis | EX194-195- | 2 | 2.110 | 0.00 | 1.00 | | 100.0 | 0.0 |
| | TRI-Syn1 | | | | | | | |
| C. xerosis | EX194-195- | 2 | 1.870 | 0.0087 | 1.39 | 0.0047 | 88.8 | 50.3 |
| | TRI-Syn1 | | | | | | | |
| C. xerosis | EX194-195- | 2 | >1.410 | >0.0087 | >1.17 | — | >66.8 | >50.3 |
| | TRI-Syn1 | | | | | | | |
| C. xerosis | EX194-195- | 2 | 1.870 | 0.0065 | 1.26 | 0.0035 | 88.6 | 37.6 |
| | TRI-Syn1 | | | | | | | |
| C. xerosis | EX194-195- | 2 | 0.00 | 0.0173 | 1.00 | | 0.0 | 100.0 |
| | TRI-Syn1 | | | | | | | |
| C. xerosis | EX194-195- | 3 | 2.110 | 0.00 | 1.00 | | 100.0 | 0.0 |
| | TRI-Syn1 | | | | | | | |
| C. xerosis | EX194-195- | 3 | 1.870 | 0.0087 | 1.39 | 0.0047 | 88.8 | 50.3 |
| | TRI-Syn1 | | | | | | | |
| C. xerosis | EX194-195- | 3 | >1.410 | >0.0087 | >1.17 | — | >66.8 | >50.3 |
| | TRI-Syn1 | | | | | | | |
| C. xerosis | EX194-195- | 3 | 1.870 | 0.0065 | 1.26 | 0.0035 | 88.6 | 37.6 |
| | TRI-Syn1 | | | | | | | |
| C. xerosis | EX194-195- | 3 | 0.00 | 0.0173 | 1.00 | | 0.0 | 100.0 |
| | TRI-Syn1 | | | | | | | |

Synergistic effects were not observed from this data, however, the large amount of data shown in Examples 1-4 clearly show that a synergistic effect does exist when CHDM is used with other antimicrobial agents. It is not clear the reason for the lack of synergy seen in this example, but it may be due to the experimental variability of biological systems.

### Example 6

### Antimicrobial Activity Comparison of 1,1 and 1,4-Cyclohexanedimethanol

The antimicrobial activities of 1,4-cyclohexanedimethanol (1,4-CHDM) and 1,1-cyclohexanedimethanol (1,1-CHDM) have been determined. Each activity was calculated in terms of a minimum inhibitory concentration (MIC), revealing the lowest concentration necessary to inhibit visible growth. MICs were individually calculated for three consecutive days with both 1,1-cyclohexanedimethanol and the 31% cis:69% trans mixture of 1,4-cyclohexanedimethanol. Both compounds were evaluated against a panel of five strains of microorganisms. 1,1-CHDM afforded significant improvement in efficacy over 1,4-CHDM with correlation between different organisms.

Higher antimicrobial activity can allow for reduced concentrations and volumes of CHDM during formulation. Reducing the amount of CHDM can minimize the impact on the properties of the product being formulated or the finished article while retaining comparable activity and can also reduce costs by producing less material with the same net activity.

### Materials and Methods for Example 6

Strains P. aeruginosa, C. albicans, E. coli, A. niger and S. aureus were purchased from the American Type Culture Collection (Manassas, Va.). NUNC flat bottom polystyrene 96 well microtiter plates (NUNC Cat# 269787), and 17×100 mm culture tubes (VWR Cat# 60818-703) were purchased from VWR International, LLC (West Chester, Pa.). Eastman CHDM-D90 and 1,1-CHDM (>99.7% by GC and verified by NMR) were provided by Eastman Chemical Company (Kingsport, Tenn.). All bacterial cultures were grown in BD BBL trypticase soy broth, and all fungal cultures were grown in sabourand dextrose broth purchased from VWR International, LLC (West Chester, Pa.). Absorbance measurements were taken with a TECAN GENios Pro microplate reader.

### Preparation of Inoculum

A small loopful of inoculum was transferred from a freshly streaked agar plate of each strain to 5 ml of sterile media in a 17×100 mm culture tube. The tubes were incubated without shaking at the appropriate temperature and in the appropriate medium as listed in Table 58. The bacteria were incubated for 20-28 hours and C. albicans for 44-52 hours.

The procedure for A. niger was significantly different. A. niger was cultured on sabourand dextrose agar plates until a heavy concentration of black spores were visibly apparent. Spores were harvested from the plate by suspension in 3 ml of sabourand dextrose broth utilizing a sterile plastic spreader and sterile transfer pipette.

TABLE 58
Microorganisms utilized for MIC determination

| | | Incubation | | |
|---|---|---|---|---|
| Genus and | ATCC | temperature | | Growth |
| species | ID | (° C.) | Description | Medium |
| Pseudomonas | 27853 | 30 | Gram (−) rod- | Trypticase soy |
| aeruginosa | | | shaped | broth |
| | | | bacterium | |
| Candida | 10231 | 25 | Diploid fungus | Sabourand |
| albicans | | | | dextrose broth |
| Escherichia | 25922 | 35 | Gram (−) rod- | Trypticase soy |
| coli | | | shaped | broth |
| | | | bacterium | |
| Aspergillus | 16404 | 25 | Filamentous | Sabourand |
| niger | | | fungus | dextrose broth |
| Staphylococcus | 25923 | 35 | Gram (+) | Trypticase soy |
| aureus | | | spherical- | broth |
| | | | shaped | |
| | | | bacterium | |

### Dilution of CHDM Isomers

Stock solutions were prepared for each isomer in the corresponding growth media at a concentration of 5% w/v (1,4-CHDM) or 2.25% w/v (1,1-CHDM). Serial dilutions were prepared with a dilution ratio of 1:1.3333 such that one log range was covered with nine dilutions.

### Preparation of 96 Well Plates

Two-hundred microliters of each CHDM concentration was transferred into 4 wells of a sterile 96-well plate. Four extra wells of the highest concentration were filled for the uninoculated high-level controls. Eight additional wells were filled with only sterile broth to serve as negative and positive controls. Three of the four wells for each CHDM concentration were inoculated with one of the test strains listed in Table 58. The last well of each CHDM isomer dilution was left uninoculated to serve as controls for background turbidity associated with test compounds. Plates with bacteria or C. albicans were inoculated with 2 μl of seed culture for final concentration of roughly 10⁶ CFU/ml for the bacteria and 10⁵ CFU/ml for the C. albicans. Plates with A. niger were inoculated with 2 μl of spore suspension prepared above.

### Determination of Minimum Inhibitory Concentration (MIC)

Each plate was covered and incubated at the appropriate temperature and turbidity as a measure of cell density was determined via absorbance measurement at 612 nm using a microplate reader. Measurements were taken at 24, 48 and 72 hours for each plate. The raw data was exported into an Excel spreadsheet and the MIC values were determined and expressed as wt %. The absorbance of each inoculated CHDM well was retrieved by first subtracting out the average reading for each uninoculated well, then by comparison to a positive threshold to determine positive or negative status for growth. The positive threshold was calculated by multiplication of the average absorbance for the inoculated media-only wells by 0.05. The MIC was determined as the lowest test concentration resulting in all three replicate wells displaying values below the positive threshold.

### Results

1,1-cyclohexanedimethanol exhibited a measurable increase in antimicrobial efficacy over that of 1,4-cyclohexanedimethanol. Antimicrobial efficacy increased against four of the five test organisms in these experiments. The solubility of 1,1-CHDM was limited to 2.25% (w/v) in aqueous growth media, therefore comprehensive MIC results were limited to the range of 0-2.25%. Final results have been summarized below in Table 59.

TABLE 59
MIC data - Compare 1,1 and 1,4 CHDM at 24, 48 and 72 H

| Organism | Isomer | MIC Day 1 | MIC Day 2 | MIC Day 3 |
|---|---|---|---|---|
| P. aeruginosa | 1,4 CHDM | 1.58 | 1.58 | 2.11 |
| | 1,1 CHDM | 1.26 | 1.26 | 1.26 |
| C. albicans | 1,4 CHDM | 3.75 | 4.99 | >5.0 |
| | 1,1 CHDM | 2.25 | 2.25 | 2.25 |
| E. coli | 1,4 CHDM | 1.58 | 1.58 | 1.58 |
| | 1,1 CHDM | 1.26 | 1.26 | 1.26 |
| A. niger | 1,4 CHDM | >5.0 | 3.75 | 3.75 |
| | 1,1 CHDM | >2.25 | >2.25 | >2.25 |
| S. aureus | 1,4 CHDM | 3.75 | 3.75 | 3.75 |
| | 1,1 CHDM | 2.25 | 2.25 | 2.25 |

These results show that 1,1-CHDM can be a more effective antimicrobial agent than its structural isomer 1,4-CHDM as shown by the lower MIC values. The invention has been described in detail with particular reference to preferred embodiments thereof, but it will be understood that variations and modifications can be effected within the spirit and scope of the invention.

That which is claimed is:

1. A method for enhancing the effectiveness of at least one antimicrobial agent in reducing or inhibiting microbial growth in an aqueous composition comprising:
adding at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and said antimicrobial agent to said aqueous composition.

2. The method according to claim 1, wherein said cycloaliphatic diol antimicrobial agent is added in an amount of about 0.2 to about 5 weight percent, based on the total weight of said aqueous composition.

3. The method according to claim 1, wherein said cycloaliphatic diol antimicrobial agent is added to said aqueous composition by contacting said aqueous composition with a solvent that is immiscible with water and that comprises said antimicrobial agent.

4. The method according to claim 1, wherein said aqueous composition comprises an organic compound selected from hydrocarbons, triglycerides, fatty acids, fatty acid alkyl esters, fatty alcohols, polyglycol ethers, alkyl glycol ethers, alkyl glycol esters, alkyl glycol ether esters, alkyl amines, alkyl amides, or mixtures thereof.

5. The method according to claim 4, wherein said organic compound is diesel, biodiesel, a mixture of diesel and biodiesel, aviation fuel, hydraulic oil, lubrication oil, vegetable oil, crude oil, transmission fluid, heating oil, or kerosene.

6. A composition comprising:
(a) at least one fuel or oil selected from diesel, biodiesel, a mixture of diesel and biodiesel, aviation fuel, hydraulic oil, lubrication oil, vegetable oil, crude oil, transmission fluid, heating oil, or kerosene;
(b) at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and
(c) at least one other antimicrobial agent.

7. A personal care product comprising at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol and at least one other antimicrobial agent.

8. The personal care product according to claim 7 wherein said cycloaliphatic diol antimicrobial agent is added in an amount ranging from about 1 to about 5 percent by weight.

9. A medicated product comprising:
at least one medicinal substance;
at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and
at least one other antimicrobial agent.

10. The medicated product according to claim 9 wherein said cycloaliphatic diol antimicrobial agent is present in an amount ranging from about 1% to about 5% by weight.

11. An animal care product comprising:
at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and
at least one other antimicrobial agent.

12. The animal care product according to claim 11 wherein the amount of said cycloaliphatic diol antimicrobial agent ranges from about 1 to about 5 percent by weight.

12. A household care product comprising:
at least one cycloaliphatic diol antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and
at least one other antimicrobial agent.

13. The household care product according to claim 12, which comprises about 1 to about 5 weight percent of said cycloaliphatic diol antimicrobial agent.

14. A method for providing residual antimicrobial activity to a surface, said method comprising:
topically applying said product according to claim 7, 9, 11, or 12 to the surface; and
optionally removing any excess amounts of said product from the surface.

15. A method for preventing or treating a bacterial or fungal infection on a mammalian surface, said method comprising:
topically applying said product according to claim 7, 9, or 11 to said mammalian surface; and
optionally removing any excess amounts of said product from said mammalian surface.

16. A method for preventing or reducing odor from the presence of bacteria or fungi on a mammalian surface, said method comprising:
topically applying said product according to claim 7, 9, or 11 to said mammalian surface; and
optionally removing any excess amounts of said product from said mammalian surface.

17. A method for providing antimicrobial activity to a film, fiber, molded or extruded article, or composite material made of fibers, polymers, adhesives, and/or gypsum; said method comprising:
incorporating an antimicrobial agent selected from the group consisting of 1,1-cyclohexanedimethanol, 1,2-cyclohexanedimethanol, 1,4-cyclohexanedimethanol, and 2,2,4,4-tetramethyl-1,3-cyclobutanediol; and
at least one other antimicrobial agent into said film, fiber, molded or extruded article, or composite material during its manufacturing process.

18. The method according to claim 17, which prevents a biofilm from forming on a surface of the film, fiber, molded or extruded article, or composite material.

19. The method according to claim 17, wherein said antimicrobial agent is incorporated in an amount of about 1 to about 5 weight percent, based on the total weight of the film, fiber, molded or extruded article, or composite material.
